SEQUENCE LISTING (17 sequences)

SEQ ID NO: 1 - protein, 90 aa, Artificial Sequence
  “N” region of apoferritin
MSSQIRQNYSTDVEAAVNSLVNLYLQASYTYLSLGFYFDRDDVALEGVSHFFRELAEEKREGYERLLKMQNQRGGRALFQDIKKPAEDEW

SEQ ID NO: 2 - protein, 85 aa, Artificial Sequence
  “C” region of apoferritin
GKTPDAMKAAMALEKKLNQALLDLHALGSARTDPHLCDFLETHFLDEEVKLIKKMGDHLTNLHRLGGPEAGLGEYLFERLTLRHD

SEQ ID NO: 3 - protein, 70 aa, Artificial Sequence
  Linker
GGGGSGGGGSGGGGSGGGGSGGGGSGGGGSGGGGSGGGGSGGGGSGGGGSGGGGSGGGGSGGGGSGGGGS

SEQ ID NO: 4 - protein, 27 aa, Artificial Sequence
  Linker
GGGGSGGGGSGGGGSGGGGSGGGGSGG

SEQ ID NO: 5 - protein, 26 aa, Artificial Sequence
  Linker
GGSGGSGGSGGSGGGSGGSGGSGGSG

SEQ ID NO: 6 - protein, 175 aa, Homo sapiens
MSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 7 - protein, 621 aa, Artificial Sequence
  N49P7_N-Ferritin
QSALTQPRSVSASPGQSVTISCTGTHNLVSWCQHQPGRAPKLLIYDFNKRPSGVPDRFSGSGSGGTASLTITGLQDDDDAEYFCWAYEAFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECGGGGSGGGGSGGGGSGGGGSGGGGSGGGGSGGGGSGGGGSGGGGSGGGGSGGGGSGGGGSGGGGSGGGGSADLVQSGAVVKKPGDSVRISCEAQGYRFPDYIIHWIRRAPGQGPEWMGWMNPMGGQVNIPWKFQGRVSMTRDTSIETAFLDLRGLKSDDTAVYYCVRDRSNGSGKRFESSNWFLDLWGRGTAVTIQSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSGGGGSGGGGSGGMSSQIRQNYSTDVEAAVNSLVNLYLQASYTYLSLGFYFDRDDVALEGVSHFFRELAEEKREGYERLLKMQNQRGGRALFQDIKKPAEDEW

SEQ ID NO: 8 - protein, 736 aa, Artificial Sequence
  PGDM1400_hFerritin
DFVLTQSPHSLSVTPGESASISCKSSHSLIHGDRNNYLAWYVQKPGRSPQLLIYLASSRASGVPDRFSGSGSDKDFTLKISRVETEDVGTYYCMQGRESPWTFGQGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSGGGGSGGGGSGGGGSGGGGSGGGGSGGGGSQAQLVQSGPEVRKPGTSVKVSCKAPGNTLKTYDLHWVRSVPGQGLQWMGWISHEGDKKVIVERFKAKVTIDWDRSTNTAYLQLSGLTSGDTAVYYCAKGSKHRLRDYALYDDDGALNWAVDVDYLSNLEFWGQGTAVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSGGGGSGGGGSGGGGSGGMSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 9 - protein, 881 aa, Artificial Sequence
  10E8v4_C-Ferritin_Fc1
SELTQDPAVSVALKQTVTITCRGDSLRSHYASWYQKKPGQAPVLLFYGKNNRPSGIPDRFSGSASGNRASLTITGAQAEDEADYYCSSRDKSGSRLSVFGGGTKLTVLSQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECGGGGSGGGGSGGGGSGGGGSGGGGSGGGGSGGGGSGGGGSGGGGSGGGGSGGGGSGGGGSGGGGSGGGGSEVRLVESGGGLVKPGGSLRLSCSASGFDFDNAWMTWVRQPPGKGLEWVGRITGPGEGWSVDYAESVKGRFTISRDNTKNTLYLEMNNVRTEDTGYYFCARTGKYYDFWSGYPPGEEYFQDWGQGTLVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSGGGGSGGGGSGGKTPDAMKAAMALEKKLNQALLDLHALGSARTDPHLCDFLETHFLDEEVKLIKKMGDHLTNLHRLGGPEAGLGEYLFERLTLRHDGGGGSGGGGSGGGGSGGGGSGGGGSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 883 aa, Artificial Sequence
  10E8v4_C-Ferritin_Fc1-LS
SELTQDPAVSVALKQTVTITCRGDSLRSHYASWYQKKPGQAPVLLFYGKNNRPSGIPDRFSGSASGNRASLTITGAQAEDEADYYCSSRDKSGSRLSVFGGGTKLTVLSQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECGGGGSGGGGSGGGGSGGGGSGGGGSGGGGSGGGGSGGGGSGGGGSGGGGSGGGGSGGGGSGGGGSGGGGSEVRLVESGGGLVKPGGSLRLSCSASGFDFDNAWMTWVRQPPGKGLEWVGRITGPGEGWSVDYAESVKGRFTISRDNTKNTLYLEMNNVRTEDTGYYFCARTGKYYDFWSGYPPGEEYFQDWGQGTLVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSGGGGSGGGGSGGGGKTPDAMKAAMALEKKLNQALLDLHALGSARTDPHLCDFLETHFLDEEVKLIKKMGDHLTNLHRLGGPEAGLGEYLFERLTLRHDGGSGGSGGSGGSGGGSGGSGGSGGSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 11 - protein, 4 aa, Artificial Sequence
  Linker
GGGS

SEQ ID NO: 12 - protein, 5 aa, Artificial Sequence
  Linker
GGGGS

SEQ ID NO: 13 - protein, 1178 aa, Artificial Sequence
  10E8v4_C-Ferritin_Fc
SELTQDPAVSVALKQTVTITCRGDSLRSHYASWYQKKPGQAPVLLFYGKNNRPSGIPDRFSGSASGNRASLTITGAQAEDEADYYCSSRDKSGSRLSVFGGGTKLTVLSQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECGGGGSGGGGSGGGGSGGGGSGGGGSGGGGSGGGGSGGGGSGGGGSGGGGSGGGGSGGGGSGGGGSGGGGSEVRLVESGGGLVKPGGSLRLSCSASGFDFDNAWMTWVRQPPGKGLEWVGRITGPGEGWSVDYAESVKGRFTISRDNTKNTLYLEMNNVRTEDTGYYFCARTGKYYDFWSGYPPGEEYFQDWGQGTLVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSGGGGSGGGGSGGKTPDAMKAAMALEKKLNQALLDLHALGSARTDPHLCDFLETHFLDEEVKLIKKMGDHLTNLHRLGGPEAGLGEYLFERLTLRHDGGGGSGGGGSGGGGSGGGGSGGGGSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSGGGGSGGGGSGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 8 aa, Artificial Sequence
  Flag tag
DYKDDDDK

SEQ ID NO: 15 - protein, 89 aa, Artificial Sequence
  “N” region of apoferritin
SSQIRQNYSTDVEAAVNSLVNLYLQASYTYLSLGFYFDRDDVALEGVSHFFRELAEEKREGYERLLKMQNQRGGRALFQDIKKPAEDEW

SEQ ID NO: 16 - protein, 85 aa, Artificial Sequence
  “C” region of apoferritin
GKTPDAMKAAMALEKKLNQALLDLHALGSARTDPHLCDFLETHFLDEEVKLIKKMGDHLTNLHRLGGPEAGLGEYLFERLTLKHD

SEQ ID NO: 17 - protein, 734 aa, Artificial Sequence
  PGDM1400_hFerritin
DFVLTQSPHSLSVTPGESASISCKSSHSLIHGDRNNYLAWYVQKPGRSPQLLIYLASSRASGVPDRFSGSGSDKDFTLKISRVETEDVGTYYCMQGRESPWTFGQGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSGGGGSGGGGSGGGGSGGGGSGGGGSGGGGSQAQLVQSGPEVRKPGTSVKVSCKAPGNTLKTYDLHWVRSVPGQGLQWMGWISHEGDKKVIVERFKAKVTIDWDRSTNTAYLQLSGLTSGDTAVYYCAKGSKHRLRDYALYDDDGALNWAVDVDYLSNLEFWGQGTAVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSGGGGSGGGGSGGSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLKHD

(19) United States
(12) Patent Application Publication
(10) Pub. No.: US 2024/0317842 A1
(43) Pub. Date: Sep. 26, 2024

(54) MULTABODY CONSTRUCTS, COMPOSITIONS, AND METHODS

(71) Applicant: THE HOSPITAL FOR SICK CHILDREN, Toronto (CA)

(72) Inventors: Jean-Philippe Julien, Toronto (CA); Edurne Rujas Diez, Vitoria (ES)

(21) Appl. No.: 18/263,217

(22) PCT Filed: Jan. 28, 2022
(86) PCT No.: PCT/CA2022/050122

§ 371 Date: Jul. 27, 2023

Related U.S. Application Data
(60) Provisional application No. 63/142,704, filed on Jan. 28, 2021.

(51) Int. Cl. C07K 16/10; A61K 39/00; A61P 31/18; C07K 14/47

(52) U.S. Cl. CPC C07K 16/1063; A61P 31/18; C07K 14/47; A61K 2039/505; C07K 2317/55; C07K 2317/76; C07K 2317/94; C07K 2319/30

(57) ABSTRACT

In aspects, a fusion protein comprises a nanocage monomer or a subunit thereof linked to an Fc monomer, wherein a plurality of the fusion proteins are capable of self-assembling to form a nanocage comprising one or more Fc dimers. In aspects, a fusion protein comprises a nanocage monomer or a subunit thereof linked to an Fc monomer or scFc fragment at the C-terminus of the nanocage monomer or subunit thereof, wherein a plurality of the fusion proteins are capable of self-assembling to form a nanocage.

### CROSS-REFERENCE TO RELATED APPLICATIONS

The present application claims the benefit of and priority to U.S. Provisional Patent Application No. 63/142,704, filed Jan. 28, 2021, the entire content of which is hereby incorporated by reference in its entirety for all purposes.

### SEQUENCE LISTING

The instant application contains a Sequence Listing which has been submitted electronically in ASCII format and is hereby incorporated by reference in its entirety. Said ASCII copy, created on Jan. 28, 2022, is named “Sequence Listing Jan-2022 3206-5048_ST25.txt” and is 8100 bytes in size.

### FIELD

The present invention relates to polypeptides. In particular, the present invention relates to Multabody polypeptides and related constructs, compositions, and methods.

### BACKGROUND

Nanoparticles have contributed to advancements in various disciplines. Their use has the potential to confer targeted delivery and allows the engineering of ordered micro-arrays, slow release and caged micro-environments for catalytic processes.

For the fabrication of nanoparticles that contain sensitive and metastable proteins, protein self-assembly is an attractive method. Indeed, self-assembled nanoparticles form under physiological conditions through non-covalent interactions and reliably yield uniform and often symmetric nanocapsules or nanocages. Self-assembling protein nanoparticles possess three distinct surfaces that can all be tweaked to convey added functionalities: exterior, interior and inter-subunits surfaces.

Fusion proteins comprising self-assembling proteins have been described. For example, it is known to display antigens on the exterior surface of assembled nanocages for use as vaccines.

A need exists for improved constructs, compositions, and methods for designing nanoparticles and treating and/or preventing disease.

### SUMMARY OF THE INVENTION

In accordance with an aspect, there is provided a fusion protein comprising a first nanocage monomer or a subunit thereof linked to an Fc monomer, wherein a plurality of the fusion proteins are capable of self-assembling to form a nanocage comprising one or more Fc dimers.

In an aspect, the Fc monomer is linked to the first nanocage monomer or subunit thereof at the N- or C-terminus of the first nanocage monomer or subunit thereof, preferably at the C-terminus.

In an aspect, the subunit comprises an N-subunit or a C-subunit, corresponding substantially to the N-terminal half of a nanocage monomer and the C-terminal half of a nanocage monomer, respectively, wherein the N-subunit and the C-subunit are capable of self-assembling to form a nanocage monomer.

In an aspect, the Fc monomer is linked to the N-subunit or C-subunit at the C-terminus of the N-subunit or C-subunit, preferably wherein the Fc monomer is linked to the C-subunit at the C-terminus.

In an aspect, the first nanocage monomer or subunit thereof is further linked to a first bioactive moiety.

In an aspect, the first bioactive moiety is linked to the first nanocage monomer or subunit thereof at the N- or C-terminus of the first nanocage monomer or subunit thereof, preferably the N-terminus.

In an aspect, the first bioactive moiety is linked to the N-subunit or C-subunit at the N-terminus of the N-subunit or C-subunit, preferably wherein the first bioactive moiety is linked to the C-subunit at the N-terminus.

In an aspect, the first bioactive moiety decorates the interior and/or exterior surface, preferably the exterior surface, of the assembled nanocage.

In an aspect, the first bioactive moiety comprises a first antigen-binding moiety.

In an aspect, the first antigen-binding moiety comprises an antibody or fragment thereof.

In an aspect, the first antigen-binding moiety comprises a Fab fragment.

In an aspect, the antibody or fragment thereof comprises a scFab fragment, a scFv fragment, a sdAb fragment, a VHH domain or a combination thereof.

In an aspect, the antibody or fragment thereof comprises a heavy and/or light chain of a Fab fragment.

In an aspect, the first antigen-binding moiety binds specifically to an antigen associated with an antibody-preventable and/or antibody-treatable condition.

In an aspect, the antigen is associated with an infectious agent, including a virus, bacteria, a parasite, a fungus, or a yeast, a cancer, or an immune disease, including an autoimmune disease.

In an aspect, the first antigen-binding moiety comprises an HIV-1 specific antigen-binding moiety.

In an aspect, the HIV-1-specific antigen-binding moiety binds to BG505 SOSIP_D368R, 93TH057 gp120, and/or an MPER peptide.

In an aspect, the HIV-1 specific antigen-binding moiety comprises an HIV-1 specific antigen-binding moiety from PGDM1400, 10E8v4, and/or N49P7.

In an aspect, the fusion protein comprises the first nanocage monomer or subunit thereof linked to the Fc monomer at the C-terminus of the first nanocage monomer or subunit thereof and linked to the first bioactive moiety at the N-terminus of the first nanocage monomer or subunit thereof.

In an aspect, the fusion protein comprises the C-subunit linked to the Fc monomer at the C-terminus of the C-subunit and linked to the first bioactive moiety at the N-terminus of the C-subunit.

In an aspect, the fusion protein is provided in combination with an N-subunit or with a fusion protein comprising an N-subunit.

In an aspect, the N-subunit is linked to a second bioactive moiety at the N- or C-terminus, preferably the N-terminus.

In an aspect, the second bioactive moiety comprises a second antigen-binding moiety, and wherein, if the first bioactive moiety comprises the first antigen-binding moiety, the second antigen-binding moiety may be the same or different from the first antigen-binding moiety.

In an aspect, the second antigen-binding moiety comprises an antibody or fragment thereof.

In an aspect, the second antigen-binding moiety comprises a Fab fragment.

In an aspect, the antibody or fragment thereof comprises a scFab fragment, a scFv fragment, a sdAb fragment, a VHH domain or a combination thereof.

In an aspect, the antibody or fragment thereof comprises a heavy and/or light chain of a Fab fragment.

In an aspect, the N-subunit is further linked to an Fc monomer at the N- or C-terminus of the N-subunit, preferably the C-terminus.

In an aspect, the Fc monomer is derived from an IgG, IgA, IgD, IgM, or IgE, and is preferably human.

In an aspect, the Fc monomer is derived from an IgG, such as IgG1, IgG2, IgG3, or IgG4.

In an aspect, the Fc monomer is an IgG1 Fc monomer.

In an aspect, the Fc monomer comprises one or more mutations or sets of mutations that modulate the half-life of the fusion protein from, for example, minutes or hours to several days, weeks, or months.

In an aspect, the Fc monomer comprises a mutation at one or more of L234, L235, G236, G237, M252, I253, S254, T256, P329, A330, M428, N434, or a combination thereof (wherein numbering is according to the EU index), such as M428L and N434S (“LS”); M252Y, S254T and T256E (“YTE”); L234A and L235A (“LALA”); I253A; L234A, L235A, and P329G (“LALAP”); G236R; G237A; and/or A330L or a combination thereof.

In an aspect, from about 3 to about 100 nanocage monomers, such as 24, 32, 48, or 60 nanocage monomers, or from about 4 to about 200 nanocage monomer subunits, such as 4, 6, 8, 10, 12, 14, 18, 20, 22, 24, 26, 28, 30, 32, 34, 36, 38, 40, 42, 44, 46, 48, 50, or more, optionally in combination with one or more whole nanocage monomers, are capable of self-assembling to form a nanocage.

In an aspect, the first nanocage monomer or subunit thereof is selected from ferritin, apoferritin, encapsulin, SOR, lumazine synthase, pyruvate dehydrogenase, carboxysome, vault proteins, GroEL, heat shock protein, E2P, MS2 coat protein, fragments thereof, and variants thereof.

In an aspect, the first nanocage monomer or subunit thereof is apoferritin, optionally human apoferritin.

In an aspect, the first nanocage monomer or subunit thereof is an apoferritin light chain, optionally human apoferritin light chain.

In an aspect, the fusion protein comprises a first apoferritin subunit, optionally a first human apoferritin subunit, and wherein the first apoferritin subunit is capable of self-assembling with a second apoferritin subunit.

In an aspect, the first and second apoferritin monomer subunits interchangeably comprise the “N” and “C” regions of apoferritin.

In an aspect, the “N” region of apoferritin comprises or consists of a sequence at least 70% (such as at least 75%, 80%, 85%, 90%, 95%, 96%, 97%, 98%, 99%, or 100%) identical to:

(SEQ ID NO: 1)
MSSQIRQNYSTDVEAAVNSLVNLYLQASYTYLSLGFYFDRDDVALEGVS
HFFRELAEEKREGYERLLKMQNQRGGRALFQDIKKPAEDEW
or
(SEQ ID NO: 15)
SSQIRQNYSTDVEAAVNSLVNLYLQASYTYLSLGFYFDRDDVALEGVSH
FFRELAEEKREGYERLLKMQNQRGGRALFQDIKKPAEDEW.

In an aspect, the “C” region of apoferritin comprises or consists of a sequence at least 70% (such as at least 75%, 80%, 85%, 90%, 95%, 96%, 97%, 98%, 99%, or 100%) identical to:

(SEQ ID NO: 2)
GKTPDAMKAAMALEKKLNQALLDLHALGSARTDPHLCDFLETHFLDEEV
KLIKKMGDHLTNLHRLGGPEAGLGEYLFERLTLRHD
or
(SEQ ID NO: 16)
GKTPDAMKAAMALEKKLNQALLDLHALGSARTDPHLCDFLETHFLDEEV
KLIKKMGDHLTNLHRLGGPEAGLGEYLFERLTLKHD.

In an aspect, the Fc monomer is linked to the first nanocage monomer or subunit thereof through a linker.

In an aspect, the first bioactive moiety is linked to the first nanocage monomer or subunit thereof through a linker.

In an aspect, the second bioactive moiety is linked to the N-subunit through a linker.

In an aspect, the linker is flexible or rigid and comprises from about 1 to about 100 amino acid residues, such as from about 1 to about 70 amino acid residues, such as from about 1 to about 30 amino acid residues, such as from about 8 to about 16 amino acid residues.

In an aspect, the linker comprises a GS domain.

In an aspect, the GS domain comprises a GS repeat, a GGS repeat, a GGGS (SEQ ID NO:11) repeat, and/or a GGGGS (SEQ ID NO:12) repeat, such as 1, 2, 3, 4, or more GGGGS (SEQ ID NO:12) repeats.

In an aspect, the linker comprises or consists of a sequence at least 70% (such as at least 75%, 80%, 85%, 90%, 95%, 96%, 97%, 98%, 99%, or 100%) identical to: GGGGSGGGGSGGGGSGGGGSGGGGSGG (SEQ ID NO:4).

(SEQ ID NO: 4)
GGGGSGGGGSGGGGSGGGGSGGGGSGG.

In an aspect, the linker comprises or consists of a sequence at least 70% (such as at least 75%, 80%, 85%, 90%, 95%, 96%, 97%, 98%, 99%, or 100%) identical to:

(SEQ ID NO: 5)
GGSGGSGGSGGSGGGSGGSGGSGGSG.

In accordance with an aspect, there is provided a nanocage comprising at least one fusion protein described herein and at least one second nanocage monomer or subunit thereof that self-assembles with the fusion protein.

In an aspect, the fusion protein comprises a first nanocage monomer subunit, the second nanocage monomer or subunit thereof is a second nanocage monomer subunit, and the second nanocage monomer subunit self-assembles with the fusion protein to form the nanocage monomer.

In an aspect, from about 1% to about 100%, such as from about 1%, 4%, 8%, 10%, 12%, 15%, 20%, 25%, 30%, 35%, 40%, 45%, 50%, 55%, 60%, 65%, 70%, 75%, 80%, 85%, 90%, or 95%, to about 4%, 8%, 10%, 12%, 15%, 20%, 25%, 30%, 35%, 40%, 45%, 50%, 55%, 60%, 65%, 70%, 75%, 80%, 85%, 90%, 95%, or 100%, such as from about 20% to about 80%, of the nanocage monomers or subunits thereof are comprised within a fusion protein described herein.

In an aspect, each nanocage monomer or subunit thereof is comprised within a fusion protein described herein.

In an aspect, the nanocage comprises 1 bioactive moiety or at least 2, 3, 4, 5, 6, 7, 8, 9, or 10 different bioactive moieties, such as 2 or 3 different bioactive moieties.

In an aspect, the nanocage is multivalent.

In an aspect, the nanocage is multispecific.

In an aspect, at least one bioactive moiety decorates the exterior surface of the nanocage and at least one Fc dimer decorates the exterior surface of the nanocage.

In an aspect, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 bioactive moieties decorate the exterior surface of the nanocage and at least two Fc dimers decorate the exterior surface of the nanocage.

In an aspect, the nanocage comprises:
(a) at least one nanocage monomer fused to a first antigen-binding moiety, such as a Fab,
(b) at least one N-subunit fused to a second antigen-binding moiety, such as a Fab, and
(c) and at least one C-subunit fused to:
   (i) a third antigen-binding moiety, such as a Fab at one terminus, and
   (ii) a Fc monomer at the other terminus.

In an aspect, each antigen-binding moiety is linked to the N-terminus of the nanocage monomer or subunit thereof and wherein the Fc monomer is linked to the C-terminus of the C-subunit.

In an aspect, the antigen-binding moiety binds specifically to an antigen associated with an antibody-preventable and/or antibody-treatable condition.

In an aspect, the antigen is associated with an infectious agent, including a virus, bacteria, a parasite, a fungus, or a yeast, a cancer, or an immune disease, including an autoimmune disease.

In an aspect, each antigen-binding moiety is a different HIV-1-specific Fab.

In an aspect, the HIV-1-specific Fab binds to BG505 SOSIP_D368R, 93TH057 gp120, and/or an MPER peptide.

In an aspect, the HIV-1 specific Fab comprises PGDM1400 Fab, 10E8v4 Fab, and/or N49P7 Fab.

In an aspect, the HIV-1 specific Fab comprises PGDM1400 Fab, 10E8v4 Fab, and N49P7 Fab.

In an aspect, the nanocage comprises at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 antigen-binding moieties.

In an aspect, the nanocage comprises at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 Fc monomers.

In an aspect, the nanocage is carrying a cargo molecule, such as a pharmaceutical agent, a diagnostic agent, and/or an imaging agent.

In an aspect, the cargo molecule is not fused to the fusion protein and is contained in the nanocage internally or wherein the cargo molecule is linked to the fusion protein or bound to the nanocage either internally or externally.

In an aspect, the cargo molecule is a protein and is fused to the fusion protein such that the cargo molecule is contained in the nanocage internally.

In an aspect, the cargo molecule comprises a fluorescent protein, such as GFP, EGFP, Ametrine, and/or a flavin-based fluorescent protein, such as a LOV-protein, such as iLOV.

In an aspect, the nanocage exhibits pan-virus neutralization breadth.

In an aspect, the nanocage exhibits an average median IC₅₀ value against a multiclade panel of 118 pseudoviruses (PsV) of less than about 0.1 ug/mL, such as less than about 0.01 ug/mL, such as less than about 0.001 ug/mL.

In an aspect, the nanocage exhibits an average median IC₅₀ value against a multiclade panel of 118 pseudoviruses (PsV) of less than about 42 pM, such as less than about 4.2 pM, such as less than about 0.42 pM.

In an aspect, the nanocage exhibits an average median IC₅₀ value against a multiclade panel of 118 pseudoviruses (PsV) that is at least about 10, at least about 100, at least about 1000, at least about 10,000, or at least about 100,000 more potent than a cocktail of the corresponding bNAbs on a mass and/or molar basis.

In accordance with an aspect, there is provided a fusion protein comprising a first nanocage monomer or a subunit thereof linked to an scFc fragment at the C-terminus of the first nanocage monomer or subunit thereof, wherein a plurality of the fusion proteins are capable of self-assembling to form a nanocage.

In an aspect, the subunit comprises an N-subunit or a C-subunit, corresponding substantially to the N-terminal half of a nanocage monomer and the C-terminal half of a nanocage monomer, respectively, wherein the N-subunit and the C-subunit are capable of self-assembling to form a nanocage monomer.

In an aspect, the scFc fragment is linked to the N-subunit or C-subunit at the C-terminus of the N-subunit or C-subunit.

In an aspect, the first nanocage monomer or subunit thereof is further linked to a first bioactive moiety.

In an aspect, the first bioactive moiety is linked to the first nanocage monomer or subunit thereof at the N- or C-terminus of the first nanocage monomer or subunit thereof, preferably the N-terminus.

In an aspect, the first bioactive moiety is linked to the N-subunit or C-subunit at the N-terminus of the N-subunit or C-subunit, preferably wherein the first bioactive moiety is linked to the C-subunit at the N-terminus.

In an aspect, the first bioactive moiety decorates the interior and/or exterior surface, preferably the exterior surface, of the assembled nanocage.

In an aspect, the first bioactive moiety comprises a first antigen-binding moiety.

In an aspect, the first antigen-binding moiety comprises an antibody or fragment thereof.

In an aspect, the first antigen-binding moiety comprises a Fab fragment.

In an aspect, the antibody or fragment thereof comprises a scFab fragment, a scFv fragment, a sdAb fragment, a VHH domain or a combination thereof.

In an aspect, the antibody or fragment thereof comprises a heavy and/or light chain of a Fab fragment.

In an aspect, the first antigen-binding moiety binds specifically to an antigen associated with an antibody-preventable and/or antibody-treatable condition.

In an aspect, the antigen is associated with an infectious agent, including a virus, bacteria, a parasite, a fungus, or a yeast, a cancer, or an immune disease, including an autoimmune disease.

In an aspect, the first antigen-binding moiety comprises an HIV-1 specific antigen-binding moiety.

In an aspect, the HIV-1 specific antigen-binding moiety binds to BG505 SOSIP_D368R, 93TH057 gp120, and/or an MPER peptide.

In an aspect, the HIV-1 specific antigen-binding moiety comprises an HIV-1 specific antigen-binding moiety from PGDM1400, 10E8v4, and/or N49P7.

In an aspect, the fusion protein comprises the first nanocage monomer or a subunit thereof linked to the scFc fragment at the C-terminus of the first nanocage monomer or a subunit thereof and linked to a first bioactive moiety at the N-terminus of the nanocage monomer or a subunit thereof.

In an aspect, the fusion protein comprises the C-subunit linked to the scFc fragment at the C-terminus of the C-subunit and linked to a first bioactive moiety at the N-terminus of the C-subunit.

In an aspect, the fusion protein is provided in combination with an N-subunit or a fusion protein comprising an N-subunit.

In an aspect, the N-subunit is linked to a second bioactive moiety at the N- or C-terminus, preferably the N-terminus.

In an aspect, the second bioactive moiety comprises a second antigen-binding moiety, and wherein, if the first bioactive moiety comprises the first antigen-binding moiety, the second antigen-binding moiety may be the same or different from the antigen-binding moiety that is linked to the C-subunit.

In an aspect, the second antigen-binding moiety comprises an antibody or fragment thereof.

In an aspect, the second antigen-binding moiety comprises a Fab fragment.

In an aspect, the antibody or fragment thereof comprises a scFab fragment, a scFv fragment, a sdAb fragment, a VHH domain or a combination thereof.

In an aspect, the antibody or fragment thereof comprises a heavy and/or light chain of a Fab fragment.

In an aspect, the N-subunit is further linked to a second scFc fragment at the N- or C-terminus of the N-subunit, preferably the C-terminus.

In an aspect, the scFc fragment is derived from an IgG, IgA, IgD, IgM, or IgE, and is preferably human.

In an aspect, the scFc fragment is derived from an IgG, such as IgG1, IgG2, IgG3, or IgG4.

In an aspect, the scFc fragment is an IgG1 scFc fragment.

In an aspect, the scFc fragment comprises one or more mutations or sets of mutations that modulate the half-life of the fusion protein from, for example, minutes or hours to several days, weeks, or months.

In an aspect, the scFc fragment comprises a mutation at one or more of L234, L235, G236, G237, M252, I253, S254, T256, P329, A330, M428, N434, or a combination thereof (wherein numbering is according to the EU index), such as M428L and N434S (“LS”); M252Y, S254T and T256E (“YTE”); L234A and L235A (“LALA”); I253A; L234A, L235A, and P329G (“LALAP”); G236R; G237A; and/or A330L or a combination thereof.

In an aspect, from about 3 to about 100 nanocage monomers, such as 24, 32, 48, or 60 nanocage monomers, or from about 4 to about 200 nanocage monomer subunits, such as 4, 6, 8, 10, 12, 14, 18, 20, 22, 24, 26, 28, 30, 32, 34, 36, 38, 40, 42, 44, 46, 48, 50, or more, optionally in combination with one or more whole nanocage monomers, are capable of self-assembling to form a nanocage.

In an aspect, the first nanocage monomer or subunit thereof is selected from ferritin, apoferritin, encapsulin, SOR, lumazine synthase, pyruvate dehydrogenase, carboxysome, vault proteins, GroEL, heat shock protein, E2P, MS2 coat protein, fragments thereof, and variants thereof.

In an aspect, the first nanocage monomer or subunit thereof is apoferritin, optionally human apoferritin.

In an aspect, the first nanocage monomer or subunit thereof is an apoferritin light chain, optionally human apoferritin light chain.

In an aspect, the fusion protein comprises a first apoferritin subunit, optionally a first human apoferritin subunit, and wherein the first apoferritin subunit is capable of self-assembling with a second apoferritin subunit.

In an aspect, the first and second apoferritin monomer subunits interchangeably comprise the “N” and “C” regions of apoferritin.

In an aspect, the “N” region of apoferritin comprises or consists of a sequence at least 70% (such as at least 75%, 80%, 85%, 90%, 95%, 96%, 97%, 98%, 99%, or 100%) identical to:

(SEQ ID NO: 1)
MSSQIRQNYSTDVEAAVNSLVNLYLQASYTYLSLGFYFDRDDVALEGVS
HFFRELAEEKREGYERLLKMQNQRGGRALFQDIKKPAEDEW
or
(SEQ ID NO: 15)
SSQIRQNYSTDVEAAVNSLVNLYLQASYTYLSLGFYFDRDDVALEGVSH
FFRELAEEKREGYERLLKMQNQRGGRALFQDIKKPAEDEW.

In an aspect, the “C” region of apoferritin comprises or consists of a sequence at least 70% (such as at least 75%, 80%, 85%, 90%, 95%, 96%, 97%, 98%, 99%, or 100%) identical to:

(SEQ ID NO: 2)
GKTPDAMKAAMALEKKLNQALLDLHALGSARTDPHLCDFLETHFLDEEV
KLIKKMGDHLTNLHRLGGPEAGLGEYLFERLTLRHD
or
(SEQ ID NO: 16)
GKTPDAMKAAMALEKKLNQALLDLHALGSARTDPHLCDFLETHFLDEEV
KLIKKMGDHLTNLHRLGGPEAGLGEYLFERLTLKHD.

In an aspect, the scFc fragment is linked to the first nanocage monomer or subunit thereof through a linker.

In an aspect, the first bioactive moiety is linked to the first nanocage monomer or subunit thereof through a linker.

In an aspect, the second bioactive moiety is linked to the N-subunit through a linker.

In an aspect, the linker is flexible or rigid and comprises from about 1 to about 100 amino acid residues, such as from about 1 to about 70 amino acid residues, such as from about 1 to about 30 amino acid residues, such as from about 8 to about 16 amino acid residues.

In an aspect, the linker comprises a GS domain.

In an aspect, the GS domain comprises a GS repeat, a GGS repeat, a GGGS (SEQ ID NO:11) repeat, and/or a GGGGS (SEQ ID NO:12) repeat, such as 1, 2, 3, 4, or more GGGGS (SEQ ID NO:12) repeats.

In an aspect, the linker comprises or consists of a sequence at least 70% (such as at least 75%, 80%, 85%, 90%, 95%, 96%, 97%, 98%, 99%, or 100%) identical to: GGGGSGGGGSGGGGSGGGGSGGGGSGG (SEQ ID NO:4).

(SEQ ID NO: 4)
GGGGSGGGGSGGGGSGGGGSGGGGSGG.

In an aspect, the linker comprises or consists of a sequence at least 70% (such as at least 75%, 80%, 85%, 90%, 95%, 96%, 97%, 98%, 99%, or 100%) identical to:

(SEQ ID NO: 5)
GGSGGSGGSGGSGGGSGGSGGSGGSG

In accordance with an aspect, there is provided a nanocage comprising at least one fusion protein as described herein and at least one second nanocage monomer or subunit thereof that self-assembles with the fusion protein.

In an aspect, the fusion protein comprises a first nanocage monomer subunit, the second nanocage monomer or subunit thereof is a second nanocage monomer subunit, and the second nanocage monomer subunit self-assembles with the fusion protein to form the nanocage monomer.

In an aspect, from about 1% to about 100%, such as from about 1%, 4%, 8%, 10%, 12%, 15%, 20%, 25%, 30%, 35%, 40%, 45%, 50%, 55%, 60%, 65%, 70%, 75%, 80%, 85%, 90%, or 95%, to about 4%, 8%, 10%, 12%, 15%, 20%, 25%, 30%, 35%, 40%, 45%, 50%, 55%, 60%, 65%, 70%, 75%, 80%, 85%, 90%, 95%, or 100%, such as from about 20% to about 80%, of the nanocage monomers or subunits thereof is comprised within the fusion protein described herein.

In an aspect, each nanocage monomer or subunit thereof is comprised within a fusion protein described herein.

In an aspect, the nanocage comprises 1 bioactive moiety or at least 2, 3, 4, 5, 6, 7, 8, 9, or 10 different bioactive moieties, such as 2 or 3 different bioactive moieties.

In an aspect, the nanocage is multivalent.

In an aspect, the nanocage is multispecific.

In an aspect, at least one bioactive moiety decorates the exterior surface of the nanocage and at least one scFc fragment decorates the exterior surface of the nanocage.

In an aspect, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 bioactive moieties decorate the exterior surface of the nanocage and at least two scFc fragments decorate the exterior surface of the nanocage.

In an aspect, the nanocage comprises:
(a) at least one nanocage monomer fused to a first antigen-binding moiety, such as a Fab,
(b) at least one N-subunit fused to a second antigen-binding moiety, such as a Fab, and
(c) and at least one C-subunit fused to:
   (i) a third antigen-binding moiety, such as a Fab at one terminus and
   (ii) a scFc fragment at the other terminus.

In an aspect, each antigen-binding moiety is linked to the N-terminus of the nanocage monomer or subunit thereof and wherein the scFc fragment is linked to the C-terminus of the C-subunit.

In an aspect, the first and/or second antigen-binding moiety binds specifically to an antigen associated with an antibody-preventable and/or antibody-treatable condition.

In an aspect, the antigen is associated with an infectious agent, including a virus, bacteria, a parasite, a fungus, or a yeast, a cancer, or an immune disease, including an autoimmune disease.

In an aspect, each antigen-binding moiety is a different HIV-1-specific Fab.

In an aspect, the HIV-1-specific Fab binds to BG505 SOSIP_D368R, 93TH057 gp120, and/or an MPER peptide.

In an aspect, the HIV-1 specific Fab comprises PGDM1400 Fab, 10E8v4 Fab, and/or N49P7 Fab.

In an aspect, the HIV-1 specific Fab comprises PGDM1400 Fab, 10E8v4 Fab, and N49P7 Fab.

In an aspect, the nanocage comprises at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 antigen-binding moieties.

In an aspect, the nanocage comprises at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 scFc fragments.

In an aspect, the nanocage is carrying a cargo molecule, such as a pharmaceutical agent, a diagnostic agent, and/or an imaging agent.

In an aspect, the cargo molecule is not fused to the fusion protein and is contained in the nanocage internally or wherein the cargo molecule is linked to the fusion protein or bound to the nanocage either internally or externally.

In an aspect, the cargo molecule is a protein and is fused to the fusion protein such that the cargo molecule is contained in the nanocage internally.

In an aspect, the cargo molecule comprises a fluorescent protein, such as GFP, EGFP, Ametrine, and/or a flavin-based fluorescent protein, such as a LOV-protein, such as iLOV.

In an aspect, the nanocage exhibits pan-virus neutralization breadth.

In an aspect, the nanocage exhibits an average median IC₅₀ value against a multiclade panel of 118 pseudoviruses (PsV) of less than about 0.1 ug/mL, such as less than about 0.01 ug/mL, such as less than about 0.001 ug/mL.

In an aspect, the nanocage exhibits an average median IC₅₀ value against a multiclade panel of 118 pseudoviruses (PsV) of less than about 42 pM, such as less than about 4.2 pM, such as less than about 0.42 pM.

In an aspect, the nanocage exhibits an average median IC₅₀ value against a multiclade panel of 118 pseudoviruses (PsV) that is at least about 10, at least about 100, at least about 1000, at least about 10,000, or at least about 100,000 more potent than a cocktail of the corresponding bNAbs on a mass and/or molar basis.

In an aspect, there is provided a therapeutic or prophylactic composition comprising the nanocage described herein.

In an aspect, the composition is for treating and/or preventing an antibody-preventable and/or antibody-treatable condition.

In an aspect, the antigen is associated with an infectious agent, including a virus, bacteria, a parasite, a fungus, or a yeast, a cancer, or an immune disease, including an autoimmune disease.

In an aspect, the composition is for treating and/or preventing an HIV-1-related condition.

In accordance with an aspect, there is provided a nucleic acid molecule encoding the fusion protein described herein.

In accordance with an aspect, there is provided a vector comprising the nucleic acid molecule described herein.

In accordance with an aspect, there is provided a host cell comprising the vector described herein and producing the fusion protein described herein.

In accordance with an aspect, there is provided a method for treating and/or preventing a condition, the method comprising administering the nanocage or the composition described herein to a subject in need thereof.

In an aspect, the condition is an HIV-1-related condition.

In accordance with an aspect, there is provided a use of the nanocage or the composition described herein for treating and/or preventing a condition.

In an aspect, the condition is an HIV-1-related condition.

In accordance with an aspect, there is provided the nanocage or the composition described herein for use in treating and/or preventing a condition.

In an aspect, the condition is an HIV-1-related condition.

In an aspect, the nanocage does not include any ferritin heavy chains.

In an aspect, the nanocage does not include any components capable of ferroxidase activity.

In an aspect, the nanocage comprises at least one bioactive moiety comprising an antibody or antigen-binding fragment thereof, and the ratio of the total number of bioactive moieties to the number of Fc dimers is 6:1.

In an aspect, the nanocage comprises at least one bioactive moiety comprising an antibody or antigen-binding fragment thereof, and the ratio of the total number of bioactive moieties to the number of scFcs is 3:1.

In an aspect, the nanocage comprises at least one bioactive moiety comprising an antibody or antigen-binding fragment thereof, and the ratio of the total number of bioactive moieties to the number of Fc dimers or scFcs is at least 7:1.

In an aspect, the nanocage comprises at least one bioactive moiety comprising an antibody or antigen-binding fragment thereof, and the ratio of the total number of bioactive moieties to the number of Fc dimers or scFcs is at least 4:1.

In accordance with an aspect, there is provided a fusion protein comprising a nanocage monomer or a subunit thereof linked to an Fc monomer, wherein a plurality of the fusion proteins self-assemble to form a nanocage comprising one or more Fc dimers.

In an aspect, the subunit comprises an N-subunit or a C-subunit, corresponding substantially to the N-terminal half of the nanocage monomer and the C-terminal half of the nanocage monomer, respectively, wherein the N-subunit and the C-subunit are capable of self-assembling to form the nanocage monomer.

In an aspect, the Fc monomer is linked to the nanocage monomer or subunit thereof at the N- or C-terminus of the nanocage monomer or subunit thereof, preferably at the C-terminus.

In an aspect, the Fc monomer is linked to the N-subunit or C-subunit at the C-terminus of the N-subunit or C-subunit, preferably wherein the Fc monomer is linked to the C-subunit at the C-terminus.

In an aspect, the nanocage monomer or subunit thereof is further linked to a bioactive moiety.

In an aspect, the bioactive moiety is linked to the nanocage monomer or subunit thereof at the N- or C-terminus of the nanocage monomer or subunit thereof, preferably the N-terminus.

In an aspect, the bioactive moiety is linked to the N-subunit or C-subunit at the N-terminus of the N-subunit or C-subunit, preferably wherein the bioactive moiety is linked to the C-subunit at the N-terminus.

In an aspect, the antigen-binding moiety decorates the interior and/or exterior surface, preferably the exterior surface, of the assembled nanocage.

In an aspect, the bioactive moiety comprises an antigen-binding moiety.

In an aspect, the antigen-binding moiety comprises an antibody or fragment thereof.

In an aspect, the antigen-binding moiety comprises a Fab fragment.

In an aspect, the antibody or fragment thereof comprises a scFab fragment, a scFv fragment, a sdAb fragment, a VHH domain or a combination thereof.

In an aspect, the antibody or fragment thereof comprises a heavy and/or light chain of a Fab fragment.

In an aspect, the antigen-binding moiety binds specifically to an antigen associated with an antibody-preventable and/or antibody-treatable condition.

In an aspect, the antigen is associated with an infectious agent, including a virus, bacteria, a parasite, a fungus, or a yeast, a cancer, or an immune disease, including an autoimmune disease.

In an aspect, the antigen-binding moiety comprises an HIV-1 antigen-binding moiety.

In an aspect, the HIV-1-specific antigen-binding moiety binds to BG505 SOSIP D368R, 93TH057 gp120, and/or an MPER peptide.

In an aspect, the HIV-1 antigen-binding moiety comprises PGDM1400, 10E8v4, and/or N49P7.

In an aspect, the fusion protein comprises the nanocage monomer linked to the Fc monomer at the C-terminus of the nanocage monomer and linked to the bioactive moiety at the N-terminus of the nanocage monomer.

In an aspect, the fusion protein comprises the C-subunit linked to the Fc monomer at the C-terminus of the C-subunit and linked to the bioactive moiety at the N-terminus of the C-subunit.

In an aspect, the C-subunit is provided in combination with the N-subunit.

In an aspect, the N-subunit is linked to a bioactive moiety at the N- or C-terminus, preferably the N-terminus.

In an aspect, the bioactive moiety comprises an antigen-binding moiety, which may be the same or different from the antigen-binding moiety that is linked to the C-subunit.

In an aspect, the antigen-binding moiety comprises an antibody or fragment thereof.

In an aspect, the antigen-binding moiety comprises a Fab fragment.

In an aspect, the antibody or fragment thereof comprises a scFab fragment, a scFv fragment, a sdAb fragment, a VHH domain or a combination thereof.

In an aspect, the antibody or fragment thereof comprises a heavy and/or light chain of a Fab fragment.

In an aspect, the N-subunit is further linked to an Fc monomer at the N- or C-terminus of the N-subunit, preferably the C-terminus.

In an aspect, the Fc monomer is derived from an IgG, IgA, IgD, IgM, or IgE, and is preferably human.

In an aspect, the Fc monomer is derived from an IgG, such as IgG1, IgG2, IgG3, or IgG4.

In an aspect, the Fc monomer is an IgG1 Fc monomer.

In an aspect, the Fc monomer comprises one or more mutations or sets of mutations that modulate the half-life of the fusion protein from, for example, minutes or hours to several days, weeks, or months.

In an aspect, the Fc monomer comprises a mutation at one or more of L234, L235, G236, G237, M252, I253, S254, T256, P329, A330, M428, N434, or a combination thereof (wherein numbering is according to the EU index), such as M428L and N434S (“LS”); M252Y, S254T and T256E (“YTE”); L234A and L235A (“LALA”); I253A, and/or L234A, L235A, and P329G (“LALAP”), G236R, G237A, A330L or a combination thereof.

In an aspect, from about 3 to about 100 nanocage monomers, such as 24, 32, 48, or 60 monomers, or from about 4 to about 200 nanocage monomer subunits, such as 4, 6, 8, 10, 12, 14, 18, 20, 22, 24, 26, 28, 30, 32, 34, 36, 38, 40, 42, 44, 46, 48, 50, or more, optionally in combination with one or more whole nanocage monomers, are capable of self-assembling to form a nanocage.

In an aspect, the nanocage monomer or subunit thereof is selected from ferritin, apoferritin, encapsulin, SOR, lumazine synthase, pyruvate dehydrogenase, carboxysome, vault proteins, GroEL, heat shock protein, E2P, MS2 coat protein, fragments thereof, and variants thereof.

In an aspect, the nanocage monomer or subunit thereof is apoferritin, optionally human apoferritin.

In an aspect, the nanocage monomer or subunit thereof is an apoferritin light chain, optionally human apoferritin light chain.

In an aspect, the fusion protein comprises a first apoferritin subunit, optionally a first human apoferritin subunit, and wherein the first apoferritin subunit is capable of self-assembling with a second apoferritin subunit.

In an aspect, the first and second apoferritin monomer subunits interchangeably comprise the “N” and “C” regions of apoferritin.

In an aspect, the “N” region of apoferritin comprises or consists of a sequence at least 70% (such as at least 75%, 80%, 85%, 90%, 95%, 96%, 97%, 98%, 99%, or 100%) identical to:

MSSQIRQNYSTDVEAAVNSLVNLYLQASYTYLSLGFYFDRDDVALEGVS
HFFRELAEEKREGYERLLKMQNQRGGRALFQDIKKPAEDEW.

In an aspect, the “C” region of apoferritin comprises or consists of a sequence at least 70% (such as at least 75%, 80%, 85%, 90%, 95%, 96%, 97%, 98%, 99%, or 100%) identical to:

GKTPDAMKAAMALEKKLNQALLDLHALGSARTDPHLCDFLETHFLDEEV
KLIKKMGDHLTNLHRLGGPEAGLGEYLFERLTLRHD.

In an aspect, the Fc monomer and/or the bioactive moiety is linked to the nanocage monomer or subunit thereof through a linker.

In an aspect, the linker is flexible or rigid and comprises from about 1 to about 100 amino acid residues, such as from about 1 to about 70 amino acid residues, such as from about 1 to about 30 amino acid residues, such as from about 8 to about 16 amino acid residues.

In an aspect, the linker comprises a GS domain.

In an aspect, the GS domain comprises a GS repeat, a GGS repeat, a GGGS repeat, and/or a GGGGS repeat, such as 1, 2, 3, 4, or more GGGGS repeats.

In an aspect, the linker comprises or consists of a sequence at least 70% (such as at least 75%, 80%, 85%, 90%, 95%, 96%, 97%, 98%, 99%, or 100%) identical to: GGGGSGGGGSGGGGSGGGGSGGGGSGG.

GGGGSGGGGSGGGGSGGGGSGGGGSGG.

In an aspect, the linker comprises or consists of a sequence at least 70% (such as at least 75%, 80%, 85%, 90%, 95%, 96%, 97%, 98%, 99%, or 100%) identical to:

GGSGGSGGSGGSGGGSGGSGGSGGSG.

In accordance with an aspect, there is provided a nanocage comprising at least one fusion protein described herein and at least one second nanocage monomer or subunit thereof that self-assembles with the fusion protein.

In an aspect, the fusion protein comprises a first nanocage monomer subunit, the second nanocage monomer or subunit thereof is a second nanocage monomer subunit, and the second nanocage monomer subunit self-assembles with the fusion protein to form the nanocage monomer.

In an aspect, each nanocage monomer comprises a fusion protein described herein.

In an aspect, from about 1% to about 100%, such as from about 1%, 4%, 8%, 10%, 12%, 15%, 20%, 25%, 30%, 35%, 40%, 45%, 50%, 55%, 60%, 65%, 70%, 75%, 80%, 85%, 90%, or 95%, to about 4%, 8%, 10%, 12%, 15%, 20%, 25%, 30%, 35%, 40%, 45%, 50%, 55%, 60%, 65%, 70%, 75%, 80%, 85%, 90%, 95%, or 100%, such as from about 20% to about 80%, of the nanocage monomers or subunits thereof comprise the fusion protein described herein.

In an aspect, the nanocage comprises 1 bioactive moiety or at least 2, 3, 4, 5, 6, 7, 8, 9, or 10 different bioactive moieties, such as 2 or 3 different bioactive moieties.

In an aspect, the nanocage is multivalent.

In an aspect, the nanocage is multispecific.

In an aspect, at least one bioactive moiety decorates the exterior surface of the nanocage and at least one Fc dimer decorates the exterior surface of the nanocage.

In an aspect, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 bioactive moieties decorate the exterior surface of the nanocage and at least two Fc dimers decorate the exterior surface of the nanocage.

In an aspect, the nanocage comprises at least one nanocage monomer fused to a first antigen-binding moiety, such as a Fab, at least one N-subunit fused to a second antigen-binding moiety, such as a Fab, and at least one C-subunit fused to a third antigen-binding moiety, such as a Fab at one terminus and a Fc monomer at the other terminus.

In an aspect, each antigen-binding moiety is linked to the N-terminus of the nanocage monomer or subunit thereof and wherein the Fc monomer is linked to the C-terminus of the C-subunit.

In an aspect, the antigen-binding moiety binds specifically to an antigen associated with an antibody-preventable and/or antibody-treatable condition.

In an aspect, the antigen is associated with an infectious agent, including a virus, bacteria, a parasite, a fungus, or a yeast, a cancer, or an immune disease, including an autoimmune disease.

In an aspect, each antigen-binding moiety is a different HIV-1-specific Fab.

In an aspect, the HIV-1-specific Fab binds to BG505 SOSIP D368R, 93TH057 gp120, and/or an MPER peptide.

In an aspect, the HIV-1 specific Fab comprises PGDM1400, 10E8v4, and/or N49P7.

In an aspect, the nanocage comprises PGDM1400, 10E8v4, and N49P7.

In an aspect, the nanocage comprises at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 antigen-binding moieties.

In an aspect, the nanocage comprises at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 Fc monomers.

In an aspect, the nanocage is carrying a cargo molecule, such as a pharmaceutical agent, a diagnostic agent, and/or an imaging agent.

In an aspect, the cargo molecule is not fused to the fusion protein and is contained in the nanocage internally or wherein the cargo molecule is linked to the fusion protein or bound to the nanocage either internally or externally.

In an aspect, the cargo molecule is a protein and is fused to the fusion protein such that the cargo molecule is contained in the nanocage internally.

In an aspect, the cargo molecule comprises a fluorescent protein, such as GFP, EGFP, Ametrine, and/or a flavin-based fluorescent protein, such as a LOV-protein, such as iLOV.

In an aspect, the nanocage exhibits pan-virus neutralization breadth.

In an aspect, the nanocage exhibits an average median IC₅₀ value against a multiclade panel of 118 pseudoviruses (PsV) of less than about 0.1 ug/mL, such as less than about 0.01 ug/mL, such as less than about 0.001 ug/mL.

In an aspect, the nanocage exhibits an average median IC₅₀ value against a multiclade panel of 118 pseudoviruses (PsV) of less than about 42 pM, such as less than about 4.2 pM, such as less than about 0.42 pM.

In an aspect, the nanocage exhibits an average median IC₅₀ value against a multiclade panel of 118 pseudoviruses (PsV) that is at least about 10, at least about 100, at least about 1000, at least about 10,000, or at least about 100,000 more potent than a cocktail of the corresponding bNAbs on a mass and/or molar basis.

In accordance with an aspect, there is provided a fusion protein comprising a nanocage monomer or a subunit thereof linked to an scFc fragment at the C-terminus of the nanocage monomer or subunit thereof, wherein a plurality of the fusion proteins self-assemble to form a nanocage.

In an aspect, the subunit comprises an N-subunit or a C-subunit, corresponding substantially to the N-terminal half of the nanocage monomer and the C-terminal half of the nanocage monomer, respectively, wherein the N-subunit and the C-subunit are capable of self-assembling to form the nanocage monomer.

In an aspect, the scFc fragment is linked to the nanocage monomer at the C-terminus of the nanocage monomer.

In an aspect, the scFc fragment is linked to the N-subunit or C-subunit at the C-terminus of the N-subunit or C-subunit.

In an aspect, the nanocage monomer or subunit thereof is further linked to a bioactive moiety.

In an aspect, the bioactive moiety is linked to the nanocage monomer or subunit thereof at the N- or C-terminus of the nanocage monomer or subunit thereof, preferably the N-terminus.

In an aspect, the bioactive moiety is linked to the N-subunit or C-subunit at the N-terminus of the N-subunit or C-subunit, preferably wherein the bioactive moiety is linked to the C-subunit at the N-terminus.

In an aspect, the antigen-binding moiety decorates the interior and/or exterior surface, preferably the exterior surface, of the assembled nanocage.

In an aspect, the bioactive moiety comprises an antigen-binding moiety.

In an aspect, the antigen-binding moiety comprises an antibody or fragment thereof.

In an aspect, the antigen-binding moiety comprises a Fab fragment.

In an aspect, the antibody or fragment thereof comprises a scFab fragment, a scFv fragment, a sdAb fragment, a VHH domain or a combination thereof.

In an aspect, the antibody or fragment thereof comprises a heavy and/or light chain of a Fab fragment.

In an aspect, the antigen-binding moiety binds specifically to an antigen associated with an antibody-preventable and/or antibody-treatable condition.

In an aspect, the antigen is associated with an infectious agent, including a virus, bacteria, a parasite, a fungus, or a yeast, a cancer, or an immune disease, including an autoimmune disease.

In an aspect, the antigen-binding moiety comprises an HIV-1 antigen-binding moiety.

In an aspect, the HIV-1-specific antigen-binding moiety binds to BG505 SOSIP D368R, 93TH057 gp120, and/or an MPER peptide.

In an aspect, the HIV-1 antigen-binding moiety comprises PGDM1400, 10E8v4, and/or N49P7.

In an aspect, the fusion protein comprises the nanocage monomer linked to the scFc fragment at the C-terminus of the nanocage monomer and linked to the bioactive moiety at the N-terminus of the nanocage monomer.

In an aspect, the fusion protein comprises the C-subunit linked to the scFc fragment at the C-terminus of the C-subunit and linked to the bioactive moiety at the N-terminus of the C-subunit.

In an aspect, the C-subunit is provided in combination with the N-subunit.

In an aspect, the N-subunit is linked to a bioactive moiety at the N- or C-terminus, preferably the N-terminus.

In an aspect, the bioactive moiety comprises an antigen-binding moiety, which may be the same or different from the antigen-binding moiety that is linked to the C-subunit.

In an aspect, the antigen-binding moiety comprises an antibody or fragment thereof.

In an aspect, the antigen-binding moiety comprises a Fab fragment.

In an aspect, the antibody or fragment thereof comprises a scFab fragment, a scFv fragment, a sdAb fragment, a VHH domain or a combination thereof.

In an aspect, the antibody or fragment thereof comprises a heavy and/or light chain of a Fab fragment.

In an aspect, the N-subunit is further linked to an scFc fragment at the N- or C-terminus of the N-subunit, preferably the C-terminus.

In an aspect, the scFc fragment is derived from an IgG, IgA, IgD, IgM, or IgE, and is preferably human.

In an aspect, the scFc fragment is derived from an IgG, such as IgG1, IgG2, IgG3, or IgG4.

In an aspect, the scFc fragment is an IgG1 scFc fragment.

In an aspect, the scFc fragment comprises one or more mutations or sets of mutations that modulate the half-life of the fusion protein from, for example, minutes or hours to several days, weeks, or months.

In an aspect, the scFc fragment comprises a mutation at one or more of L234, L235, G236, G237, M252, I253, S254, T256, P329, A330, M428, N434, or a combination thereof (wherein numbering is according to the EU index), such as M428L and N434S (“LS”); M252Y, S254T and T256E (“YTE”); L234A and L235A (“LALA”); I253A, and/or L234A, L235A, and P329G (“LALAP”), G236R, G237A, A330L or a combination thereof.

In an aspect, from about 3 to about 100 nanocage monomers, such as 24, 32, 48, or 60 monomers, or from about 4 to about 200 nanocage monomer subunits, such as 4, 6, 8, 10, 12, 14, 18, 20, 22, 24, 26, 28, 30, 32, 34, 36, 38, 40, 42, 44, 46, 48, 50, or more, optionally in combination with one or more whole nanocage monomers, are capable of self-assembling to form a nanocage.

In an aspect, the nanocage monomer or subunit thereof is selected from ferritin, apoferritin, encapsulin, SOR, lumazine synthase, pyruvate dehydrogenase, carboxysome, vault proteins, GroEL, heat shock protein, E2P, MS2 coat protein, fragments thereof, and variants thereof.

In an aspect, the nanocage monomer or subunit thereof is apoferritin, optionally human apoferritin.

In an aspect, the nanocage monomer or subunit thereof is an apoferritin light chain, optionally human apoferritin light chain.

In an aspect, the fusion protein comprises a first apoferritin subunit, optionally a first human apoferritin subunit, and wherein the first apoferritin subunit is capable of self-assembling with a second apoferritin subunit.

In an aspect, the first and second apoferritin monomer subunits interchangeably comprise the “N” and “C” regions of apoferritin.

In an aspect, the “N” region of apoferritin comprises or consists of a sequence at least 70% (such as at least 75%, 80%, 85%, 90%, 95%, 96%, 97%, 98%, 99%, or 100%) identical to:

MSSQIRQNYSTDVEAAVNSLVNLYLQASYTYLSLGFYFDRDDVALEGVS
HFFRELAEEKREGYERLLKMQNQRGGRALFQDIKKPAEDEW.

In an aspect, the “C” region of apoferritin comprises or consists of a sequence at least 70% (such as at least 75%, 80%, 85%, 90%, 95%, 96%, 97%, 98%, 99%, or 100%) identical to:

GKTPDAMKAAMALEKKLNQALLDLHALGSARTDPHLCDFLETHFLDEEV
KLIKKMGDHLTNLHRLGGPEAGLGEYLFERLTLRHD.

In an aspect, the scFc fragment and/or the bioactive moiety is linked to the nanocage monomer or subunit thereof through a linker.

In an aspect, the linker is flexible or rigid and comprises from about 1 to about 100 amino acid residues, such as from about 1 to about 70 amino acid residues, such as from about 1 to about 30 amino acid residues, such as from about 8 to about 16 amino acid residues.

In an aspect, the linker comprises a GS domain.

In an aspect, the GS domain comprises a GS repeat, a GGS repeat, a GGGS repeat, and/or a GGGGS repeat, such as 1, 2, 3, 4, or more GGGGS repeats.

In an aspect, the linker comprises or consists of a sequence at least 70% (such as at least 75%, 80%, 85%, 90%, 95%, 96%, 97%, 98%, 99%, or 100%) identical to: GGGGSGGGGSGGGGSGGGGSGGGGSGG.

GGGGSGGGGSGGGGSGGGGSGGGGSGG.

In an aspect, the linker comprises or consists of a sequence at least 70% (such as at least 75%, 80%, 85%, 90%, 95%, 96%, 97%, 98%, 99%, or 100%) identical to:

GGSGGSGGSGGSGGGSGGSGGSGGSG

In accordance with an aspect, there is provided a nanocage comprising at least one fusion protein described herein and at least one second nanocage monomer or subunit thereof that self-assembles with the fusion protein.

In an aspect, the fusion protein comprises a first nanocage monomer subunit, the second nanocage monomer or subunit thereof is a second nanocage monomer subunit, and the second nanocage monomer subunit self-assembles with the fusion protein to form the nanocage monomer.

In an aspect, each nanocage monomer comprises the fusion protein described herein.

In an aspect, from about 1% to about 100%, such as from about 1%, 4%, 8%, 10%, 12%, 15%, 20%, 25%, 30%, 35%, 40%, 45%, 50%, 55%, 60%, 65%, 70%, 75%, 80%, 85%, 90%, or 95%, to about 4%, 8%, 10%, 12%, 15%, 20%, 25%, 30%, 35%, 40%, 45%, 50%, 55%, 60%, 65%, 70%, 75%, 80%, 85%, 90%, 95%, or 100%, such as from about 20% to about 80%, of the nanocage monomers or subunits thereof comprise the fusion protein described herein.

In an aspect, the nanocage comprises 1 bioactive moiety or at least 2, 3, 4, 5, 6, 7, 8, 9, or 10 different bioactive moieties, such as 2 or 3 different bioactive moieties.

In an aspect, the nanocage is multivalent.

In an aspect, the nanocage is multispecific.

In an aspect, at least one bioactive moiety decorates the exterior surface of the nanocage and at least one scFc fragment decorates the exterior surface of the nanocage.

In an aspect, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 bioactive moieties decorate the exterior surface of the nanocage and at least two scFc fragments decorate the exterior surface of the nanocage.

In an aspect, the nanocage comprises at least one nanocage monomer fused to a first antigen-binding moiety, such as a scFab, at least one N-subunit fused to a second antigen-binding moiety, such as a scFab, and at least one C-subunit fused to a third antigen-binding moiety, such as a scFab at one terminus and a scFc fragment at the other terminus.

In an aspect, each antigen-binding moiety is linked to the N-terminus of the nanocage monomer or subunit thereof and wherein the scFc fragment is linked to the C-terminus of the C-subunit.

In an aspect, the antigen-binding moiety binds specifically to an antigen associated with an antibody-preventable and/or antibody-treatable condition.

In an aspect, the antigen is associated with an infectious agent, including a virus, bacteria, a parasite, a fungus, or a yeast, a cancer, or an immune disease, including an autoimmune disease.

In an aspect, each antigen-binding moiety is a different HIV-1-specific Fab.

In an aspect, the HIV-1-specific Fab binds to BG505 SOSIP D368R, 93TH057 gp120, and/or an MPER peptide.

In an aspect, the HIV-1 specific Fab comprises PGDM1400, 10E8v4, and/or N49P7.

In an aspect, the nanocage comprises PGDM1400, 10E8v4, and N49P7.

In an aspect, the nanocage comprises at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 antigen-binding moieties.

In an aspect, the nanocage comprises at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 Fc monomers.

In an aspect, the nanocage is carrying a cargo molecule, such as a pharmaceutical agent, a diagnostic agent, and/or an imaging agent.

In an aspect, the cargo molecule is not fused to the fusion protein and is contained in the nanocage internally or wherein the cargo molecule is linked to the fusion protein or bound to the nanocage either internally or externally.

In an aspect, the cargo molecule is a protein and is fused to the fusion protein such that the cargo molecule is contained in the nanocage internally.

In an aspect, the cargo molecule comprises a fluorescent protein, such as GFP, EGFP, Ametrine, and/or a flavin-based fluorescent protein, such as a LOV-protein, such as iLOV.

In an aspect, the nanocage exhibits pan-virus neutralization breadth.

In an aspect, the nanocage exhibits an average median IC₅₀ value against a multiclade panel of 118 pseudoviruses (PsV) of less than about 0.1 ug/mL, such as less than about 0.01 ug/mL, such as less than about 0.001 ug/mL.

In an aspect, the nanocage exhibits an average median IC₅₀ value against a multiclade panel of 118 pseudoviruses (PsV) of less than about 42 pM, such as less than about 4.2 pM, such as less than about 0.42 pM.

In an aspect, the nanocage exhibits an average median IC₅₀ value against a multiclade panel of 118 pseudoviruses (PsV) that is at least about 10, at least about 100, at least about 1000, at least about 10,000, or at least about 100,000 more potent than a cocktail of the corresponding bNAbs on a mass and/or molar basis.

In accordance with an aspect, there is provided a therapeutic or prophylactic composition comprising the nanocage described herein.

In an aspect, the composition is for treating and/or preventing an antibody-preventable and/or antibody-treatable condition.

In an aspect, the antigen is associated with an infectious agent, including a virus, bacteria, a parasite, a fungus, or a yeast, a cancer, or an immune disease, including an autoimmune disease.

In an aspect, the composition is for treating and/or preventing HIV-1.

In accordance with an aspect, there is provided a nucleic acid molecule encoding the fusion protein described herein.

In accordance with an aspect, there is provided a vector comprising the nucleic acid molecule described herein.

In accordance with an aspect, there is provided a host cell comprising the vector described herein and producing the fusion protein described herein.

In accordance with an aspect, there is provided a method for treating and/or preventing a condition, the method comprising administering the nanocage or the composition described herein.

In an aspect, the condition is HIV-1.

In accordance with an aspect, there is provided a use of the nanocage or the composition described herein for treating and/or preventing a condition.

In an aspect, the condition is HIV-1.

In an aspect, the use is for treating and/or preventing a condition.

In an aspect, the condition is HIV-1.

The novel features of the present invention will become apparent to those of skill in the art upon examination of the following detailed description of the invention. It should be understood, however, that the detailed description of the invention and the specific examples presented, while indicating certain aspects of the present invention, are provided for illustration purposes only because various changes and modifications within the spirit and scope of the invention will become apparent to those of skill in the art from the detailed description of the invention and claims that follow.

### BRIEF DESCRIPTION OF THE DRAWINGS

The present invention will be further understood from the following description with reference to the Figures, in which:

FIG. 1. HIV-1 bNAb multimerization drives increases in neutralization potency. Schematic of the self-assembly of (a) apoferritin (24 subunits) and (b) single-chain Fab-apoferritin fusions. Fab light chain (LC) and heavy chain (HC) are shown in light and dark pink, respectively, and are connected to the N terminus of the light chain of human apoferritin (grey) through a GGS-like flexible linker (dark). (c) Schematic representation of different Fab densities displayed on human apoferritin. Co-transfection of scFab-human apoferritin-encoding plasmids together with unconjugated apoferritin at ratios of 1:4 (dark yellow), 1:1 (black), 4:1 (blue) and 1:0 (red) resulted in molecules with different scFab valency, as confirmed by earlier elution volumes and less unconjugated apoferritin in size exclusion chromatography and SDS-PAGE. Negative stain electron micrographs of the samples with the lowest and highest valency are shown. (d) Avidity effect for neutralization of five bNAbs against a five-PsV panel (PVO.04, JRCSF, BG505 T332N, THRO4156.18 and t278-50). IC₅₀-fold increase analysis was omitted in the following cases: N49P7-t278-50, VRC01-T278-50 and 10-1074-THRO4156.18 due to the neutralization resistance. Potency fold increase was calculated as the parental IgG IC₅₀ (μg/mL) divided by the Fab-apoferritin fusion IC₅₀ (μg/mL). Bars (±SD) represent the mean value from n=3 biologically independent samples.

FIG. 2. Characterization of scFab-apoferritin fusions. (a) SDS-PAGE bands corresponding to scFab-apoferritin and unconjugated apoferritin were quantified by densitometry using the ImageJ software (rsb.info.nih.gov/iil/). Intensity plots of the bands in each lane (yellow box) are shown (b). (c) The approximate number of scFabs displayed on the particles was calculated as follows: intensity of scFab band/total intensity, and compared with the theoretical numbers inferred from DNA ratios used for co-transfection.

FIG. 3. Design, assembly and biophysical characterization of 32-N Multabodies. (a) Schematic for the human apoferritin split design that favors hetero-dimerization of scFab-human apoferritin subunits. Based on the necessary hetero-oligomerization to drive self-assembly, purification of Multabodies with the four components can be achieved by a two-step purification: protein A (Fc binding) and protein L (PGDM1400 binding). (b) Negative stain electron micrographs of 32-N. (c) Size exclusion chromatography in-line with multi-angle light scattering of 24-mer PGDM1400 (black) and 32-N Multabody (dark magenta). The molar mass of each elution peak (lines under UV absorbance) shows the sample is monodisperse, and that the Multabody is bigger in size than the 24-mer format due to additional antibody fragments in this design. (d) Comparison on the Tₘ and T_{agg} temperatures of 32-N, 12-mer multabodies and parental IgGs. (e) Concentration-response curves for binding of the 32-N Multabody to multiple epitopes. PGDM1400, N49P7 and 10E8v4 binding sites are colored in red, blue and pink, respectively in the surface representation of the HIV Env (in grey). Functional binding of Fc to human FcRn was tested by measuring binding at pH 7.5 and pH 5.6. The BG505 SOSIP.664_D368R trimer and 93TH057 gp120 monomer were selected as epitope-specific ligands for PGDM1400 and N49P7, respectively.

FIG. 4. Binding profile of bNAbs PGDM1400, 10E8v4 and N49P7. BLI response curves of IgG binding to 93TH057 gp120, BG505 SOSIP.664_D368R and MPER His-tagged antigens immobilized onto Ni-NTA biosensors.

FIG. 5. Neutralization properties of 32-N multabodies against a 14-pseudovirus panel. (a) Breadth and median IC₅₀ values (μg/mL) of the 32-N Multabody (red diamond), parental bNAbs (black circles) and IgG combination (blue triangle). The IgG cocktails contained each of the parental antibodies as in the Multabody sample (i.e. PGDM1400, N497 and 10E8v4). The 14-PsV panel was selected based on susceptibility and resistance to the parental IgGs. (b) IC₅₀ values (μg/mL) to each PsV variant.

FIG. 6. Schematic representation of the second generation Multabody version 2 (MB.v2; see bottom of FIG. 6). The single-chain Fc region (green) was fused to the C-terminus of the second half of apoferritin in the split Multabody design. This modification inverts the orientation of the Fc in the Multabody with respect to the orientation adopted in the previous version (MB.v1; see top of FIG. 6).

FIG. 7. Schematic of the second generation Multabody version 3 (MB.v3). (a) The single-chain Fc region (green), fused to the C-terminus of an apoferritin protomer (specifically to the C-half of ferritin) was reverted to a monomeric Fc chain. (b) Folding of a functional Fc fragment results from the homodimerization of two Fc chains fused to the C-terminus of two independent ferritin subunits at the 4-fold symmetry axes of the apoferritin nanocage.

FIG. 8. Characterization of the optimized Multabody versions. (a) Negative stain electron micrographs of 32-N MB.v2 and 32-N MB.v3. Comparison on the T_{agg} temperatures (b), and stability over time under stress condition of temperature (c) of the three different versions. Functional neutralization assay to assess active Multabody before and after the accelerated thermostability assay. Concentration-response curves for binding to multiple epitopes (d), and Fc receptors (e-f). 32-N MB.v3 with the Fc half-life extension mutation LS was incorporated in the assay.

FIG. 9. Neutralization properties of 32-N Multabodies versions 2 and 3. (a) Breadth and median IC₅₀ values (μg/mL) comparison of the 32-N Multabody versions (different shades of red diamond) and the IgG mixture (blue triangle) against a 25-PsV panel with 44% of PsV variants resistant to PGDM1400 neutralization. IC₅₀ values (μg/mL) to each PsV are shown. (b) Potency-Breadth curves comparing in gram and molar amount the three 32-N Multabody versions as well as the parental IgGs and an IgG mixture against an extended multiclade panel of 118 PsV.

FIG. 10. In vivo exposure of 32-N Multabody v3 in immunodeficient mice. Three female NOD/Shi-scid/lL-2Rγnull immunodeficient mouse strain (NCG) mice per group were used to assess the Multabody circulation in blood after subcutaneous administration of 5 mg/kg of 32-N MB.v3 with a Fc-modified (LS mutation) and parental IgG mixture for comparison.

### DETAILED DESCRIPTION OF CERTAIN ASPECTS

Described herein are nanocage platforms formed from a plurality of self-assembling nanocage monomers. These are termed “Multabody” platforms and allow modulation of binding and pharmacokinetic features of the nanocages, e.g., by controlling the number or ratio of fused molecules within nanocages.

Each monomer of the nanocage may be independently expressed as-is or may be fused to another moiety, such as an scFc fragment or an Fc monomer. Bioactive moieties, such as antibodies or fragments thereof, such as Fab fragments, may also be fused to one or more or all of the monomers in order to yield a nanocage that is multivalent and/or multispecific. Furthermore, each monomer may be independently split into subunits, such that each subunit comprises about one half of a monomer, providing an additional level or control and source of display for the scFc fragments, Fc monomers, and/or bioactive moieties.

Here, it is demonstrated that multimerization of HIV-1-targeting Fabs on the Multabody platform in combination with Fc monomers or scFc fragments results in a significantly greater ability to neutralize HIV-1 virus. As demonstrated herein, the increased potency and pan-neutralization ability of the Multabodies is not simply increased at a 1:1 ratio, where a doubling in valency results in a doubling in potency. Instead, the potency is, in aspects, synergistically increased by at least 10-fold and in aspects much more. The therapeutic potential of this engineered molecule was demonstrated using a 25-PsV panel that contains 44% of PsV variants resistant to the Fab specificity that leads MB potency and against an extended multiclade panel of 118 PsV.

### Definitions

Unless otherwise explained, all technical and scientific terms used herein have the same meaning as commonly understood by one of ordinary skill in the art to which this disclosure belongs. Definitions of common terms in molecular biology may be found in, e.g., Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). Although any methods and materials similar or equivalent to those described herein can be used in the practice for testing of the present invention, the typical materials and methods are described herein. In describing and claiming the present invention, the following terminology will be used.

It is also to be understood that the terminology used herein is for the purpose of describing particular aspects only, and is not intended to be limiting. Many patent applications, patents, and publications are referred to herein to assist in understanding the aspects described. Each of these references are incorporated herein by reference in their entirety.

In understanding the scope of the present application, the articles “a”, “an”, “the”, and “said” are intended to mean that there are one or more of the elements. Additionally, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

It will be understood that any aspects described as “comprising” certain components may also “consist of” or “consist essentially of,” wherein “consisting of” has a closed-ended or restrictive meaning and “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like. Typically, a composition consisting essentially of a set of components will comprise less than 5% by weight, typically less than 3% by weight, more typically less than 1%, and even more typically less than 0.1% by weight of non-specified component(s).

It will be understood that any component defined herein as being included may be explicitly excluded from the claimed invention by way of proviso or negative limitation. For example, in some aspects the nanocages and/or fusion proteins described herein may exclude a ferritin heavy chain and/or may exclude an iron-binding component.

In addition, all ranges given herein include the end of the ranges and also any intermediate range points, whether explicitly stated or not.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

The abbreviation, “e.g.” is derived from the Latin exempli gratia, and is used herein to indicate a non-limiting example. Thus, the abbreviation “e.g.” is synonymous with the terms “for example,” or “such as.” The word “or” is intended to include “and” unless the context clearly indicates otherwise.

The term “subject” as used herein refers to any member of the animal kingdom, typically a mammal. The term “mammal” refers to any animal classified as a mammal, including humans, other higher primates, domestic and farm animals, and zoo, sports, or pet animals, such as dogs, cats, cattle, horses, sheep, pigs, goats, rabbits, etc. Typically, the mammal is human.

The terms “protein nanoparticle,” “nanocage,” and “Multabody” are used interchangeably herein and refer to a protein-based polyhedron shaped structure made from a plurality of nanocage monomers. These nanocage monomers, or subunits thereof, are each composed of proteins or polypeptides (for example a glycosylated polypeptide), and, optionally of single or multiple features of the following: nucleic acids, prosthetic groups, organic and inorganic compounds. Non-limiting examples of protein nanoparticles include ferritin nanoparticles (see, e.g., Zhang, Y. Int. J. Mol. Sci., 12:5406-5421, 2011, incorporated by reference herein), encapsulin nanoparticles (see, e.g., Sutter et al., Nature Struct, and Mol. Biol., 15:939-947, 2008, incorporated by reference herein), Sulfur Oxygenase Reductase (SOR) nanoparticles (see, e.g., Urich et al., Science, 311:996-1000, 2006, incorporated by reference herein), lumazine synthase nanoparticles (see, e.g., Zhang et al., J. Mol. Biol., 306: 1099-1114, 2001) or pyruvate dehydrogenase nanoparticles (see, e.g., Izard et al., PNAS 96: 1240-1245, 1999, incorporated by reference herein). Ferritin, apoferritin, encapsulin, SOR, lumazine synthase, and pyruvate dehydrogenase are monomeric proteins that self-assemble into a globular protein complexes that in some cases consists of 24, 60, 24, 60, and 60 protein subunits, respectively. Ferritin and apoferritin are generally referred to interchangeably herein and are understood to both be suitable for use in the fusion proteins, nanocages, and methods described herein. Carboxysome, vault proteins, GroEL, heat shock protein, E2P and MS2 coat protein also produce nanocages are contemplated for use herein. In addition, fully or partially synthetic self-assembling monomers are also contemplated for use herein.

It will be understood that each nanocage monomer may be divided into two or more subunits that will self-assemble into a functional nanocage monomer. For example, ferritin or apoferritin may be divided into an N- and C-subunit, e.g., an N- and C-subunit obtained by dividing full-length ferritin substantially in half, so that each subunit may be separately bound to a different scFc fragment or Fc monomer or bioactive moiety (e.g. Fab fragment) for subsequent self-assembly into a nanocage monomer and a nanocage. Each subunit may, in aspects, be linked to an scFc and/or Fc monomer and/or bioactive moiety at both termini, either the same or different. By “functional nanocage monomer or subunit thereof” it is intended that the nanocage monomer or subunit thereof is capable of self-assembly with complementary monomers or subunits into a nanocage as described herein.

The terms “ferritin” and “apoferritin” are used interchangeably herein and generally refer to a polypeptide (e.g., a ferritin chain) that is capable of assembling into a ferritin complex which typically comprises 24 protein subunits. It will be understood that the ferritin can be from any species. Typically, the ferritin is a human ferritin. In some embodiments, the ferritin is a wild-type ferritin. For example, the ferritin may be a wild-type human ferritin. In some embodiments, a ferritin light chain is used as a nanocage monomer, and/or a subunit of a ferritin light chain is used as a nanocage monomer subunit. In some embodiments, assembled nanocages do not include any ferritin heavy chains or other ferritin components capable of binding to iron.

The term “multispecific,” as used herein, refers to the characteristic of having at least two binding sites at which at least two different binding partners, e.g., an antigen or receptor (e.g., Fc receptor), can bind. For example, a nanocage that comprises at least two Fab fragments, wherein each of the two Fab fragments binds to a different antigen, is “multispecific.” As an additional example, a nanocage that comprises an Fc fragment (which is capable of binding to an Fc receptor) and an Fab fragment (which is capable of binding to an antigen) is “multispecific.”

The term “multivalent,” as used herein, refers to the characteristic of having at least two binding sites at which a binding partner, e.g., an antigen or receptor (e.g., Fc receptor), can bind. The binding partners that can bind to the at least two binding sites may be the same or different.

The term “antibody”, also referred to in the art as “immunoglobulin” (Ig), used herein refers to a protein constructed from paired heavy and light polypeptide chains, various Ig isotypes exist, including IgA, IgD, IgE, IgG, such as IgG1, IgG2, IgG3, and IgG4, and IgM. It will be understood that the antibody may be from any species, including human, mouse, rat, monkey, llama, or shark. When an antibody is correctly folded, each chain folds into a number of distinct globular domains joined by more linear polypeptide sequences. For example, in the case of IgGs, the immunoglobulin light chain folds into a variable (V_{L}) and a constant (CL) domain, while the heavy chain folds into a variable (V_{H}) and three constant (C_{H}, C_{H2}, C_{H3}) domains. Interaction of the heavy and light chain variable domains (V_{H} and V_{L}) results in the formation of an antigen binding region (Fv). Each domain has a well-established structure familiar to those of skill in the art.

The light and heavy chain variable regions are responsible for binding the target antigen and can therefore show significant sequence diversity between antibodies. The constant regions show less sequence diversity, and are responsible for binding a number of natural proteins to elicit important immunological events. The variable region of an antibody contains the antigen binding determinants of the molecule, and thus determines the specificity of an antibody for its target antigen. The majority of sequence variability occurs in six hypervariable regions, three each per variable heavy and light chain; the hypervariable regions combine to form the antigen-binding site, and contribute to binding and recognition of an antigenic determinant. The specificity and affinity of an antibody for its antigen is determined by the structure of the hypervariable regions, as well as their size, shape and chemistry of the surface they present to the antigen.

An “antibody fragment” as referred to herein may include any suitable antigen-binding antibody fragment known in the art. The antibody fragment may be a naturally-occurring antibody fragment, or may be obtained by manipulation of a naturally-occurring antibody or by using recombinant methods. For example, an antibody fragment may include, but is not limited to a Fv, single-chain Fv (scFv; a molecule consisting of V_{L} and V_{H} connected with a peptide linker), Fc, single-chain Fc (e.g., a polypeptide comprising two Fc monomers linked together, e.g., via a linker such as an amino acid linker), Fc monomer (e.g., a single Fc chain comprising exactly one CH2 domain and exactly one CH3 domain, which is typically capable of dimerizing with another Fc monomer), Fab, single-chain Fab, F(ab′)₂, single domain antibody (sdAb; a fragment composed of a single V_{L} or V_{H}), and multivalent presentations of any of these. As used herein, “antigen-binding moiety” refers to an antibody or portion of an antibody that specifically binds to a target antigen.

By the term “synthetic antibody” as used herein, is meant an antibody which is generated using recombinant DNA technology. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The phrases “antibody-preventable” and “antibody-treatable” condition, as used herein, generally refer to conditions which are known to be associated with the expression or presence of at least one antigen. For example, in the context of an infectious disease, the antigen may be an antigen on the infectious disease agent, the antigen may be expressed by an infected cell, and/or the antigen may be expressed by a cell involved in the immune response to an infection. “Antibody-preventable conditions” are generally those conditions where an antibody can be used to prevent the condition from becoming established. “Antibody-treatable conditions” are generally those conditions where an antibody can be used to treat and established condition.

The term “epitope” refers to an antigenic determinant. An epitope is the particular chemical groups or peptide sequences on a molecule that are antigenic, that is, that elicit a specific immune response. An antibody specifically binds a particular antigenic epitope, e.g., on a polypeptide. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5, about 9, about 11, or about 8 to about 12 amino acids in a unique spatial conformation. Methods of determining spatial conformation of epitopes include, for example, x-ray crystallography and 2-dimensional nuclear magnetic resonance. See, e.g., “Epitope Mapping Protocols” in Methods in Molecular Biology, Vol. 66, Glenn E. Morris, Ed (1996).

The term “antigen” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequence or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the aspects described herein include, but are not limited to, the use of partial nucleotide sequences of more than one gene and that these nucleotide sequences could be arranged in various combinations to elicit the desired immune response. Moreover, a skilled artisan will understand that an antigen need not be encoded by a “gene” at all. It is readily apparent that an antigen can be synthesized or can be derived from a biological sample. Such a biological sample can include, but is not limited to a tissue sample, a cell, or a biological fluid.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “expression” as used herein is defined as the transcription and/or translation of a particular nucleotide sequence driven by its promoter.

The phrase “HIV-1-related condition,” as used herein, refers to the condition of HIV-1 infection (including primary infection, latent infection) and/or a condition resulting from an HIV-1 infection (e.g., acquired immunodeficiency syndrome).

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, typically, a human.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

“Parenteral” administration of composition includes, e.g., subcutaneous (s.c.), intravenous (i.v.), intramuscular (i.m.), or intrasternal injection, or infusion techniques. Also included are inhalation and intranasal administration.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species, for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

As used herein, “to treat” a condition or “treatment” of the condition (e.g., the conditions described herein such as HIV-1) is an approach for obtaining beneficial or desired results, such as clinical results. Beneficial or desired results can include, but are not limited to, alleviation or amelioration of one or more symptoms or conditions; diminishment of extent of disease, disorder, or condition; stabilized (i.e., not worsening) state of disease, disorder, or condition; preventing spread of disease, disorder, or condition; delay or slowing the progress of the disease, disorder, or condition; amelioration or palliation of the disease, disorder, or condition; and remission (whether partial or total), whether detectable or undetectable. “Palliating” a disease, disorder, or condition means that the extent and/or undesirable clinical manifestations of the disease, disorder, or condition are lessened and/or time course of the progression is slowed or lengthened, as compared to the extent or time course in the absence of treatment. As used herein, the terms “prevention” or “prophylaxis” refers to the reduction in the risk of acquiring or developing a disease or disorder, for example HIV-1, or the reduction or inhibition of the recurrence of a disease or disorder, for example HIV-1. Thus, an HIV-1 therapeutic or prophylactic composition refers to a composition comprising assembled nanocages as described herein, or fusion proteins as described herein that are capable of assembling into nanocages, that when administered to a subject are capable of treating and/or preventing HIV-1.

The terms “therapeutically effective amount”, “effective amount” or “sufficient amount” mean a quantity sufficient, when administered to a subject, including a mammal, for example a human, to achieve a desired result, for example an amount effective to cause a cell death. Effective amounts of the compounds described herein may vary according to factors such as the molecule, age, sex, species, and weight of the subject. Dosage or treatment regimes may be adjusted to provide the optimum therapeutic response, as is understood by a skilled person. For example, administration of a therapeutically effective amount of the fusion proteins described herein is, in aspects, sufficient to treat and/or prevent HIV-1. Moreover, a treatment regime of a subject with a therapeutically effective amount may consist of a single administration, or alternatively comprise a series of applications. The frequency and length of the treatment period depends on a variety of factors, such as the molecule, the age of the subject, the concentration of the agent, the responsiveness of the patient to the agent, or a combination thereof. It will also be appreciated that the effective dosage of the agent used for the treatment may increase or decrease over the course of a particular treatment regime. Changes in dosage may result and become apparent by standard diagnostic assays known in the art. The fusion proteins described herein may, in aspects, be administered before, during or after treatment with conventional therapies for the disease or disorder in question. For example, the fusion proteins described herein may find particular use in combination with conventional treatments for HIV-1.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

Administration “in combination with” one or more further therapeutic agents includes simultaneous (concurrent) and consecutive administration in any order.

The term “pharmaceutically acceptable” means that the compound or combination of compounds is compatible with the remaining ingredients of a formulation for pharmaceutical use, and that it is generally safe for administering to humans according to established governmental standards, including those promulgated by the United States Food and Drug Administration.

The term “pharmaceutically acceptable carrier” includes, but is not limited to solvents, dispersion media, coatings, antibacterial agents, antifungal agents, isotonic and/or absorption delaying agents and the like. The use of pharmaceutically acceptable carriers is well known.

“Variants” are biologically active fusion proteins, antibodies, or fragments thereof having an amino acid sequence that differs from a comparator sequence by virtue of an insertion, deletion, modification and/or substitution of one or more amino acid residues within the comparative sequence. Variants generally have less than 100% sequence identity with the comparative sequence. Ordinarily, however, a biologically active variant will have an amino acid sequence with at least about 70% amino acid sequence identity with the comparative sequence, such as at least about 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity. The variants include peptide fragments of at least 10 amino acids that retain some level of the biological activity of the comparator sequence. Variants also include polypeptides wherein one or more amino acid residues are added at the N- or C-terminus of, or within, the comparative sequence. Variants also include polypeptides where a number of amino acid residues are deleted and/or optionally substituted by one or more amino acid residues. Variants also may be covalently modified, for example by substitution with a moiety other than a naturally occurring amino acid or by modifying an amino acid residue to produce a non-naturally occurring amino acid.

“Percent amino acid sequence identity” is defined herein as the percentage of amino acid residues in the candidate sequence that are identical with the residues in the sequence of interest, such as the polypeptides of the invention, after aligning the sequences and introducing gaps, if necessary, to achieve the maximum percent sequence identity, and not considering any conservative substitutions as part of the sequence identity. None of N-terminal, C-terminal, or internal extensions, deletions or insertions into the candidate sequence shall be construed as affecting sequence identity or homology. Methods and computer programs for the alignment are well known in the art, such as “BLAST”.

“Active” or “activity” for the purposes herein refers to a biological and/or an immunological activity of the fusion proteins described herein, wherein “biological” activity refers to a biological function (either inhibitory or stimulatory) caused by the fusion proteins.

The fusion proteins described herein may include modifications. Such modifications include, but are not limited to, conjugation to an effector molecule. Modifications further include, but are not limited to conjugation to detectable reporter moieties. Modifications that extend half-life (e.g., pegylation) are also included. Modifications for de-immunization are also included. Proteins and non-protein agents may be conjugated to the fusion proteins by methods that are known in the art. Conjugation methods include direct linkage, linkage via covalently attached linkers, and specific binding pair members (e.g., avidin-biotin). Such methods include, for example, that described by Greenfield et al., Cancer Research 50, 6600-6607 (1990), which is incorporated by reference herein and those described by Amon et al., Adv. Exp. Med. Biol. 303, 79-90 (1991) and by Kiseleva et al, Mol. Biol. (USSR)25, 508-514 (1991), both of which are incorporated by reference herein.

### Fusion Proteins Comprising an Fc Monomer

Described herein are fusion proteins. In aspects, the fusion proteins comprise a nanocage monomer or subunit thereof linked to an Fc monomer, wherein a plurality of the fusion proteins self-assemble to form a nanocage comprising one or more Fc dimers. In this way, the assembled one or more Fc dimers may decorate the interior surface of the assembled nanocage, the exterior surface of the assembled nanocage, or both. In some embodiments, the assembled one or more Fc dimers decorate the exterior surface of the assembled nanocage. By dividing an Fc dimer into monomeric components, additional control is afforded in nanocage design. The Fc monomers will assist in assembling the nanocage and into a tighter control of the ratios of other linked components, such as bioactive moieties.

In some aspects, a nanocage monomer subunit is used in the fusion protein instead of a full nanocage monomer. The nanocage monomer can be divided into two portions, one of which comprises the N-terminal end of the nanocage monomer, referred to as the “N-subunit,” and the other of which comprises the C-terminal end of the nanocage monomer, referred to as the “C-subunit.” The N- or C-subunits may each represent substantially one half of the nanocage monomer, or they may be unevenly divided. Typically, the N-subunit and the C-subunit correspond substantially to the N-terminal half of the nanocage monomer and the C-terminal half of the nanocage monomer, respectively. The N-subunit and the C-subunit are capable of self-assembling to form the nanocage monomer, which is itself capable of self-assembling to form the nanocage as described above.

It will be understood that each nanocage monomer and each N- and C-subunit comprises an N-terminus and a C-terminus. An Fc monomer can be linked to the nanocage monomer or subunit thereof at the either or both termini. Typically, the Fc monomer is linked to the nanocage monomer or subunit thereof at the C-terminus. Likewise, the Fc monomer can be linked to either or both termini of the N-subunit and/or C-subunit. Typically, the Fc monomer is linked to the C-subunit at the C-terminus.

In typical aspects, the nanocage monomer or subunit thereof is further linked to a bioactive moiety. Like with the Fc monomer, the bioactive moiety can be linked to the nanocage monomer or subunit thereof at the N- or C-terminus of the nanocage monomer or subunit thereof and is typically linked at the N-terminus. When a nanocage monomer subunit is used, the bioactive moiety can likewise be linked to the N-subunit or C-subunit at either terminus, typically at the opposite terminus from the Fc monomer. Typically, the bioactive moiety is linked to the C-subunit at the N-terminus.

The antigen-binding moiety is linked to the nanocage monomer or subunit thereof so that it decorates the interior and/or exterior surface of the assembled nanocage, typically it decorates the exterior surface of the assembled nanocage.

The bioactive moiety is typically an antibody or a fragment thereof that specifically binds to an antigenic target. It will be understood that the antibody or fragment thereof may comprise or consist of, for example, a heavy and/or light chain of a Fab fragment. The antibody or fragment thereof may comprise or consist of a Fab (e.g., scFab) fragment, a scFv fragment, a sdAb fragment, and/or a VHH region for example. In some embodiments, the bioactive moiety does not comprise any CH2 or CH3 domains. It will be understood that any antibody or fragment thereof may be used in the fusion proteins described herein.

Generally, the fusion protein described herein is associated with a Fab light chain and/or heavy chain, which may be produced separately or contiguously with the fusion protein.

In typical aspects, the antigen-binding moiety binds specifically to an antigen associated with an antibody-preventable and/or antibody-treatable condition. For example, the antigen may be associated with an infectious agent, including a virus, bacteria, a parasite, a fungus, or a yeast, a cancer, or an immune disease, including an autoimmune disease.

In exemplary aspects, the antigen-binding moiety comprises an HIV-1 specific antigen-binding moiety. The HIV-1-specific antigen-binding moiety may bind to BG505 SOSIP_D368R, 93TH057 gp120, and/or an MPER peptide, for example. In specific examples, the HIV-1 specific antigen-binding moiety comprises an antigen-binding moiety from PGDM1400, 10E8v4, and/or N49P7.

In certain aspects, the nanocage monomer described herein comprises an N-subunit or C-subunit linked to Fc monomer or to a bioactive motive (e.g. Fab fragment). The N- or C-subunit is capable of self-assembling with a complementary C- or N-subunit to form a full nanocage monomer, a plurality of which self-assemble to form the nanocage, thus allowing for multiple Fc monomers and/or other moieties to self assemble into one nanocage. Amounts of each different component are controlled by controlling gene and expression ratios. These nanocage monomer subunits can be used alone or in combination.

For example, the Fc monomer or the bioactive moiety (e.g. the Fab fragment) can be linked to a divided apoferritin monomer (N- or C-subunit, which are each typically about half of a full-length apoferritin monomer). Each subunit fused to the Fc monomer or the bioactive moiety (e.g. Fab fragment) self-assembles into an apoferritin monomer that in turn self-assembles with other apoferritin monomers (either a full apoferritin or an assembled apoferritin formed of N- and C-subunits) to form a nanocage.

When a full-length nanocage monomer is used, the nanocage monomer typically comprises an Fc monomer linked at the C-terminus of the nanocage monomer and a bioactive moiety linked at the N-terminus of the nanocage monomer. When a nanocage monomer subunit is used, the subunit typically comprises an Fc monomer linked at the C-terminus of the subunit and a bioactive moiety linked at the N-terminus of the subunit. Typically, the C-subunit is used in the fusion proteins described herein, with the Fc monomer linked at the C-terminus of the C-subunit and the bioactive moiety linked at the N-terminus of the C-subunit.

The N- or C-subunit described herein is, in aspects, provided in combination with the complementary C- or N-subunit, with which the N- or C-subunit is capable of self-assembling. The complementary C- or N-subunit may or may not be a fusion protein. In some aspects, the complementary C- or N-subunit is linked to a bioactive moiety at the N- or C-terminus, typically the N-terminus. The bioactive moiety may be, for example, an antigen-binding moiety such as an Fab fragment, which may be the same or different from any bioactive moiety or antigen-binding moiety linked to the N- or C-subunit. The complementary C- or N-subunit may be linked to an Fc monomer at the N- or C-terminus thereof, typically the C-terminus. It will be understood that, typically, the Fc monomer is fused to the C-terminus of the C-subunit of ferritin in order to take advantage of the 4-fold axes of the nanocage and thereby meeting the other half.

For example, in some aspects, the fusion protein comprises a C-subunit linked to an Fc monomer at the C-terminus and an Fab fragment at the N-terminus. In use, the C-subunit self-assembles with an N-subunit linked to an Fab fragment at its N-terminus, which may be the same or different from the Fab fragment linked to the C-subunit, to form the nanocage monomer. As described above, a plurality of the nanocage monomers self-assemble to form a nanocage. The nanocage monomer subunits may be provided alone or in combination and may have the same or different bioactive moieties fused thereto.

The Fc monomer may be any Fc monomer derived from any antibody type or species. Typically, the Fc monomer is human and is derived from an IgG, IgA, IgD, IgM, or IgE. For example, the Fc monomer may be derived from an IgG, such as IgG1, IgG2, IgG3, or IgG4, such as IgG1 Fc monomer.

The Fc monomer may comprise one or more mutations or sets of mutations that modulate the half-life of the fusion protein from, for example, minutes or hours to several days, weeks, or months. For example, the Fc monomer may comprise a mutation at one or more of L234, L235, G236, G237, M252, I253, S254, T256, P329, A330, M428, N434, or a combination thereof (wherein numbering is according to the EU index), such as M428L and N434S (“LS”); M252Y, S254T and T256E (“YTE”); L234A and L235A (“LALA”); I253A; L234A, L235A, and P329G (“LALAP”); G236R; G237A; and/or A330L or a combination thereof.

Moreover, other substitutions in the fusion proteins and nanocages described herein are contemplated, including Fc sequence modifications and addition of other agents (e.g. human serum albumin, human serum albumin peptide sequences and antibodies such as Fabs and/or nanobodies targeting human serum albumin), that allow changes in bioavailability and will be understood by a skilled person. Furthermore, the fusion proteins and nanocages described herein can be modulated in sequence or by addition of other agents to mute immunogenicity and anti-drug responses (therapeutic, e.g. matching sequence to host, or addition of immunosuppressive therapies [such as, for example, methotrexate when administering infliximab for treating rheumatoid arthritis or induction of neonatal tolerance, which is a primary strategy in reducing the incidence of inhibitors against FVIII (reviewed in: DiMichele D M, Hoots W K, Pipe S W, Rivard G E, Santagostino E. International workshop on immune tolerance induction: consensus recommendations. Haemophilia. 2007; 13:1-22, incorporated herein by reference in its entirety]).

As described herein, the nanocage monomer may be formed from subunits. Thus the nanocage monomer subunit may comprise a first apoferritin subunit, optionally a first human apoferritin subunit, such as an apoferritin N- or C-subunit, and wherein the first apoferritin subunit is capable of self-assembling with a second apoferritin subunit, such as a complementary C- or N-subunit. It will be understood that other nanocage monomers can be divided into bipartite subunits much like apoferritin as described herein so that the subunits self-assemble and are each amenable to fusion with a bioactive moiety.

The “N” region of apoferritin typically comprises or consists of a sequence at least 70% (such as at least 75%, 80%, 85%, 90%, 95%, 96%, 97%, 98%, 99%, or 100%) identical to:

(SEQ ID NO: 1)
MSSQIRQNYSTDVEAAVNSLVNLYLQASYTYLSLGFYFDRDDVALEGVS
HFFRELAEEKREGYERLLKMQNQRGGRALFQDIKKPAEDEW.

It will be understood that anywhere herein where the “N” region of apoferritin is described, alone or as a component of another fusion protein, in full or in part, the sequence MSSQIRQNYSTDVEAAVNSLVNLYLQASYTYLSLGFYFDRDDVALEGVSHFFRELAEEKREGYERLL KMQNQRGGRALFQDIKKPAEDEW (SEQ ID NO:1) may be replaced with SSQIRQNYSTDVEAAVNSLVNLYLQASYTYLSLGFYFDRDDVALEGVSHFFRELAEEKREGYERLLK MQNQRGGRALFQDIKKPAEDEW (SEQ ID NO:15). Thus, in aspects, the “N” region of apoferritin typically comprises or consists of a sequence at least 70% (such as at least 75%, 80%, 85%, 90%, 95%, 96%, 97%, 98%, 99%, or 100%) identical to:

(SEQ ID NO: 15)
SSQIRQNYSTDVEAAVNSLVNLYLQASYTYLSLGFYFDRDDVALEGVSH
FFRELAEEKREGYERLLKMQNQRGGRALFQDIKKPAEDEW

The “C” region of apoferritin typically comprises or consists of a sequence at least 70% (such as at least 75%, 80%, 85%, 90%, 95%, 96%, 97%, 98%, 99%, or 100%) identical to:

(SEQ ID NO: 2)
GKTPDAMKAAMALEKKLNQALLDLHALGSARTDPHLCDFLETHFLDEEV
KLIKKMGDHLTNLHRLGGPEAGLGEYLFERLTLRHD.

It will be understood that anywhere herein where the “C” region of apoferritin is described, alone or as a component of another fusion protein, in full or in part, the sequence GKTPDAMKAAMALEKKLNQALLDLHALGSARTDPHLCDFLETHFLDEEVKLIKKMGDHLTNLHRLGG PEAGLGEYLFERLTLRHD (SEQ ID NO:2) may be replaced with GKTPDAMKAAMALEKKLNQALLDLHALGSARTDPHLCDFLETHFLDEEVKLIKKMGDHLTNLHRLGG PEAGLGEYLFERLTLKHD (SEQ ID NO: 16). Thus, in aspects, the “C” region of apoferritin typically comprises or consists of a sequence at least 70% (such as at least 75%, 80%, 85%, 90%, 95%, 96%, 97%, 98%, 99%, or 100%) identical to:

(SEQ ID NO: 16)
GKTPDAMKAAMALEKKLNQALLDLHALGSARTDPHLCDFLETHFLDEEV
KLIKKMGDHLTNLHRLGGPEAGLGEYLFERLTLKHD

In cases where the antibody or fragment thereof comprises two chains, such as a first and second chain, or a heavy and light chain, the two chains are optionally separated by a linker. The linker may be flexible or rigid, but it typically is flexible to allow the chains to fold appropriately. Similarly, the Fc monomer and/or the bioactive moiety is typically linked to the nanocage monomer or subunit thereof through a linker.

The linker is generally long enough to impart some flexibility to the fusion protein, although it will be understood that linker length will vary depending upon the nanocage monomer or subunit thereof and Fc monomer and bioactive moiety sequences and the three-dimensional conformation of the fusion protein. Thus, the linker is typically from about 1 to about 130 amino acid residues, such as from about 1, 5, 10, 15, 20, 25, 30, 35, 40, 45, 50, 55, 60, 65, 70, 75, 80, 85, 90, 95, 100, 105, 110, 115, 120, or 125 to about 5, 10, 15, 20, 25, 30, 35, 40, 45, 50, 55, 60, 65, 70, 75, 80, 85, 90, 95, 100, 105, 110, 115, 120, 125, or 130 amino acid residues, or from about 50 to about 90 amino acid residues, such as 70 amino acid residues, or from about 1 to about 100 amino acid residues, such as from about 1 to about 70 amino acid residues, such as from about 1 to about 30 amino acid residues, such as from about 8 to about 16 amino acid residues.

The linker may be of any amino acid sequence and, in one typical example, the linker comprises a GS domain, or a series of G and S amino acids, such as a series of GS repeats, GGS repeats, GGGS repeats, and/or GGGGS repeats. Typically, the linker comprises a GGGGS and/or GGGS repeat and, more typically, the linker comprises at least about 2, 3, 4, 5, 6, 7, 8, 9, 10, 11, 12, 13, 14, 15, 16, 17, 18, 19, or 20 GGGS and/or GGGGS repeats, such as about 5 GGGS repeats and/or about 14 GGGGS repeats. In specific aspects, the linker comprises or consists of a sequence at least 70% (such as at least 75%, 80%, 85%, 90%, 95%, 96%, 97%, 98%, 99%, or 100%) identical to:

(SEQ ID NO: 3)
GGGGSGGGGSGGGGSGGGGSGGGGSGGGGSGGGGSGGGGSGGGGSGGGG
SGGGGSGGGGSGGGGSGGGGS,
(SEQ ID NO: 4)
GGGGSGGGGSGGGGSGGGGSGGGGSGG,
or
(SEQ ID NO: 5)
GGSGGSGGSGGSGGGSGGSGGSGGSG.

In typical aspects, the antibody or fragment thereof binds specifically to an antigen associated with HIV-1.

In aspects, the fusion protein described herein comprises or consists of sequences at least 70% (such as at least 75%, 80%, 85%, 90%, 95%, 96%, 97%, 98%, 99%, or 100%) identical to one or more of the following sequences, wherein the ferritin subunit is in bold and linkers are underlined. While in each of these cases either the full ferritin monomer is shown (MSSQIRQNYSTDVEAAVNSLVNLYLQASYTYLSLGFYFDRDDVALEGVSHFFRELAEEKREGYERLL KMQNQRGGRALFQDIKKPAEDEWGKTPDAMKAAMALEKKLNQALLDLHALGSARTDPHLCDFLETH FLDEEVKLIKKMGDHLTNLHRLGGPEAGLGEYLFERLTLRHD; (SEQ ID NO:6), or the N- or C-ferritin monomer is shown (MSSQIRQNYSTDVEAAVNSLVNLYLQASYTYLSLGFYFDR DDVALEGVSHFFRELAEEKREGYERLLKMQNQRGGRALFQDIKKPAEDEW (SEQ ID NO:1) in the case of N-ferritin and GKTPDAMKAAMALEKKLNQALLDLHALGSARTDPHLCDFLETHFLDEEVKLIK KMGDHLTNLHRLGGPEAGLGEYLFERLTLRHD (SEQ ID NO:2) in the case of C-ferritin), it will be understood that these could be interchangeably used or another monomer or part thereof could be used in its place.

### 32-N MB.v3:

N49P7_N-Ferritin
(SEQ ID NO: 7)
QSALTQPRSVSASPGQSVTISCTGTHNLVSWCQHQPGRAPKLLIYDFNKRPSGVPDRFSGS
GSGGTASLTITGLQDDDDAEYFCWAYEAFGGGTKLTVLGQPKAAPSVTLFPPSSEELQANKATLVCLI
SDFYPGAVTVAWKADSSPVKAGVETTTPSKQSNNKYAASSYLSLTPEQWKSHRSYSCQVTHEGSTV
EKTVAPTECGGGGSGGGGSGGGGSGGGGSGGGGSGGGGSGGGGSGGGGSGGGGSGGGGSG
GGGSGGGGSGGGGSGGGGSADLVQSGAVVKKPGDSVRISCEAQGYRFPDYIIHWIRRAPGQGPE
WMGWMNPMGGQVNIPWKFQGRVSMTRDTSIETAFLDLRGLKSDDTAVYYCVRDRSNGSGKRFES
SNWFLDLWGRGTAVTIQSASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDKKVEPKSCGGGGSGGGGSG
GGGSGGGGSGGGGSGGMSSQIRQNYSTDVEAAVNSLVNLYLQASYTYLSLGFYFDRDDVALEGV
SHFFRELAEEKREGYERLLKMQNQRGGRALFQDIKKPAEDEW
PGDM1400_hFerritin
(SEQ ID NO: 8)
DFVLTQSPHSLSVTPGESASISCKSSHSLIHGDRNNYLAWYVQKPGRSPQLLIYLASSRASG
VPDRFSGSGSDKDFTLKISRVETEDVGTYYCMQGRESPWTFGQGTKVDIKRTVAAPSVFIFPPSDEQ
LKSGTASVVCLLNNFYPREAKVQWKVDNALQSGNSQESVTEQDSKDSTYSLSSTLTLSKADYEKHKV
YACEVTHQGLSSPVTKSFNRGECGGGGSGGGGSGGGGSGGGGSGGGGSGGGGSGGGGSGGG
GSGGGGSGGGGSGGGGSGGGGSGGGGSGGGGSQAQLVQSGPEVRKPGTSVKVSCKAPGNTLK
TYDLHWVRSVPGQGLQWMGWISHEGDKKVIVERFKAKVTIDWDRSTNTAYLQLSGLTSGDTAVYYC
AKGSKHRLRDYALYDDDGALNWAVDVDYLSNLEFWGQGTAVTVSSASTKGPSVFPLAPSSKSTSGG
TAALGCLVKDYFPEPVTVSWNSGALTSGVHTFPAVLQSSGLYSLSSVVTVPSSSLGTQTYICNVNHK
PSNTKVDKKVEPKSCDGGGGSGGGGSGGGGSGGGGSGGGGSGGMSSQIRQNYSTDVEAAVNSL
VNLYLQASYTYLSLGFYFDRDDVALEGVSHFFRELAEEKREGYERLLKMQNQRGGRALFQDIKKP
AEDEWGKTPDAMKAAMALEKKLNQALLDLHALGSARTDPHLCDFLETHFLDEEVKLIKKMGDHLT
NLHRLGGPEAGLGEYLFERLTLRHD
or
(SEQ ID NO: 17)
DFVLTQSPHSLSVTPGESASISCKSSHSLIHGDRNNYLAWYVQKPGRSPQLLIYLASSRASG
VPDRFSGSGSDKDFTLKISRVETEDVGTYYCMQGRESPWTFGQGTKVDIKRTVAAPSVFIFPPSDEQ
LKSGTASVVCLLNNFYPREAKVQWKVDNALQSGNSQESVTEQDSKDSTYSLSSTLTLSKADYEKHKV
YACEVTHQGLSSPVTKSFNRGECGGGGSGGGGSGGGGSGGGGSGGGGSGGGGSGGGGSGGG
GSGGGGSGGGGSGGGGSGGGGSGGGGSGGGGSQAQLVQSGPEVRKPGTSVKVSCKAPGNTLK
TYDLHWVRSVPGQGLQWMGWISHEGDKKVIVERFKAKVTIDWDRSTNTAYLQLSGLTSGDTAVYYC
AKGSKHRLRDYALYDDDGALNWAVDVDYLSNLEFWGQGTAVTVSSASTKGPSVFPLAPSSKSTSGG
TAALGCLVKDYFPEPVTVSWNSGALTSGVHTFPAVLQSSGLYSLSSVVTVPSSSLGTQTYICNVNHK
PSNTKVDKKVEPKSCGGGGSGGGGSGGGGSGGGGSGGGGSGGSSQIRQNYSTDVEAAVNSLVN
LYLQASYTYLSLGFYFDRDDVALEGVSHFFRELAEEKREGYERLLKMQNQRGGRALFQDIKKPAE
DEWGKTPDAMKAAMALEKKLNQALLDLHALGSARTDPHLCDFLETHFLDEEVKLIKKMGDHLTNL
HRLGGPEAGLGEYLFERLTLKHD
10E8v4_C-Ferritin_Fc1
(SEQ ID NO: 9)
SELTQDPAVSVALKQTVTITCRGDSLRSHYASWYQKKPGQAPVLLFYGKNNRPSGIPDRFS
GSASGNRASLTITGAQAEDEADYYCSSRDKSGSRLSVFGGGTKLTVLSQPKAAPSVTLFPPSSEELQ
ANKATLVCLISDFYPGAVTVAWKADSSPVKAGVETTTPSKQSNNKYAASSYLSLTPEQWKSHRSYSC
QVTHEGSTVEKTVAPTECGGGGSGGGGSGGGGSGGGGSGGGGSGGGGSGGGGSGGGGSGGG
GSGGGGSGGGGSGGGGSGGGGSGGGGSEVRLVESGGGLVKPGGSLRLSCSASGFDFDNAWMT
WVRQPPGKGLEWVGRITGPGEGWSVDYAESVKGRFTISRDNTKNTLYLEMNNVRTEDTGYYFCAR
TGKYYDFWSGYPPGEEYFQDWGQGTLVIVSSASTKGPSVFPLAPSSKSTSGGTAALGCLVKDYFPE
PVTVSWNSGALTSGVHTFPAVLQSSGLYSLSSVVTVPSSSLGTQTYICNVNHKPSNTKVDKKVEPKS
CGGGGSGGGGSGGGGSGGGGSGGGGSGGKTPDAMKAAMALEKKLNQALLDLHALGSARTDPH
LCDFLETHFLDEEVKLIKKMGDHLTNLHRLGGPEAGLGEYLFERLTLRHDGGGGSGGGGSGGGGS
GGGGSGGGGSGDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPVLDSDGSFFLYSK
LTVDKSRWQQGNVFSCSVMHEALHNHYTQKSLSLSPGK
32-N MB.v3 LS:
N49P7_N-Ferritin
(SEQ ID NO: 7)
QSALTQPRSVSASPGQSVTISCTGTHNLVSWCQHQPGRAPKLLIYDFNKRPSGVPDRFSGS
GSGGTASLTITGLQDDDDAEYFCWAYEAFGGGTKLTVLGQPKAAPSVTLFPPSSEELQANKATLVCLI
SDFYPGAVTVAWKADSSPVKAGVETTTPSKQSNNKYAASSYLSLTPEQWKSHRSYSCQVTHEGSTV
EKTVAPTECGGGGSGGGGSGGGGSGGGGSGGGGSGGGGSGGGGSGGGGSGGGGSGGGGSG
GGGSGGGGSGGGGSGGGGSADLVQSGAVVKKPGDSVRISCEAQGYRFPDYIIHWIRRAPGQGPE
WMGWMNPMGGQVNIPWKFQGRVSMTRDTSIETAFLDLRGLKSDDTAVYYCVRDRSNGSGKRFES
SNWFLDLWGRGTAVTIQSASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDKKVEPKSCGGGGSGGGGSG
GGGSGGGGSGGGGSGGMSSQIRQNYSTDVEAAVNSLVNLYLQASYTYLSLGFYFDRDDVALEGV
SHFFRELAEEKREGYERLLKMQNQRGGRALFQDIKKPAEDEW
PGDM1400_hFerritin
(SEQ ID NO: 8)
DFVLTQSPHSLSVTPGESASISCKSSHSLIHGDRNNYLAWYVQKPGRSPQLLIYLASSRASG
VPDRFSGSGSDKDFTLKISRVETEDVGTYYCMQGRESPWTFGQGTKVDIKRTVAAPSVFIFPPSDEQ
LKSGTASVVCLLNNFYPREAKVQWKVDNALQSGNSQESVTEQDSKDSTYSLSSTLTLSKADYEKHKV
YACEVTHQGLSSPVTKSFNRGECGGGGSGGGGSGGGGSGGGGSGGGGSGGGGSGGGGSGGG
GSGGGGSGGGGSGGGGSGGGGSGGGGSGGGGSQAQLVQSGPEVRKPGTSVKVSCKAPGNTLK
TYDLHWVRSVPGQGLQWMGWISHEGDKKVIVERFKAKVTIDWDRSTNTAYLQLSGLTSGDTAVYYC
AKGSKHRLRDYALYDDDGALNWAVDVDYLSNLEFWGQGTAVTVSSASTKGPSVFPLAPSSKSTSGG
TAALGCLVKDYFPEPVTVSWNSGALTSGVHTFPAVLQSSGLYSLSSVVTVPSSSLGTQTYICNVNHK
PSNTKVDKKVEPKSCDGGGGSGGGGSGGGGSGGGGSGGGGSGGMSSQIRQNYSTDVEAAVNSL
VNLYLQASYTYLSLGFYFDRDDVALEGVSHFFRELAEEKREGYERLLKMQNQRGGRALFQDIKKP
AEDEWGKTPDAMKAAMALEKKLNQALLDLHALGSARTDPHLCDFLETHFLDEEVKLIKKMGDHLT
NLHRLGGPEAGLGEYLFERLTLRHD
10E8v4_C-Ferritin_Fc1-LS
(SEQ ID NO: 10)
SELTQDPAVSVALKQTVTITCRGDSLRSHYASWYQKKPGQAPVLLFYGKNNRPSGIPDRFS
GSASGNRASLTITGAQAEDEADYYCSSRDKSGSRLSVFGGGTKLTVLSQPKAAPSVTLFPPSSEELQ
ANKATLVCLISDFYPGAVTVAWKADSSPVKAGVETTTPSKQSNNKYAASSYLSLTPEQWKSHRSYSC
QVTHEGSTVEKTVAPTECGGGGSGGGGSGGGGSGGGGSGGGGSGGGGSGGGGSGGGGSGGG
GSGGGGSGGGGSGGGGSGGGGSGGGGSEVRLVESGGGLVKPGGSLRLSCSASGFDFDNAWMT
WVRQPPGKGLEWVGRITGPGEGWSVDYAESVKGRFTISRDNTKNTLYLEMNNVRTEDTGYYFCAR
TGKYYDFWSGYPPGEEYFQDWGQGTLVIVSSASTKGPSVFPLAPSSKSTSGGTAALGCLVKDYFPE
PVTVSWNSGALTSGVHTFPAVLQSSGLYSLSSVVTVPSSSLGTQTYICNVNHKPSNTKVDKKVEPKS
CGGGGSGGGGSGGGGSGGGGSGGGGSGGGGKTPDAMKAAMALEKKLNQALLDLHALGSARTD
PHLCDFLETHFLDEEVKLIKKMGDHLTNLHRLGGPEAGLGEYLFERLTLRHDGGSGGSGGSGGSG
GGSGGSGGSGGSGDKTHTCPPCPAPELLGGPSVFLFPPKPKDTLMISRTPEVTCVVVDVSHEDPEV
KFNWYVDGVEVHNAKTKPREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENNYKTTPPVLDSDGSFFL
YSKLTVDKSRWQQGNVFSCSVLHEALHSHYTQKSLSLSPGK;

or combinations thereof.

In further aspects, the fusion protein is conjugated to or associated with a further moiety, such as a detectable moiety (e.g., a small molecule, fluorescent molecule, radioisotope, or magnetic particle), a pharmaceutical agent, a diagnostic agent, or combinations thereof and may comprise, for example, an antibody-drug conjugate.

In aspects wherein the further moiety is a detectable moiety, the detectable moiety may comprise a fluorescent protein, such as GFP, EGFP, Ametrine, and/or a flavin-based fluorescent protein, such as a LOV-protein, such as iLOV.

In aspects wherein the further moiety is a pharmaceutical agent, the pharmaceutical agent may comprise for example, a small molecule, peptide, lipid, carbohydrate, or toxin.

In typical aspects, the nanocage assembled from the fusion proteins described herein comprises from about 3 to about 100 nanocage monomers, none, some, or all of which may be provided as bipartite nanocage monomer subunits, such as from about 3, 4, 5, 6, 7, 8, 9, 10, 12, 14, 16, 18, 20, 22, 24, 26, 28, 30, 32, 34, 36, 38, 40, 42, 44, 46, 48, 50, 52, 55, 56, 58, 60, 62, 64, 66, 68, 70, 72, 74, 76, 78, 80, 82, 84, 86, 88, 90, 92, 94, 96, or 98 to about 4, 5, 6, 7, 8, 9, 10, 12, 14, 16, 18, 20, 22, 24, 26, 28, 30, 32, 34, 36, 38, 40, 42, 44, 46, 48, 50, 52, 55, 56, 58, 60, 62, 64, 66, 68, 70, 72, 74, 76, 78, 80, 82, 84, 86, 88, 90, 92, 94, 96, 98, or 100 nanocage monomers, such as 24, 32, or 60 nanocage monomers. The nanocage monomer or subunit thereof may be any known nanocage monomer, natural, synthetic, or partly synthetic and is, in aspects, selected from ferritin, apoferritin, encapsulin, SOR, lumazine synthase, pyruvate dehydrogenase, carboxysome, vault proteins, GroEL, heat shock protein, E2P, MS2 coat protein, fragments thereof, and variants thereof. Typically, the nanocage monomer or subunit thereof is ferritin or apoferritin or a subunit thereof.

Also described herein are pairs of the fusion proteins described above, wherein each pair self-assembles to form a nanocage monomer, wherein the first and second nanocage monomer subunits are fused to different bioactive moieties as described herein. This provides multivalency and/or multispecificity to a single nanocage monomer assembled from the pair of subunits.

Fusion Proteins Comprising a C-Terminal scFc Fragment

Described herein are fusion proteins. In aspects, the fusion proteins comprise a nanocage monomer or subunit thereof linked to an scFc fragment at the C-terminus of the nanocage monomer or subunit thereof, wherein a plurality of the fusion proteins self-assemble to form a nanocage. In this way, the scFc fragments may decorate the interior surface of the assembled nanocage, the exterior surface of the assembled nanocage, or both. In some embodiments, the scFc fragments decorate the exterior surface of the assembled nanocage. By placing the scFc fragment at the C-terminal end of the nanocage monomer or subunit thereof, surprisingly improved potency and neutralization efficiency was observed.

In some aspects, a nanocage monomer subunit is used in the fusion protein instead of a full nanocage monomer. The nanocage monomer can be divided into two portions, one of which comprises the N-terminal end of the nanocage monomer, referred to as the “N-subunit,” and the other of which comprises the C-terminal end of the nanocage monomer, referred to as the “C-subunit.” The N- or C-subunits may each represent substantially one half of the nanocage monomer, or they may be unevenly divided. Typically, the N-subunit and the C-subunit correspond substantially to the N-terminal half of the nanocage monomer and the C-terminal half of the nanocage monomer, respectively. The N-subunit and the C-subunit are capable of self-assembling to form the nanocage monomer, which is itself capable of self-assembling to form the nanocage as described above.

It will be understood that each nanocage monomer and each N- and C-subunit comprises an N-terminus and a C-terminus. An scFc fragment can be linked to the nanocage monomer or subunit thereof at the either or both termini. Typically, the scFc fragment is linked to the nanocage monomer or subunit thereof at the C-terminus. Likewise, the scFc fragment can be linked to either or both termini of the N-subunit and/or C-subunit. Typically, the scFc fragment is linked to the C-subunit at the C-terminus.

In typical aspects, the nanocage monomer or subunit thereof is further linked to a bioactive moiety. Like with the scFc fragment, the bioactive moiety can be linked to the nanocage monomer or subunit thereof at the N- or C-terminus of the nanocage monomer or subunit thereof and is typically linked at the N-terminus. When a nanocage monomer subunit is used, the bioactive moiety can likewise be linked to the N-subunit or C-subunit at either terminus, typically at the opposite terminus from the scFc fragment. Typically, the bioactive moiety is linked to the C-subunit at the N-terminus.

The antigen-binding moiety is linked to the nanocage monomer or subunit thereof so that it decorates the interior and/or exterior surface of the assembled nanocage, typically it decorates the exterior surface of the assembled nanocage.

The bioactive moiety is typically an antibody or a fragment thereof that specifically binds to an antigenic target. It will be understood that the antibody or fragment thereof may comprise or consist of, for example, a heavy and/or light chain of a Fab fragment. The antibody or fragment thereof may comprise or consist of a Fab (e.g., scFab) fragment, a scFv fragment, a sdAb fragment, and/or a VHH region for example. In some embodiments, the bioactive moiety does not comprise any CH2 or CH3 domains. It will be understood that any antibody or fragment thereof may be used in the fusion proteins described herein.

Generally, the fusion protein described herein is associated with a Fab light chain and/or heavy chain, which may be produced separately or contiguously with the fusion protein.

In typical aspects, the antigen-binding moiety binds specifically to an antigen associated with an antibody-preventable and/or antibody-treatable condition. For example, the antigen may be associated with an infectious agent, including a virus, bacteria, a parasite, a fungus, or a yeast, a cancer, or an immune disease, including an autoimmune disease.

In exemplary aspects, the antigen-binding moiety comprises an HIV-1 specific antigen-binding moiety. The HIV-1-specific antigen-binding moiety may bind to BG505 SOSIP_D368R, 93TH057 gp120, and/or an MPER peptide, for example. In specific examples, the HIV-1 specific antigen-binding moiety comprises an antigen-binding moiety from PGDM1400, 10E8v4, and/or N49P7.

In certain aspects, the nanocage monomer described herein comprises an N-subunit or C-subunit linked to the scFc fragment or to a bioactive motive (e.g. Fab fragment). The N- or C-subunit is capable of self-assembling with a complementary C- or N-subunit to form a full nanocage monomer, a plurality of which self-assemble to form the nanocage, thus allowing for multiple scFc fragments and/or other moieties to self assemble into one nanocage. Amounts of each different component are controlled by controlling gene and expression ratios. These nanocage monomer subunits can be used alone or in combination.

For example, the scFc fragment or the bioactive moiety (e.g. the Fab fragment) can be linked to a divided apoferritin monomer (N- or C-subunit, which are each typically about half of a full-length apoferritin monomer). Each subunit fused to the scFc fragment or the bioactive moiety (e.g. Fab fragment) self-assembles into an apoferritin monomer that in turn self-assembles with other apoferritin monomers (either a full apoferritin or an assembled apoferritin formed of N- and C-subunits) to form a nanocage.

When a full-length nanocage monomer is used, the nanocage monomer typically comprises an scFc fragment linked at the C-terminus of the nanocage monomer and a bioactive moiety linked at the N-terminus of the nanocage monomer. When a nanocage monomer subunit is used, the subunit typically comprises an scFc fragment linked at the C-terminus of the subunit and a bioactive moiety linked at the N-terminus of the subunit. Typically, the C-subunit is used in the fusion proteins described herein, with the scFc fragment linked at the C-terminus of the C-subunit and the bioactive moiety linked at the N-terminus of the C-subunit.

The N- or C-subunit described herein is, in aspects, provided in combination with the complementary C- or N-subunit, with which the N- or C-subunit is capable of self-assembling. The complementary C- or N-subunit may or may not be a fusion protein. In some aspects, the complementary C- or N-subunit is linked to a bioactive moiety at the N- or C-terminus, typically the N-terminus. The bioactive moiety may be, for example, an antigen-binding moiety such as an Fab fragment, which may be the same or different from any bioactive moiety or antigen-binding moiety moiety linked to the N- or C-subunit. The complementary C- or N-subunit may be linked to an scFc fragment at the N- or C-terminus thereof, typically the C-terminus

For example, in some aspects, the fusion protein comprises a C-subunit linked to an scFc fragment at the C-terminus and an Fab fragment at the N-terminus. In use, the C-subunit self-assembles with an N-subunit linked to an Fab fragment at its N-terminus, which may be the same or different from the Fab fragment linked to the C-subunit, to form the nanocage monomer. As described above, a plurality of the nanocage monomers self-assemble to form a nanocage. The nanocage monomer subunits may be provided alone or in combination and may have the same or different bioactive moieties fused thereto.

The scFc fragment may be any scFc fragment derived from any antibody type or species. Typically, the scFc fragment is human and is derived from an IgG, IgA, IgD, IgM, or IgE. For example, the scFc fragment may be derived from an IgG, such as IgG1, IgG2, IgG3, or IgG4, such as IgG1 scFc fragment.

The scFc fragment may comprise one or more mutations or sets of mutations that modulate the half-life of the fusion protein from, for example, minutes or hours to several days, weeks, or months. For example, the scFc fragment may comprise a mutation at one or more of L234, L235, G236, G237, M252, I253, S254, T256, P329, A330, M428, N434, or a combination thereof (wherein numbering is according to the EU index), such as M428L and N434S (“LS”); M252Y, S254T and T256E (“YTE”); L234A and L235A (“LALA”); I253A; L234A, L235A, and P329G (“LALAP”); G236R; G237A; and/or A330L or a combination thereof.

Moreover, other substitutions in the fusion proteins and nanocages described herein are contemplated, including Fc sequence modifications and addition of other agents (e.g. human serum albumin, human serum albumin peptide sequences and antibodies such as Fabs and/or nanobodies targeting human serum albumin), that allow changes in bioavailability and will be understood by a skilled person. Furthermore, the fusion proteins and nanocages described herein can be modulated in sequence or by addition of other agents to mute immunogenicity and anti-drug responses (therapeutic, e.g. matching sequence to host, or addition of immunosuppressive therapies [such as, for example, methotrexate when administering infliximab for treating rheumatoid arthritis or induction of neonatal tolerance, which is a primary strategy in reducing the incidence of inhibitors against FVIII (reviewed in: DiMichele D M, Hoots W K, Pipe S W, Rivard G E, Santagostino E. International workshop on immune tolerance induction: consensus recommendations. Haemophilia. 2007; 13:1-22, incorporated herein by reference in its entirety]).

As described herein, the nanocage monomer may be formed from subunits. Thus the nanocage monomer subunit may comprise a first apoferritin subunit, optionally a first human apoferritin subunit, such as an apoferritin N- or C-subunit, and wherein the first apoferritin subunit is capable of self-assembling with a second apoferritin subunit, such as a complementary C- or N-subunit. It will be understood that other nanocage monomers can be divided into bipartite subunits much like apoferritin as described herein so that the subunits self-assemble and are each amenable to fusion with a bioactive moiety.

The “N” region of apoferritin typically comprises or consists of a sequence at least 70% (such as at least 75%, 80%, 85%, 90%, 95%, 96%, 97%, 98%, 99%, or 100%) identical to:

(SEQ ID NO: 1)
MSSQIRQNYSTDVEAAVNSLVNLYLQASYTYLSLGFYFDRDDVALEGVS
HFFRELAEEKREGYERLLKMQNQRGGRALFQDIKKPAEDEW.

The “C” region of apoferritin typically comprises or consists of a sequence at least 70% (such as at least 75%, 80%, 85%, 90%, 95%, 96%, 97%, 98%, 99%, or 100%) identical to:

(SEQ ID NO: 2)
GKTPDAMKAAMALEKKLNQALLDLHALGSARTDPHLCDFLETHFLDEEV
KLIKKMGDHLTNLHRLGGPEAGLGEYLFERLTLRHD.

In cases where the antibody or fragment thereof comprises two chains, such as a first and second chain, or a heavy and light chain, the two chains are optionally separated by a linker. The linker may be flexible or rigid, but it typically is flexible to allow the chains to fold appropriately. Similarly, the scFc fragment and/or the bioactive moiety is typically linked to the nanocage monomer or subunit thereof through a linker.

The linker is generally long enough to impart some flexibility to the fusion protein, although it will be understood that linker length will vary depending upon the nanocage monomer or subunit thereof and scFc fragment and bioactive moiety sequences and the three-dimensional conformation of the fusion protein. Thus, the linker is typically from about 1 to about 130 amino acid residues, such as from about 1, 5, 10, 15, 20, 25, 30, 35, 40, 45, 50, 55, 60, 65, 70, 75, 80, 85, 90, 95, 100, 105, 110, 115, 120, or 125 to about 5, 10, 15, 20, 25, 30, 35, 40, 45, 50, 55, 60, 65, 70, 75, 80, 85, 90, 95, 100, 105, 110, 115, 120, 125, or 130 amino acid residues, or from about 50 to about 90 amino acid residues, such as 70 amino acid residues, or from about 1 to about 100 amino acid residues, such as from about 1 to about 70 amino acid residues, such as from about 1 to about 30 amino acid residues, such as from about 8 to about 16 amino acid residues.

The linker may be of any amino acid sequence and, in one typical example, the linker comprises a GS domain, or a series of G and S amino acids, such as a series of GS repeats, GGS repeats, GGGS repeats, and/or GGGGS repeats. Typically, the linker comprises a GGGGS and/or GGGS repeat and, more typically, the linker comprises at least about 2, 3, 4, 5, 6, 7, 8, 9, 10, 11, 12, 13, 14, 15, 16, 17, 18, 19, or 20 GGGS (SEQ ID NO:11) and/or GGGGS (SEQ ID NO:12) repeats, such as about 5 GGGS (SEQ ID NO:11) repeats and/or about 14 GGGGS (SEQ ID NO:12) repeats. In specific aspects, the linker comprises or consists of a sequence at least 70% (such as at least 75%, 80%, 85%, 90%, 95%, 96%, 97%, 98%, 99%, or 100%) identical to:

(SEQ ID NO: 3)
GGGGSGGGGSGGGGSGGGGSGGGGSGGGGSGGGGSGGGGSGGGGSGGGG
SGGGGSGGGGSGGGGSGGGGS,
(SEQ ID NO: 4)
GGGGSGGGGSGGGGSGGGGSGGGGSGG,
or
(SEQ ID NO: 5)
GGSGGSGGSGGSGGGSGGSGGSGGSG.

In typical aspects, the antibody or fragment thereof binds specifically to an antigen associated with HIV-1.

In aspects, the fusion protein described herein comprises or consists of sequences at least 70% (such as at least 75%, 80%, 85%, 90%, 95%, 96%, 97%, 98%, 99%, or 100%) identical to one or more of the following sequences, wherein the ferritin subunit is in bold and linkers are underlined. While in each of these cases either the full ferritin monomer is shown (MSSQIRQNYSTDVEAAVNSLVNLYLQASYTYLSLGFYFDRDDVALEGVSHFFRELAEEKREGYERLL KMQNQRGGRALFQDIKKPAEDEWGKTPDAMKAAMALEKKLNQALLDLHALGSARTDPHLCDFLETH FLDEEVKLIKKMGDHLTNLHRLGGPEAGLGEYLFERLTLRHD; SEQ ID NO:6), or the N- or C-ferritin monomer is shown (MSSQIRQNYSTDVEAAVNSLVNLYLQASYTYLSLGFYFDR DDVALEGVSHFFRELAEEKREGYERLLKMQNQRGGRALFQDIKKPAEDEW (SEQ ID NO:1) in the case of N-ferritin and GKTPDAMKAAMALEKKLNQALLDLHALGSARTDPHLCDFLETHFLDEEVKLIK KMGDHLTNLHRLGGPEAGLGEYLFERLTLRHD (SEQ ID NO:2) in the case of C-ferritin), it will be understood that these could be interchangeably used or another monomer or part thereof could be used in its place.

### 32-N MB.v2:

N49P7_N-Ferritin
(SEQ ID NO: 7)
QSALTQPRSVSASPGQSVTISCTGTHNLVSWCQHQPGRAPKLLIYDFNKRPSGVPDRFSGS
GSGGTASLTITGLQDDDDAEYFCWAYEAFGGGTKLTVLGQPKAAPSVTLFPPSSEELQANKATLVCLI
SDFYPGAVTVAWKADSSPVKAGVETTTPSKQSNNKYAASSYLSLTPEQWKSHRSYSCQVTHEGSTV
EKTVAPTECGGGGSGGGGSGGGGSGGGGSGGGGSGGGGSGGGGSGGGGSGGGGSGGGGSG
GGGSGGGGSGGGGSGGGGSADLVQSGAVVKKPGDSVRISCEAQGYRFPDYIIHWIRRAPGQGPE
WMGWMNPMGGQVNIPWKFQGRVSMTRDTSIETAFLDLRGLKSDDTAVYYCVRDRSNGSGKRFES
SNWFLDLWGRGTAVTIQSASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDKKVEPKSCGGGGSGGGGSG
GGGSGGGGSGGGGSGGMSSQIRQNYSTDVEAAVNSLVNLYLQASYTYLSLGFYFDRDDVALEGV
SHFFRELAEEKREGYERLLKMQNQRGGRALFQDIKKPAEDEW
PGDM1400_hFerritin
(SEQ ID NO: 8)
DFVLTQSPHSLSVTPGESASISCKSSHSLIHGDRNNYLAWYVQKPGRSPQLLIYLASSRASG
VPDRFSGSGSDKDFTLKISRVETEDVGTYYCMQGRESPWTFGQGTKVDIKRTVAAPSVFIFPPSDEQ
LKSGTASVVCLLNNFYPREAKVQWKVDNALQSGNSQESVTEQDSKDSTYSLSSTLTLSKADYEKHKV
YACEVTHQGLSSPVTKSFNRGECGGGGSGGGGSGGGGSGGGGSGGGGSGGGGSGGGGSGGG
GSGGGGSGGGGSGGGGSGGGGSGGGGSGGGGSQAQLVQSGPEVRKPGTSVKVSCKAPGNTLK
TYDLHWVRSVPGQGLQWMGWISHEGDKKVIVERFKAKVTIDWDRSTNTAYLQLSGLTSGDTAVYYC
AKGSKHRLRDYALYDDDGALNWAVDVDYLSNLEFWGQGTAVTVSSASTKGPSVFPLAPSSKSTSGG
TAALGCLVKDYFPEPVTVSWNSGALTSGVHTFPAVLQSSGLYSLSSVVTVPSSSLGTQTYICNVNHK
PSNTKVDKKVEPKSCDGGGGSGGGGSGGGGSGGGGSGGGGSGGMSSQIRQNYSTDVEAAVNSL
VNLYLQASYTYLSLGFYFDRDDVALEGVSHFFRELAEEKREGYERLLKMQNQRGGRALFQDIKKP
AEDEWGKTPDAMKAAMALEKKLNQALLDLHALGSARTDPHLCDFLETHFLDEEVKLIKKMGDHLT
NLHRLGGPEAGLGEYLFERLTLRHD
10E8v4_C-Ferritin_Fc
(SEQ ID NO: 13)
SELTQDPAVSVALKQTVTITCRGDSLRSHYASWYQKKPGQAPVLLFYGKNNRPSGIPDRFS
GSASGNRASLTITGAQAEDEADYYCSSRDKSGSRLSVFGGGTKLTVLSQPKAAPSVTLFPPSSEELQ
ANKATLVCLISDFYPGAVTVAWKADSSPVKAGVETTTPSKQSNNKYAASSYLSLTPEQWKSHRSYSC
QVTHEGSTVEKTVAPTECGGGGSGGGGSGGGGSGGGGSGGGGSGGGGSGGGGSGGGGSGGG
GSGGGGSGGGGSGGGGSGGGGSGGGGSEVRLVESGGGLVKPGGSLRLSCSASGFDFDNAWMT
WVRQPPGKGLEWVGRITGPGEGWSVDYAESVKGRFTISRDNTKNTLYLEMNNVRTEDTGYYFCAR
TGKYYDFWSGYPPGEEYFQDWGQGTLVIVSSASTKGPSVFPLAPSSKSTSGGTAALGCLVKDYFPE
PVTVSWNSGALTSGVHTFPAVLQSSGLYSLSSVVTVPSSSLGTQTYICNVNHKPSNTKVDKKVEPKS
CGGGGSGGGGSGGGGSGGGGSGGGGSGGKTPDAMKAAMALEKKLNQALLDLHALGSARTDPH
LCDFLETHFLDEEVKLIKKMGDHLTNLHRLGGPEAGLGEYLFERLTLRHDGGGGSGGGGSGGGGS
GGGGSGGGGSGDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPVLDSDGSFFLYSK
LTVDKSRWQQGNVFSCSVMHEALHNHYTQKSLSLSPGKGGGGSGGGGSGGGGSGGGGSGGGGS
GGGGSGGGGSGGGGSGGGGSGGGGSGGGGSGGGGSGGGGSGGGGSDKTHTCPPCPAPELLG
GPSVFLFPPKPKDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPSREEMTKNQVSLTCLVK
GFYPSDIAVEWESNGQPENNYKTTPPVLDSDGSFFLYSKLTVDKSRWQQGNVFSCSVMHEALHNHY
TQKSLSLSPGK;

or combinations thereof.

In further aspects, the fusion protein is conjugated to or associated with a further moiety, such as a detectable moiety (e.g., a small molecule, fluorescent molecule, radioisotope, or magnetic particle), a pharmaceutical agent, a diagnostic agent, or combinations thereof and may comprise, for example, an antibody-drug conjugate.

In aspects wherein the further moiety is a detectable moiety, the detectable moiety may comprise a fluorescent protein, such as GFP, EGFP, Ametrine, and/or a flavin-based fluorescent protein, such as a LOV-protein, such as iLOV.

In aspects wherein the further moiety is a pharmaceutical agent, the pharmaceutical agent may comprise for example, a small molecule, a peptide, a lipid, a carbohydrate, or a toxin.

In typical aspects, the nanocage assembled from the fusion proteins described herein comprises from about 3 to about 100 nanocage monomers, none, some, or all of which may be provided as bipartite nanocage monomer subunits, such as from about 3, 4, 5, 6, 7, 8, 9, 10, 12, 14, 16, 18, 20, 22, 24, 26, 28, 30, 32, 34, 36, 38, 40, 42, 44, 46, 48, 50, 52, 55, 56, 58, 60, 62, 64, 66, 68, 70, 72, 74, 76, 78, 80, 82, 84, 86, 88, 90, 92, 94, 96, or 98 to about 4, 5, 6, 7, 8, 9, 10, 12, 14, 16, 18, 20, 22, 24, 26, 28, 30, 32, 34, 36, 38, 40, 42, 44, 46, 48, 50, 52, 55, 56, 58, 60, 62, 64, 66, 68, 70, 72, 74, 76, 78, 80, 82, 84, 86, 88, 90, 92, 94, 96, 98, or 100 nanocage monomers, such as 24, 32, or 60 nanocage monomers. The nanocage monomer or subunit thereof may be any known nanocage monomer, natural, synthetic, or partly synthetic and is, in aspects, selected from ferritin, apoferritin, encapsulin, SOR, lumazine synthase, pyruvate dehydrogenase, carboxysome, vault proteins, GroEL, heat shock protein, E2P, MS2 coat protein, fragments thereof, and variants thereof. Typically, the nanocage monomer or subunit thereof is ferritin or apoferritin or a subunit thereof.

Also described herein are pairs of the fusion proteins described above, wherein each pair self-assembles to form a nanocage monomer, wherein the first and second nanocage monomer subunits are fused to different bioactive moieties as described herein. This provides multivalency and/or multispecificity to a single nanocage monomer assembled from the pair of subunits.

### Nanocages

Also disclosed herein are nanocages comprising at least one fusion protein as disclosed herein, wherein the nanocage self-assembles from the at least one fusion protein and additional fusion protein(s) and/or nanocage monomer(s) or subunits thereof, such as ferritin chain(s) (e.g., human ferritin light chains).

Also described herein are nanocages comprising at least one fusion protein described herein and at least one nanocage monomer or subunit thereof that self-assembles with the fusion protein to form a nanocage. Further, pairs of the fusion proteins are described herein, wherein the pair self-assembles to form a nanocage monomer and wherein the first and second nanocage monomer subunits are fused to different bioactive moieties.

It will be understood that the nanocages may self-assemble from multiple identical fusion proteins, from multiple different fusion proteins (and therefore be multivalent and/or multispecific), from a combination of fusion proteins and wild-type proteins, and any combination thereof. For example, the nanocages may be decorated internally and/or externally with at least one of the fusion proteins described herein in combination with at least one Fc monomer and/or scFc fragment. In some aspects, at least one Fc monomer and/or scFc fragment and at least one Fab fragment decorate the exterior surface of the nanocage. In some aspects, at least two Fc monomers and/or scFc fragments and at least two Fab fragment decorate the exterior surface of the nanocage.

In typical aspects, from about 20% to about 80% of the nanocage monomers or subunits thereof comprise the fusion protein described herein. In view of the modular solution described herein, the nanocages could in theory comprise up to three or four times as many bioactive moieties, such as antibody fragments, and/or Fc monomers/scFc fragments as there are monomers in the nanocage, as each nanocage monomer may be divided into two subunits, each of which can independently bind to a different bioactive moiety at each termini. Despite this, lower numbers are typically used to avoid steric hindrance. It will be understood that this modularity can be harnessed to achieve any desired ratio of bioactive moieties.

In some examples, the nanocages described herein may comprise at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 or more identical or substantially identical or functionally equivalent copies of an Fc monomer or a scFc fragment. In additional or alternative examples, the nanocages described herein may comprise at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 or more identical or substantially identical or functionally equivalent copies of a bioactive moiety, such as an Fab fragment. In additional or alternative examples, the nanocages described herein may comprise at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 different Fc monomers and/or scFc fragments and/or other bioactive moieties. In this way, the nanocages can be multivalent and/or multispecific and the extent of this can be controlled with relative ease with the systems described herein. In some embodiments, the nanocages are both multivalent and multispecific.

In some aspects, the nanocages described herein may further comprise at least one whole nanocage monomer, optionally fused to a bioactive moiety that may be the same or different from the bioactive moiety described herein as being linked to a nanocage monomer subunit.

In some aspects, the nanocages described herein comprise a first and second fusion protein each comprising a different antigen-binding moiety fused to a nanocage monomer or subunit thereof, and optionally a third fusion protein comprising an Fc monomer or scFc fragment, fused to a nanocage monomer or subunit thereof.

In some embodiments, the first, second, and third fusion proteins each comprise a bioactive moiety, or portions thereof, fused to N- or C-half ferritin, wherein at least one of the first, second, and third fusion proteins is fused to N-half ferritin and at least one of the first, second, and third fusion proteins is fused to C-half ferritin and wherein an Fc monomer or an scFc fragment is fused to the C-terminal end of C-half ferritin.

In some embodiments, the first and optionally second fusion proteins each comprise antigen-binding moieties fused to full apoferritin. Similarly, in some embodiments, the third protein comprises the bioactive moiety fused to full apoferritin. It will be understood that combinations of full nanocage monomers and subunits of nanocage monomers are contemplated for use in the modular nanocages described herein.

It will be understood that typically at least one full nanocage monomer linked to a bioactive moiety, such as an Fab, at its N-terminus is used in the nanocages described herein. Typically, there is one such full nanocage monomer per pair of N- and C-ferritin subunits. This permits the nanocage to multimerize properly without excessive steric hindrance and also lowers the number of Fc monomers and/or scFc fragments on the nanocages that might lead to undesired excessive avidity.

While the proteins can comprise any numbers or ratios of fusion proteins, in some embodiments, the nanocage described herein comprises three Fabs, each specific for a different antigen associated with the same disease, and a Fc monomer or scFc fragment, optionally in a Fab1:Fab2:Fab:Fc monomer/scFc ratio of 2:2:2:1.

Also described herein are compositions comprising the nanocage, such as therapeutic or prophylactic compositions. Related methods and uses for treating and/or preventing HIV-1 are also described, wherein the method or use comprises administering the nanocage or composition described herein to a subject in need thereof.

In specific embodiments directed to HIV-1, the nanocage in aspects exhibits pan-virus neutralization breadth. In additional or alternative aspects, the nanocage exhibits an average median IC₅₀ value against a multiclade panel of 118 pseudoviruses (PsV) of less than about 0.1 ug/mL, such as less than about 0.01 ug/mL, such as less than about 0.001 ug/mL. In additional or alternative aspects, the nanocage exhibits an average median IC₅₀ value against a multiclade panel of 118 pseudoviruses (PsV) of less than about 42 pM, such as less than about 4.2 pM, such as less than about 0.42 pM. In additional or alternative aspects, the nanocage exhibits an average median IC₅₀ value against a multiclade panel of 118 PsVs that is at least about 10, at least about 100, at least about 1000, at least about 10,000, or at least about 100,000 more potent than a cocktail of the corresponding bNAbs on a mass and/or molar basis.

It will be understood that polypeptides substantially identical to those described herein are also contemplated. A substantially identical sequence may comprise one or more conservative amino acid mutations. It is known in the art that one or more conservative amino acid mutations to a reference sequence may yield a mutant peptide with no substantial change in physiological, chemical, or functional properties compared to the reference sequence; in such a case, the reference and mutant sequences would be considered “substantially identical” polypeptides. Conservative amino acid mutation may include addition, deletion, or substitution of an amino acid; a conservative amino acid substitution is defined herein as the substitution of an amino acid residue for another amino acid residue with similar chemical properties (e.g. size, charge, or polarity).

In a non-limiting example, a conservative mutation may be an amino acid substitution. Such a conservative amino acid substitution may substitute a basic, neutral, hydrophobic, or acidic amino acid for another of the same group. By the term “basic amino acid” it is meant hydrophilic amino acids having a side chain pK value of greater than 7, which are typically positively charged at physiological pH. Basic amino acids include histidine (His or H), arginine (Arg or R), and lysine (Lys or K). By the term “neutral amino acid” (also “polar amino acid”), it is meant hydrophilic amino acids having a side chain that is uncharged at physiological pH, but which has at least one bond in which the pair of electrons shared in common by two atoms is held more closely by one of the atoms. Polar amino acids include serine (Ser or S), threonine (Thr or T), cysteine (Cys or C), tyrosine (Tyr or Y), asparagine (Asn or N), and glutamine (Gln or Q). The term “hydrophobic amino acid” (also “non-polar amino acid”) is meant to include amino acids exhibiting a hydrophobicity of greater than zero according to the normalized consensus hydrophobicity scale of Eisenberg (1984). Hydrophobic amino acids include proline (Pro or P), isoleucine (lie or I), phenylalanine (Phe or F), valine (Val or V), leucine (Leu or L), tryptophan (Trp or W), methionine (Met or M), alanine (Ala or A), and glycine (Gly or G).

“Acidic amino acid” refers to hydrophilic amino acids having a side chain pK value of less than 7, which are typically negatively charged at physiological pH. Acidic amino acids include glutamate (Glu or E), and aspartate (Asp or D).

Sequence identity is used to evaluate the similarity of two sequences; it is determined by calculating the percent of residues that are the same when the two sequences are aligned for maximum correspondence between residue positions. Any known method may be used to calculate sequence identity; for example, computer software is available to calculate sequence identity. Without wishing to be limiting, sequence identity can be calculated by software such as NCBI BLAST2 service maintained by the Swiss Institute of Bioinformatics (and as found at ca.expasy.org/tools/blast/), BLAST-P, Blast-N, or FASTA-N, or any other appropriate software that is known in the art.

The substantially identical sequences of the present invention may be at least 85% identical; in another example, the substantially identical sequences may be at least 70, 75, 80, 85, 90, 95, 96, 97, 98, 99, or 100% (or any percentage there between) identical at the amino acid level to sequences described herein. In specific aspects, the substantially identical sequences retain the activity and specificity of the reference sequence. In a non-limiting embodiment, the difference in sequence identity may be due to conservative amino acid mutation(s).

The polypeptides or fusion proteins of the present invention may also comprise additional sequences to aid in their expression, detection, purification, or any other desired properties, such as peptides that inhibit transcytosis. Any such sequences or tags known to those of skill in the art may be used. For example, and without wishing to be limiting, the fusion proteins may comprise a targeting or signal sequence (for example, but not limited to ompA), a detection tag, exemplary tag cassettes include Strep tag, or any variant thereof; see, e.g., U.S. Pat. No. 7,981,632, His tag, Flag tag having the sequence motif DYKDDDDK (SEQ ID NO:14), Xpress tag, Avi tag, Calmodulin tag, Polyglutamate tag, HA tag, Myc tag, Nus tag, S tag, SBP tag, Softag 1, Softag 3, V5 tag, CREB-binding protein (CBP), glutathione S-transferase (GST), maltose binding protein (MBP), green fluorescent protein (GFP), Thioredoxin tag, or any combination thereof; a purification tag (for example, but not limited to a Hiss or His₆), or a combination thereof.

In another example, the additional sequence may be a biotin recognition site such as that described by Cronan et al in WO/1995/004069 or Voges et al in WO/2004/076670. As is also known to those of skill in the art, linker sequences may be used in conjunction with the additional sequences or tags.

More specifically, a tag cassette may comprise an extracellular component that can specifically bind to an antibody with high affinity or avidity. Within a single chain fusion protein structure, a tag cassette may be located (a) immediately amino-terminal to a connector region, (b) interposed between and connecting linker modules, (c) immediately carboxy-terminal to a binding domain, (d) interposed between and connecting a binding domain (e.g., scFv or scFab) to an effector domain, (e) interposed between and connecting subunits of a binding domain, or (f) at the amino-terminus of a single chain fusion protein. In certain embodiments, one or more junction amino acids may be disposed between and connecting a tag cassette with a hydrophobic portion, or disposed between and connecting a tag cassette with a connector region, or disposed between and connecting a tag cassette with a linker module, or disposed between and connecting a tag cassette with a binding domain.

Also encompassed herein are isolated or purified fusion proteins, polypeptides, or fragments thereof immobilized onto a surface using various methodologies; for example, and without wishing to be limiting, the polypeptides may be linked or coupled to the surface via His-tag coupling, biotin binding, covalent binding, adsorption, and the like. The solid surface may be any suitable surface, for example, but not limited to the well surface of a microtiter plate, channels of surface plasmon resonance (SPR) sensor chips, membranes, beads (such as magnetic-based or sepharose-based beads or other chromatography resin), glass, a film, or any other useful surface.

In other aspects, the fusion proteins may be linked to a cargo molecule; the fusion proteins may deliver the cargo molecule to a desired site and may be linked to the cargo molecule using any method known in the art (recombinant technology, chemical conjugation, chelation, etc.). The cargo molecule may be any type of molecule, such as a therapeutic or diagnostic agent.

In some aspects, the cargo molecule is a protein and is fused to the fusion protein such that the cargo molecule is contained in the nanocage internally or externally. In other aspects, the cargo molecule is not fused to the fusion protein and is contained in the nanocage internally. The cargo molecule is typically a protein, a small molecule, a radioisotope, or a magnetic particle.

The fusion proteins described herein specifically bind to their targets. Antibody specificity, which refers to selective recognition of an antibody for a particular epitope of an antigen, of the antibodies or fragments described herein can be determined based on affinity and/or avidity. Affinity, represented by the equilibrium constant for the dissociation of an antigen with an antibody (K_{D}), measures the binding strength between an antigenic determinant (epitope) and an antibody binding site. Avidity is the measure of the strength of binding between an antibody with its antigen. Antibodies typically bind with a K_{D} of 10⁻⁵ to 10⁻¹¹ M. Any K_{D} greater than 10⁻⁴ M is generally considered to indicate non-specific binding. The lesser the value of the K_{D}, the stronger the binding strength between an antigenic determinant and the antibody binding site. In aspects, the antibodies described herein have a K_{D} of less than 10⁻⁴ M, 10⁻⁵ M, 10⁻⁶ M, 10⁻⁷ M, 10⁻³ M, 10⁻⁹ M, 10⁻¹⁰ M, 10⁻¹¹ M, 10⁻¹² M, 10⁻¹³ M, 10⁻¹⁴ M, or 10⁻¹⁵ M.

Also described herein are nucleic acid molecules encoding the fusion proteins and polypeptides described herein, as well as vectors comprising the nucleic acid molecules and host cells comprising the vectors.

Polynucleotides encoding the fusion proteins described herein include polynucleotides with nucleic acid sequences that are substantially the same as the nucleic acid sequences of the polynucleotides of the present invention. “Substantially the same” nucleic acid sequence is defined herein as a sequence with at least 70%, at least 75%, at least 80%, at least 85%, at least 90%, at least 91%, at least 92%, at least 93%, at least 94%, at least 95% identity to another nucleic acid sequence when the two sequences are optimally aligned (with appropriate nucleotide insertions or deletions) and compared to determine exact matches of nucleotides between the two sequences.

Suitable sources of polynucleotides that encode fragments of antibodies include any cell, such as hybridomas and spleen cells, that express the full-length antibody. The fragments may be used by themselves as antibody equivalents, or may be recombined into equivalents, as described above. The DNA deletions and recombinations described in this section may be carried out by known methods, such as those described in the published patent applications listed above in the section entitled “Functional Equivalents of Antibodies” and/or other standard recombinant DNA techniques, such as those described below. Another source of DNAs are single chain antibodies produced from a phage display library, as is known in the art.

Additionally, expression vectors are provided containing the polynucleotide sequences previously described operably linked to an expression sequence, a promoter and an enhancer sequence. A variety of expression vectors for the efficient synthesis of antibody polypeptide in prokaryotic, such as bacteria and eukaryotic systems, including but not limited to yeast and mammalian cell culture systems have been developed. The vectors of the present invention can comprise segments of chromosomal, non-chromosomal and synthetic DNA sequences.

Any suitable expression vector can be used. For example, prokaryotic cloning vectors include plasmids from E. coli, such as colEI, pCRI, pBR322, pMB9, pUC, pKSM, and RP4. Prokaryotic vectors also include derivatives of phage DNA such as M13 and other filamentous single-stranded DNA phages. An example of a vector useful in yeast is the 2μ plasmid. Suitable vectors for expression in mammalian cells include well-known derivatives of SV-40, adenovirus, retrovirus-derived DNA sequences and shuttle vectors derived from combination of functional mammalian vectors, such as those described above, and functional plasmids and phage DNA.

Additional eukaryotic expression vectors are known in the art (e.g., P J. Southern & P. Berg, J. Mol. Appl. Genet, 1:327-341 (1982); Subramani et al, Mol. Cell. Biol, 1: 854-864 (1981); Kaufman & Sharp, “Amplification And Expression of Sequences Cotransfected with a Modular Dihydrofolate Reductase Complementary DNA Gene,” J. Mol. Biol, 159:601-621 (1982); Kaufman & Sharp, Mol. Cell. Biol, 159:601-664 (1982); Scahill et al., “Expression And Characterization Of The Product Of A Human Immune Interferon DNA Gene In Chinese Hamster Ovary Cells,” Proc. Nat'l Acad. Sci USA, 80:4654-4659 (1983); Urlaub & Chasin, Proc. Nat'l Acad. Sci USA, 77:4216-4220, (1980), all of which are incorporated by reference herein).

The expression vectors typically contain at least one expression control sequence that is operatively linked to the DNA sequence or fragment to be expressed. The control sequence is inserted in the vector in order to control and to regulate the expression of the cloned DNA sequence. Examples of useful expression control sequences are the lac system, the trp system, the tac system, the trc system, major operator and promoter regions of phage lambda, the control region of fd coat protein, the glycolytic promoters of yeast, e.g., the promoter for 3-phosphoglycerate kinase, the promoters of yeast acid phosphatase, e.g., Pho5, the promoters of the yeast alpha-mating factors, and promoters derived from polyoma, adenovirus, retrovirus, and simian virus, e.g., the early and late promoters or SV40, and other sequences known to control the expression of genes of prokaryotic or eukaryotic cells and their viruses or combinations thereof.

Also described herein are recombinant host cells containing the expression vectors previously described. The fusion proteins described herein can be expressed in cell lines other than in hybridomas. Nucleic acids, which comprise a sequence encoding a polypeptide according to the invention, can be used for transformation of a suitable mammalian host cell.

Cell lines of particular preference are selected based on high level of expression, constitutive expression of protein of interest and minimal contamination from host proteins. Mammalian cell lines available as hosts for expression are well known in the art and include many immortalized cell lines, such as but not limited to, HEK 293 cells, Chinese Hamster Ovary (CHO) cells, Baby Hamster Kidney (BHK) cells and many others. Suitable additional eukaryotic cells include yeast and other fungi. Useful prokaryotic hosts include, for example, E. coli, such as E. coli SG-936, E. coli HB 101, E. coli W3110, E. coli X1776, E. coli X2282, E. coli DHI, E. coli T7 Shuffle and E. coli MRC1, Pseudomonas, Bacillus, such as Bacillus subtilis, and Streptomyces.

These present recombinant host cells can be used to produce fusion proteins by culturing the cells under conditions permitting expression of the polypeptide and purifying the polypeptide from the host cell or medium surrounding the host cell. Targeting of the expressed polypeptide for secretion in the recombinant host cells can be facilitated by inserting a signal or secretory leader peptide-encoding sequence (See, Shokri et al, (2003) Appl Microbiol Biotechnol. 60(6): 654-664, Nielsen et al, Prot. Eng., 10:1-6 (1997); von Heinje et al., Nucl. Acids Res., 14:4683-4690 (1986), all of which are incorporated by reference herein) at the 5′ end of the antibody-encoding gene of interest. These secretory leader peptide elements can be derived from either prokaryotic or eukaryotic sequences. Accordingly suitably, secretory leader peptides are used, being amino acids joined to the N-terminal end of a polypeptide to direct movement of the polypeptide out of the host cell cytosol and secretion into the medium.

The fusion proteins described herein can be fused to additional amino acid residues. Such amino acid residues can be a peptide tag to facilitate isolation, for example. Other amino acid residues for homing of the antibodies to specific organs or tissues are also contemplated.

It will be understood that a Fab-nanocage can be generated, e.g., by co-transfection of plasmids, one encoding a fusion protein comprising an Fab heavy chain fused to a ferritin chain (e.g., ferritin light chain), and another encoding an Fab light chain. Typically, the constructs are arranged as LC-linker-HC-linker-nanocage monomer/subunit, but could also be arranged HC-linker-LC-linker-nanocage monomer/subunit. As described herein, scFv or any other antigen-binding moieties could be used. Alternatively, single-chain Fab-ferritin nanocages can be used that only require transfection of one plasmid (e.g., using a plasmid that encodes a fusion protein comprising Fab light chain, Fab heavy chain, and a ferritin chain (e.g., ferritin light chain)). This can be done with linkers of different lengths between the Fab light chain and the Fab heavy chain for example 60 or 70 amino acids. When single-chain Fabs are used, it can be ensured that the heavy chain and light chain are paired. Tags (e.g. Flag, HA, myc, His6x, Strep, etc.) can also be added at the N terminus of the construct or within the linker for ease of purification as described above. Further, a tag system can be used to make sure many different Fabs are present on the same nanoparticle using serial/additive affinity chromatography steps when different Fab-nanoparticle plasmids are co-transfected. This provides multi-specificity to the nanoparticles. Protease sites (e.g. TEV, 3C, etc.) can be inserted to cleave linkers and tags after expression and/or purification, if desired.

Any suitable method or route can be used to administer the fusion proteins described herein. Routes of administration include, for example, oral, intranasal, intravenous, intraperitoneal, subcutaneous, or intramuscular administration.

It is understood that the fusion proteins described herein, where used in a mammal for the purpose of prophylaxis or treatment, will be administered in the form of a composition additionally comprising a pharmaceutically acceptable carrier. Suitable pharmaceutically acceptable carriers include, for example, one or more of water, saline, phosphate buffered saline, dextrose, glycerol, ethanol and the like, as well as combinations thereof. Pharmaceutically acceptable carriers may further comprise minor amounts of auxiliary substances such as wetting or emulsifying agents, preservatives or buffers, which enhance the shelf life or effectiveness of the binding proteins. The compositions of the injection may, as is well known in the art, be formulated so as to provide quick, sustained or delayed release of the active ingredient after administration to the mammal.

Although the fusion peptides and Multabodies described herein are particularly useful for administration to humans, they may be administered to other mammals as well. The term “mammal” as used herein is intended to include, but is not limited to, humans, laboratory animals, domestic pets and farm animals.

The above disclosure generally describes the present invention. A more complete understanding can be obtained by reference to the following specific examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

The following examples do not include detailed descriptions of conventional methods, such as those employed in the construction of vectors and plasmids, the insertion of genes encoding polypeptides into such vectors and plasmids, or the introduction of plasmids into host cells. Such methods are well known to those of ordinary skill in the art and are described in numerous publications including Sambrook, J., Fritsch, E. F. and Maniatis, T. (1989), Molecular Cloning: A Laboratory Manual, 2nd edition, Cold Spring Harbor Laboratory Press, which is incorporated by reference herein.

Without further description, it is believed that one of ordinary skill in the art can, using the preceding description and the following illustrative examples, make and utilize the compounds of the present invention and practice the claimed methods. The following working examples therefore, specifically point out the typical aspects of the present invention, and are not to be construed as limiting in any way the remainder of the disclosure.

### EXAMPLES

### Example 1

### Abstract

The rapid evolution of HIV continues to be a major barrier for the development of therapeutics for prevention and treatment. Here we describe designs of second-generation antibody platforms called Multabody.v2 (MB.v2) and Multabody.v3 (MB.v3) that allow the formation of a single molecule able to establish multiple and simultaneous interactions with three independent epitopes of a target, in this case, the HIV Envelope. The multivalency and multispecificity properties of this technology translates into extraordinary potency surpassing that of the most potent anti-HIV bnAbs and into complete pan-neutralization of a range of diverse HIV-1 strains. These new MB.v2 and MB.v3 platforms therefore have potential for prophylaxis and therapy against HIV-1.

### Introduction

Despite decades of research, no effective vaccine or cure exist against the human immunodeficiency virus type I (HIV-1). However, the fact that a small proportion of HIV-1 infected individuals develop antibodies with exceptional neutralization potency across circulating HIV-1 isolates highlights the potential for antibody-mediated control of HIV-1. Since the first generation of broadly neutralizing antibodies (bNAbs) 2F5(1), 4E10(2, 3), 2G12(4) and b12(5, 6) were discovered, the catalogue of bNAbs has dramatically increased due to implementation of new technologies of Env-specific single B cell sorting (7-9), antibody cloning and high-throughput neutralization assays (10-13), and more recently proteomic deconvolution (14). Several dozens of HIV bNAbs have now been described to target six conserved sites on the trimeric HIV Envelope (Env), including the V1/V2 loops at the trimer apex, V3 loop glycans, the CD4 binding site (CD4bs), the gp120-g41 interface, the fusion peptide and the membrane-proximal external region (MPER)(7, 9, 10, 12-19).

The interest of bNAbs as therapeutic molecules in the fight against HIV-1 arise from the potent antiviral activity observed for challenge studies in macaques(20-23, 23) and humanized mice (24-27), and from the reduced viremia achieved in infected humans following infusion of bNAbs (28-32). In addition, antibodies possess key advantages in comparison to oral antiretroviral therapy (ART): they have longer circulating half-lives and can form immune complexes that enhance host immunity to the virus. These observations have led to the clinical evaluation of antibody-based therapy to confer protection against HIV-1 through passive administration of bNAbs, and efforts to control and/or clear HIV-1 in infected individuals.

One of the main limitations for the clinical use of bNAbs is the rapid evolution of neutralization-resistant virus populations (29-31, 33, 34). RNA viruses such as HIV-1 exhibit an extraordinary genetic diversity (35) enabling the virus to develop resistant mutations to escape mAb recognition. However, mutations that abrogate binding to certain bNAbs can carry a significant penalty in viral fitness (36-38). Analogous to the combination of different drugs in HIV-1 treatment regimens, this observation suggests that a successful antibody-based therapy against HIV-1 should include a combination of bNAb specificities. As a consequence, the development of different formats of antibody-like molecules with bi (39-41) or tri-specificity (42-44) toward Env has recently been explored. An additional consideration is the amount of antibody required for in vivo efficacy. Indeed, extensive efforts have been directed towards engineering bNAbs to improve their potency using structure-guide design or bioinformatic approaches, such as VRC01(45), 10E8(46, 47) and NIH45-46(48), but so far with moderate success. In the case of bispecific and trispecific antibodies that target multiple epitopes in Env, potency is generally limited by the potency of their parental mAbs. Consequently, a significant improvement in neutralization breadth but relatively little in antiviral potency has so far been achieved^{40,43}(43)⁴⁵.

We have recently described a MULTi-specific, multi-Affinity antiBODY (Multabody) platform to drive oligomerization of antibody fragments and transform antibodies targeting SARS-CoV-2 into exceptionally potent neutralizers(49). Leveraging this platform, we have here combined within a single molecule multiple copies of three of the best bNAbs against HIV-1 and the crystallizable fragment (Fc) from IgG1. By further engineering of the Multabody to maximize the number of Fabs within the particle, we have significantly improved the potency, breadth and homogeneity achieved by the Multabody technology. The resulting anti-HIV Multabody.v3 showed complete pan-virus neutralization breadth and an average median IC₅₀ value against a multiclade panel of 118 pseudoviruses (PsV) of 0.0009 μg/mL (0.4 pM), 32- and 460-fold lower in mass and molarity in comparison to a cocktail made of the best currently know bNAbs, respectively. The Multabody.v3 design described here represents a robust and powerful platform for the development of next-generation biologics against HIV-1.

### Materials and Methods

Expression and purification of Fab-only apoferritin-based multimers. Genes encoding the light chain of human apoferritin and the scFab-human apoferritin fusions were synthesized and cloned by GeneArt (Life Technologies) into the pHLsec expression vector. 200 ml of HEK293F cells (Thermo Fisher Scientifics) were seeded at a density of 0.8 10⁶ cells/mL in Freestyle expression media and incubated with 125 rpm oscillation at 37° C., 8% CO₂, and 70% humidity in a Multitron Pro shaker (Infors HT). Within 24 h after seeding, cells were transiently transfected using 50 μg of filtered DNA preincubated for 10 min at room temperature (RT) with the transfection reagent FectoPRO (Polyplus Transfections) in a 1:1 ratio. Plasmids encoding for scFab-human apoferritin and human apoferritin were mixed in a ratio of 1:4, 1:1, 4:1 and 1:0 in order to obtain 20%, 50%, 80% and 100% scFab valency nanoparticles, respectively. After 6-7 days, cell suspensions were harvested by centrifugation at 5000×g for 15 min and the supernatants filtered through a 0.22 μm Steritop filter (EMD Millipore). The nanoparticles were purified by affinity chromatography to the Fab and eluting after a wash. Fractions containing protein were pooled, concentrated and loaded onto a Superose 6 10/300 GL size exclusion column (GE Heathcare) in 20 mM sodium phosphate pH 8.0, 150 mM NaCl.

Design, expression and purification of 32-N MB.v1, 32-N MB.v2, and 32-N MB.v3 Multabodies. Genes encoding for scFab, scFc and Fc fragments linked to half ferritin were generated by deletion of residues 1 to 95 (leaving a C-terminal portion of ferritin, “C-Ferritin”) or residues 95 to 175 (leaving an N-terminal portion of ferritin, “N-Ferritin”) of the light chain of human apoferritin using the KOD-Plus mutagenesis kit (Toyobo, Osaka, Japan). Transient transfection of the 32-N MB.v1 in HEK 293F cells were obtained by mixing 67 μg of the plasmids PGDM1400 scFab-human apoferritin: scFcN-Ferritin: N49P7 scFab-C-Ferritin: 10E8v4 scFab-C-Ferritin in a 4:2:1:1 ratio. In the case of the 32-N MB.v2, 59 μg of the plasmids PGDM1400 scFab-human apoferritin: N49P7 scFab-N-Ferritin: 10E8v4 scFab-C-Ferritin-scFc in a 5:1:1 ratio was used, and in the case of the 32-N MB.v3, 63 μg of the plasmids PGDM1400 scFab-human apoferritin: N49P7 scFab-N-Ferritin: 10E8v4 scFab-C-Ferritin-Fc in a 3:1:1 ratio were used. Properties of various constructs used in this Example are summarized in Table 1 below.

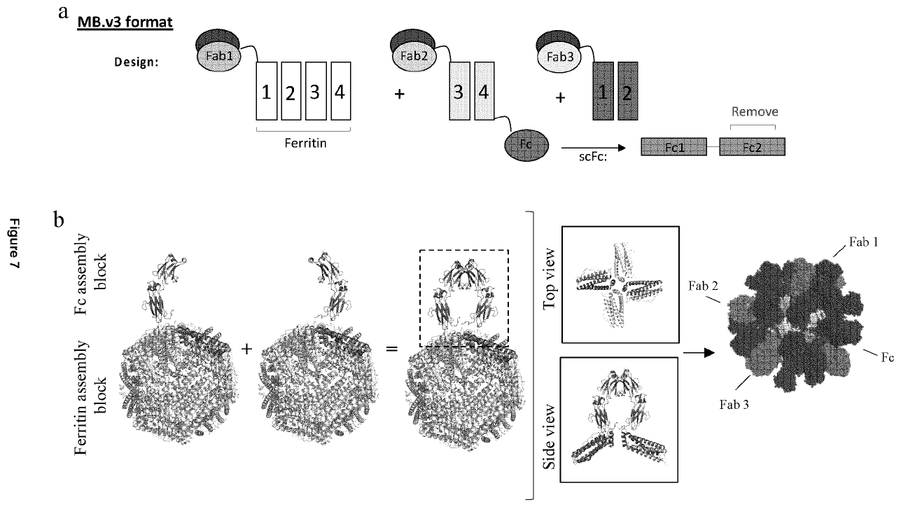

TABLE 1
Properties of Multabody constructs.

| | | | Fab components |
|---|---|---|---|
| Construct and | | Attachment | (see corresponding FIG. |
| corresponding FIG. | Fc | of Fc | for arrangements) |
| 32-N MB.v1 | Single- | N-terminus | Fab1: PGDM1400 scFab |
| (top of FIG. 6A) | chain Fc | of N-ferritin | Fab2: N49P7 scFab |
| (also referred to as | (scFc) | | Fab3: 10E8v4 scFab |
| T-01 MB) | | | |
| 32-N MB.v2 | scFc | C-terminus | Fab1: PGDM1400 scFab |
| (bottom of FIG. 6A) | | of C-ferritin | Fab2: 10E8v4 scFab |
| (also referred to as | | | Fab3: N49P7 scFab |
| T-01 MBv1.5) | | | |
| 32-N MB.v3 | Fc | C-terminus | Fab1: PGDM1400 scFab |
| (FIG. 7A) | monomer | of C-ferritin | Fab2: 10E8v4 scFab |
| (also referred to as | | | Fab3: N49P7 scFab |
| T-01 MBv2) | | | |

The DNA mixture was filtered and incubated at RT with FectoPRO in a 1:1 ratio before adding to the cell culture. Multabodies were purified by affinity chromatography using first a HiTrap Protein A HP column (GE Healthcare) with 20 mM Tris pH 8.0, 3 M MgCl₂ and 10% glycerol elution buffer. After buffer exchange using a PD-10 desalting column (GE Healthcare), Multabodies were further purified by a second affinity chromatography using a HiTrap Protein L column (GE Healthcare). Fractions containing the protein were concentrated and further purified by gel filtration on a Superose 6 10/300 GL column (GE Healthcare)) in 20 mM sodium phosphate pH 8.0, 150 mM NaCl.

Negative-stain electron microscopy. 3 μL of Multabody at a concentration approximately of 0.02 mg/mL was added to a carbon-coated copper grid for 30 s and stained with 3 μL of 2% uranyl formate. Staining excess was immediately removed from the grid using Whatman No. 1 filter paper and an additional 3 μL of 2% uranyl formate was added for 20 s. Grids were imaged using a field-emission FEI Tecnai F20 electron microscope operating at 200 kV and equipped with an Orius charge-coupled device (CCD) camera (Gatan Inc.)

Biolayer interferometry. Binding kinetics measurements were conducted using an Octet RED96 BLI system (Pall ForteBio) in PBS pH 7.4, 0.01% BSA and 0.002% Tween. A unique His-tagged ligand for each of the Multabody components was selected and loaded onto Ni-NTA biosensors to reach a signal response of 0.8 nm. Association rates were measured by transferring the loaded biosensors to wells containing serial dilutions of the multabodies (50-25-12.5-6.25-3.1-1.5 nM) and buffer containing wells, respectively. Dissociation rates were measured by dipping the biosensors into buffer-containing wells. The duration of each of these two steps was 180 s. To achieve selective binding to PGDM1400, a D368R mutation in the CD4bs of the BG505 SOSIP.664 trimer was introduced and consequently, binding of N49P7 to this antigen was disrupted. Similarly, the gp120 subunit 93TH057, MPER peptide and Fc receptors (FcγRI, FcγRIIa, FcγRIIb and hFcRn in complex with β2-microglobulin) were produced as ligands for N49P7, 10E8, and Fc binding respectively. The capacity of the multabodies to undergo endosomal recycling was tested by measuring their binding to the hFcRn β2-microglobulin complex at physiological (7.5) and acidic (5.6) pH.

Size-exclusion chromatography in-line with multi-angle light scattering (SEC-MALS). A MiniDAWN TREOS and an Optilab T-rEX refractometer (Wyatt) were used in-line to an Agilent Technologies 1260 infinity II HPLC. 50 μg of 24-mer PGDM1400 scFab multimer and Multabody 32-N were loaded onto a Superose 6 10/300 (GE Healthcare) column in 20 mM sodium phosphate pH 8.0, 150 mM NaCl. Data collection and analysis were performed using the ASTRA software (Wyatt).

Melting and aggregation temperature measurements. The melting temperature (Tₘ) and aggregation temperature (T_{agg}) of the Multabodies, parental IgGs and the 12-mer homo-oligomeric Fabs and Fc was determined using a UNit system (Unchained Labs). Tₘ was obtained by measuring the barycentric mean fluorescence, while T_{agg} was determined as the temperature at which 50% increase in the static light scattering at a 266 nm wavelength relative to baseline was observed.

Samples were concentrated to 1.0 mg/mL and subjected to a thermal ramp from 25 to 95° C. with 1° C. increments. The average and the standard error of three independent measurements were calculated using the UNit analysis software.

Accelerated thermostability assay. The different Multabody versions were subjected to extreme conditions of temperature and concentration for four consecutive weeks. 10 mg/ml of each sample was incubated at 40° C. and the percentage of properly folded protein was calculated every week analyzing the area of each of the peaks obtained by size exclusion chromatography upon loading 10 μL of the sample on a Superose 6 10/300 GL column (GE Healthcare). This analysis was supplemented with a functional assay, in this case a neutralization assay, to determine the amount of active protein at the beginning (week 0) and at the end of the experiment (week 4).

Virus production and TZM-bl neutralization assays. A panel of 25 HIV-1 pseudotyped viruses was generated by co-transfection of 293T cells with the HIV-1 subtype B backbone NL4-3.Luc.R-E plasmid (AIDS Research and Reference Reagent Program (ARRRP)) and the plasmid encoding the full-length Env clone, as previously described(50). HIV isolates X2088, ZM106.9, NL4.3 and 3817 were kindly provided by the collaboration for AIDS Vaccine Discovery (CAVD), SF162 from J. L. Nieva. (Biofisika Institute) and pCNE8, 1632, THRO, 278, ZM197, JRCSF, t257, Du422, BG505, p1054.TC4.1499, 6535, ZM214M.PL15, AC10.29, p16845, P6244_13.B5.4576, pM246F_C1G, TRJ04551, QH0692 and pCAAN5342 from NIH ARRRP. Neutralization was determined in a single-cycle neutralization assay using the standard TZM-bl neutralization assay. Briefly, antibodies and antibody-based particles were incubated with a 10-15% tissue culture infectious dose of pseudovirus for 1 h at 37° C. prior to a 44-72 h incubation with TZM-bl cells. Virus neutralization was monitored by adding Britelite plus reagent (PerkinElmer) to the cells and measuring luminescence in relative light units (RLUs) using a Synergy Neo2 Multi-Mode Assay Microplate Reader (Biotek Instruments). The extended multiclade panel of 118 PsV was performed at the Center for Virology and Vaccine Research, Harvard Medical School by Dr. Michael Seaman following standard protocols. A cutoff limit of 10 μg/mL was used to determine antibody breadth.

Pharmacokinetics. In vivo studies were performed using female NOD/Shi-scid/lL-2Rγnull immunodeficient mouse strain (NCG). 32-N MB.v3 composed of the scFab of antibodies PGDM1400, N49P7 and 10E8v4 and scFc fragments of IgG1 Fc containing the half-life extension mutations (M428L/N434S) was used for the study. A single injection of 5 mg/kg of the Multabodies or the control samples (an IgG mixture matching the Fab specificity of the Multabody) in 200 μL of PBS (pH 7.5) was subcutaneously injected. Blood samples were collected at multiple time points and serum samples were assessed for levels of circulating antibodies by ELISA. Briefly, 96-well Pierce Nickel Coated Plates (Thermo Fisher) were coated with 50 μL at 0.5 μg/ml of each of the His₆x-tagged antigens recognized by the MB: BG505 SOSIP.664_D368R trimer, gp120 subunit 93TH057 and MPER peptide, to determine circulating sample concentrations using reagent-specific standard curves for IgGs and Multabodies. HRP-Protein A (Invitrogen) was used as a secondary molecule and the chemiluminescence signal was quantified using a Synergy Neo2 Multi-Mode Assay Microplate Reader (Biotek Instruments).

### Results

Potency of HIV-1 bNAbs can be Enhanced with Avidity

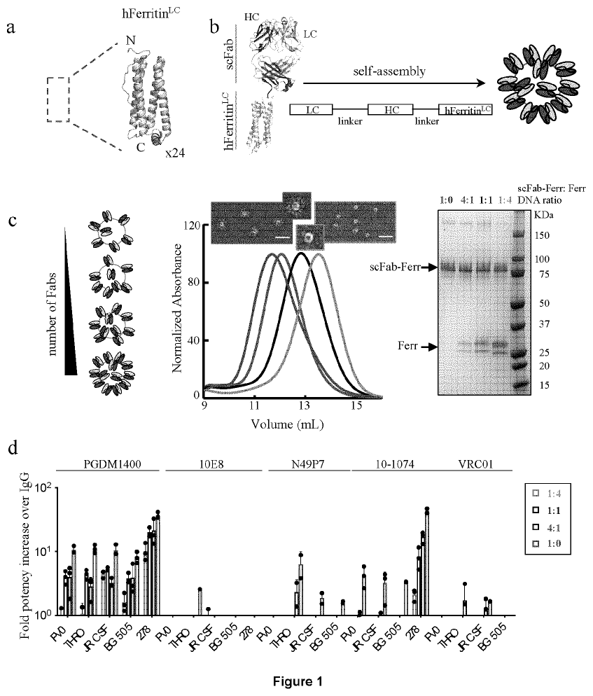

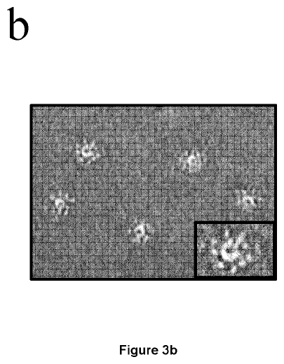

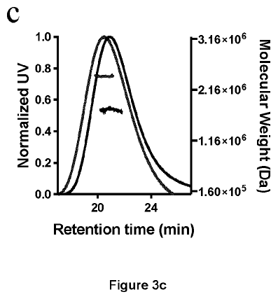

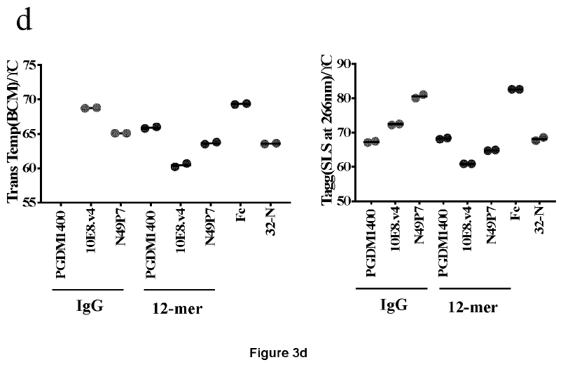

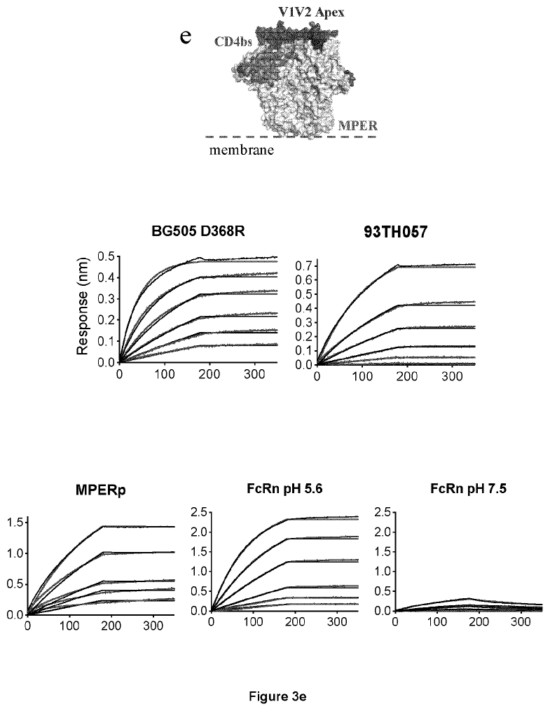

Apoferritin is a spherical nanocage of approximately 6 nm hydrodynamic radius formed by the self-oligomerization of 24 identical subunits (FIG. 1a). To investigate the impact of multi-valency on neutralization potency, we used the self-assembly properties of the light chain of human apoferritin to multimerize fragments of antigen binding (Fabs) derived from the most potent and broad HIV-1 bNAbs, which target different HIV-1 Env epitopes. Apoferritin subunits were genetically fused to single-chain Fabs (scFabs). scFabs were generated using flexible linkers between the light and heavy chains to ensure correct Fab heterodimerization. Apoferritin self-assembly drove multimerization of the scFab and displayed the antibody fragments at the nanocage periphery (FIG. 1b). Different densities of multimerized Fabs were achieved by co-transfection of scFab-human apoferritin-encoding plasmids together with different ratios of non-genetically modified human apoferritin (FIG. 1c and FIG. 3). The ability of the scFab-apoferritin fusions to block HIV-1 infection were compared to the corresponding IgGs using a small HIV-1 pseudovirus (PsV) panel (FIG. 1d). Strikingly, PGDM1400, one of the most potent anti-HIV bNAb described to date, showed 10- to 40-fold higher neutralization potency when multimerized via the light chain of apoferritin compared to its conventional IgG format. bNAb 10-1074 also showed a considerable improvement in neutralization potency (4- to 40-fold), whereas bNAbs 10E8, N49P7, and VRC01 showed no effect or more modest enhancements.

Characterization of scFab-Apoferritin Fusions.

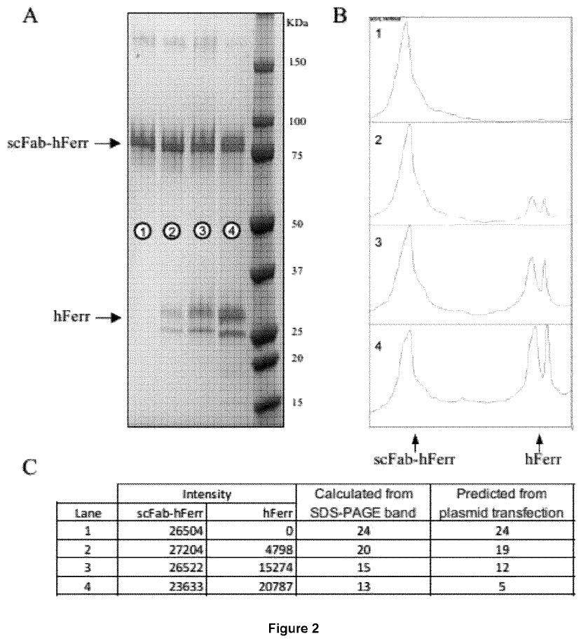

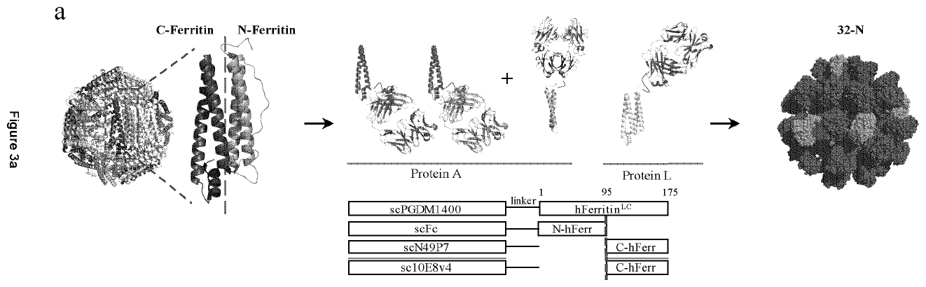

SDS-PAGE bands corresponding to scFab-apoferritin and unconjugated apoferritin were quantified by densitometry using the ImageJ software (rsb.info.nih.gov/iil/) (FIG. 2a). Intensity plots of the bands in each lane (yellow box) are shown in FIG. 2b. The approximate number of scFabs displayed on the particles was calculated as follows: intensity of scFab band/total intensity, and compared with the theoretical numbers inferred from DNA ratios used for co-transfection (FIG. 2c).

### Multabodies Potently and Broadly Neutralize HIV-1

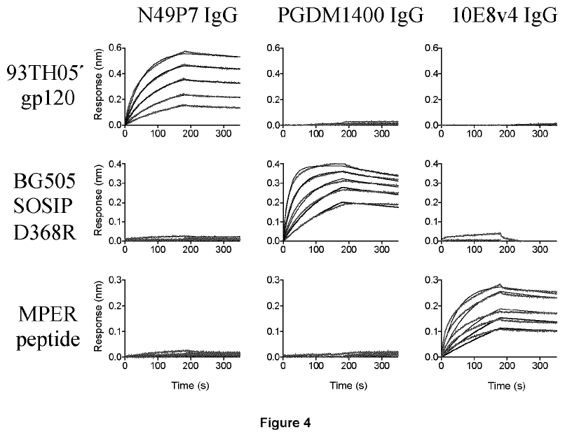

In view of these results, we sought to increase the coverage of PGDM1400 using our previously described Multabody platform based on an apoferritin split design (49). The strategy consists on the separation of the four-helix apoferritin subunit into two halves (N-ferritin and C-ferritin) and their N-terminal fusion to scFabs of different specificities (FIG. 3a). This approach allows inclusion of a higher number of Fabs on the surface of the nanocage resulting in a final molecule with higher avidity. In addition, the design allows the efficient combination of three different antibody specificities as well as a fragment crystallizable (Fc) to endow the molecule with IgG-like properties, such as ease of purification leveraging Protein A affinity. Specifically, we combined scFab PGDM1400, with scFabs of the near-pan neutralizing antibodies 10E8v4 (a modified 10E8 with improved solubility(51)) and N49P7 and the Fc fragment of the human IgG1 isotype (FIG. 3a). As expected, the resulting Multabody, termed 32-N formed highly-decorated and homogeneous particles (FIGS. 3b and c) with biophysical and functional properties similar to the corresponding IgGs (FIG. 3d). Epitope engagement by the tri-specific Multabodies was assessed in binding kinetics experiments using epitope-specific molecules: BG505 SOSIP D368R (PGDM1400), 93TH057 gp120 (N49P7), MPER peptide (10E8v4) and the human Fc receptors (FcRn and FcγR) (Fc) (FIG. 3e). Binding to the three epitope-specific antigens with high apparent binding affinities and no detectable dissociation confirms the presence of all the specificities in the Multabody. No individual IgG molecules had the ability to bind all antigens (FIG. 4).

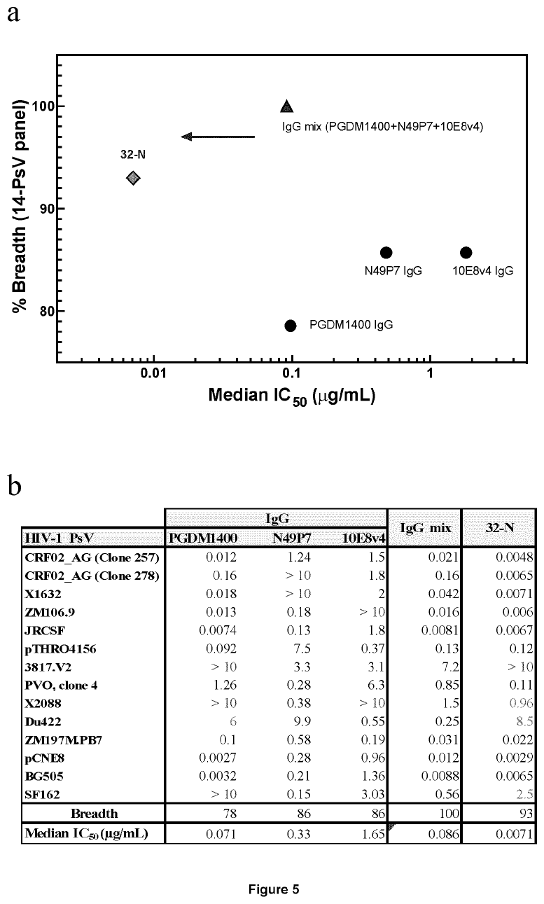

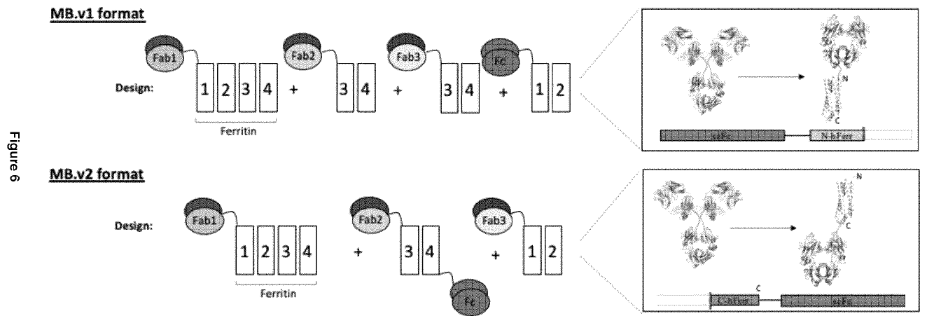

Neutralization potency and breadth of Multabody 32-N was assessed against a panel of 14-PsVs in a standardized in vitro TZM-bl neutralization assays(50). The 14-PsV panel was designed to include low-sensitivity PsVs with a minimum of one resistant PsV for each bNAb being evaluated (cutoff IC₅₀ set at 10 μg/mL). The IC₅₀ value and breadth of the Multabodies were compared to each individual IgG and an IgG cocktail that contains the same relative amount of IgG present in the Multabody. 32-N MB had a median IC₅₀ value of 0.0071 μg/mL (3 pM) (FIG. 5). However, despite the 12-fold improved potency achieved with respect a mixture of the parental IgGs, the Multabody was not able to reach 100% neutralization against this panel (93% breadth with a cutoff limit of 10 μg/mL). In addition, inspection of the individual IC₅₀ values revealed that the Multabody displays low potency against those PsVs resistant to PGDM1400 neutralization (FIG. 5b). This data suggests that the neutralization properties of the Multabody heavily depend on one out of the three antibody specificities within the particle, in this case PGDM1400.

### Engineering the Apoferritin Scaffold

To further improve the neutralization properties of the Multabody, we introduced some modifications to its design and made a second-generation versions (MB.v2 and MB.v3). In the original Multabody (hereinafter referred to as MB.v1), the scFc is located at the N terminus of the N-ferritin half, and only one Fab, either Fab2 or Fab3, is incorporated in the Multabody per each functional Fc homodimer (FIG. 6 top row).

In comparison, in the optimized MB.v2 the Fc fragment is positioned to the C-terminus of the C-ferritin half. Driven by split apoferritin complementation, two different Fabs (Fab2 and Fab3) and one Fc domain self-associate in a 1:1:1 ratio and in a defined position relative to each other (FIG. 6; bottom row; see “MB.v2 format”).

In the case of MB.v3, this optimized version contains a higher number of Fabs per Fc homodimer. In this design, two Fab2 and two Fab3 are incorporated into the Multabody per dimeric Fc. To attain this, a monomeric Fc fragment (i.e. one Fc chain) and a scFab are positioned at the C terminus and the N terminus of the C-ferritin half, respectively (FIG. 7a). As a result, dimerization of a functional Fc homodimer drives assembly of the MB.v3 particle together with split ferritin complementation and ferritin subunit oligomerization (FIG. 7b).

Importantly, these designs ensures assembly of higher number of Fabs different from PGDM1400 (i.e. Fab2 and Fab3), thus favoring a more balanced avidity for each of the three Fabs in the fully-assembled Multabody.

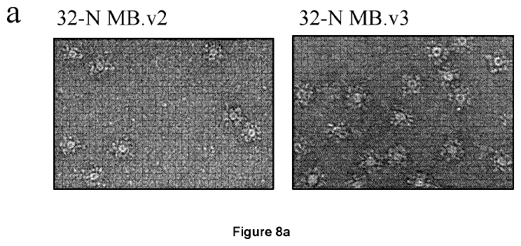

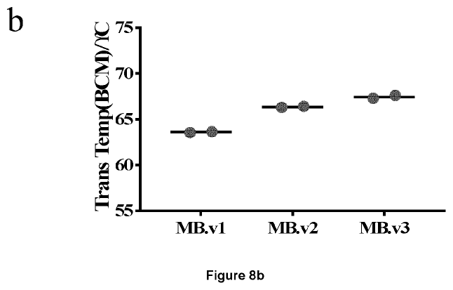

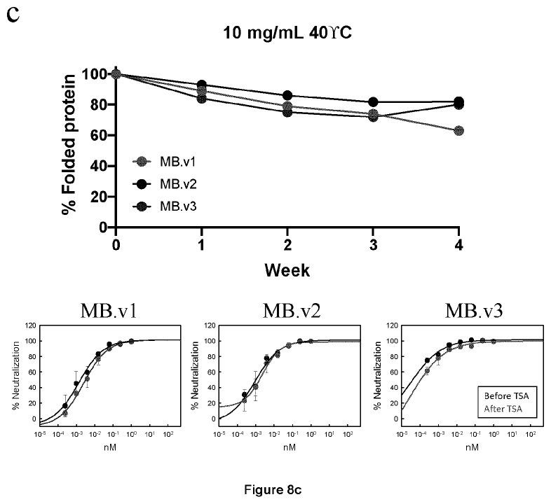

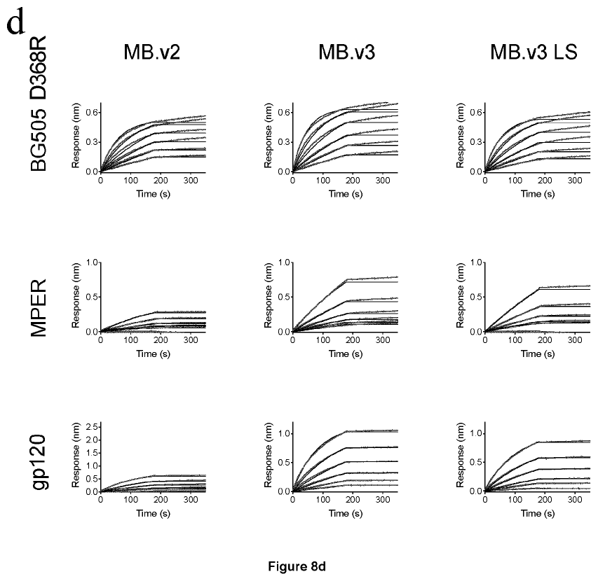

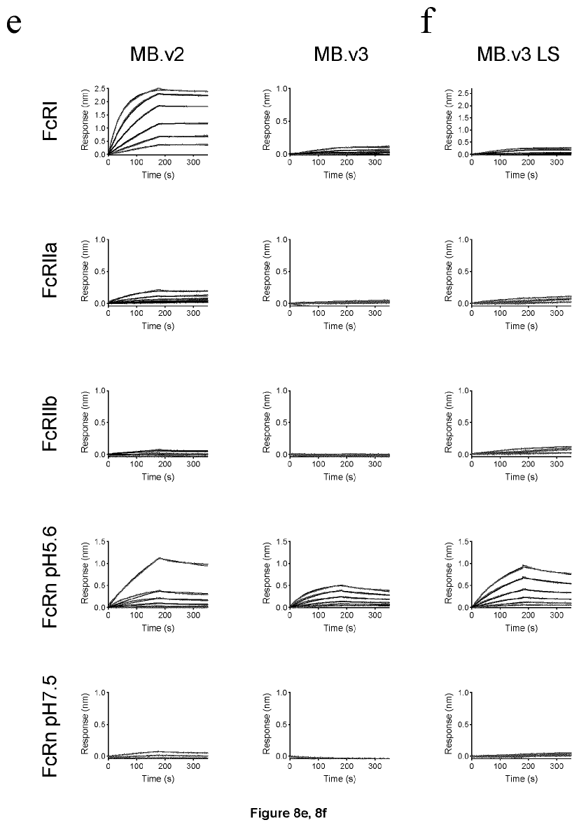

The resulting Multabody particles (MB.v2 and MB.v3) assembled into highly stable and well-formed spherical particles with no significant differences in the morphology and biophysical properties between the different Multabody versions (FIG. 8a-c). Antigen (FIG. 8d) and Fc receptor binding (FIG. 8e) confirmed the proper folding of the Multabodies including proper dimerization of the Fc fragment in the case of MB.v3. In the MB.v2 and MB.v3 formats, the positioning of the Fc at the C terminus of the ferritin leads to the formation of particles with inverted and more distantly located Fc domains and, consequently, reduced Fc avidity. Hence, binding of these Multabodies to FcγRI and FcRn at acidic pH was reduced in comparison to 32-N MB.v1 (FIG. 3e and FIG. 8e) and binding to FcRn at acidic pH yielded measurable K_{off} even in the presence of the half-life extension mutations LS (M428L/N434S) as shown for the 32-N MB.v3 LS variant (FIG. 8f).

### Extraordinary Potency and Pan-Neutralization Breadth Achieved by Second Generation Multabody Particles

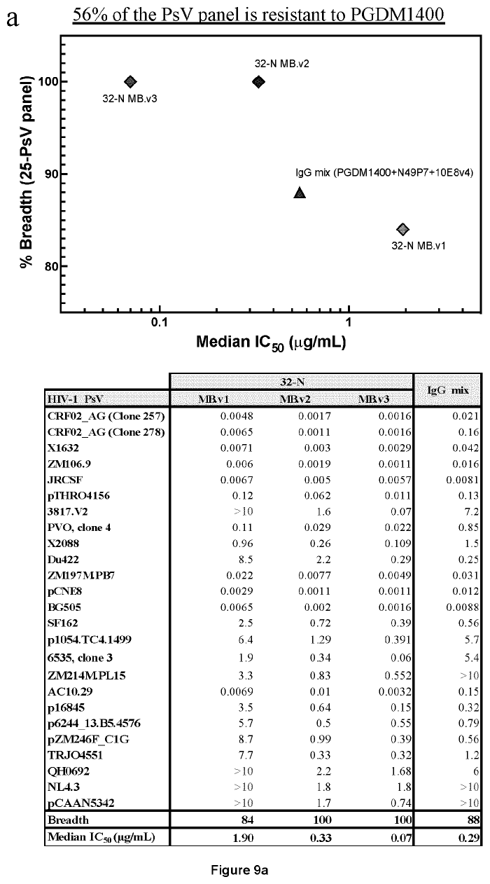

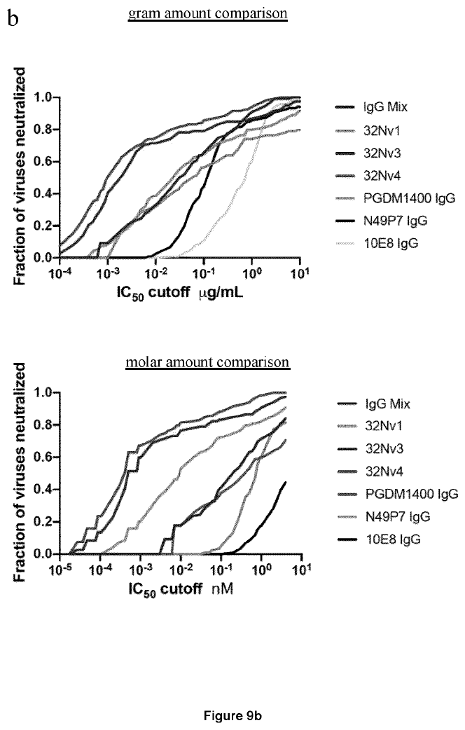

Next, we assessed the neutralization potency of the optimized Multabody versions (MB.v2 and MB.v3) against a PsV panel generated through addition of 11 HIV-1 strains highly resistant to PGDM1400 to our previous panel. The resulting 25-PsV panel contains 56% of PsV variants resistant (cutoff IC₅₀ set at 10 μg/mL) to PGDM1400 neutralization (FIG. 9a). As expected, breadth and potency of the 32-N MB.v1 was greatly affected in the presence of PGDM1400 resistant PsVs (FIG. 9a). However, as engineered, the neutralization profile of antibodies N49P7 and 10E8v4 were more dominant in 32-N MB.v2 and 32-N MB.v3 allowing this optimized Multabodies to achieve pan-neutralization while preserving the enhanced neutralization potency previously observed for this type of molecule (FIG. 9a). When tested against an extended multiclade panel of 118 PsV, 32-N MB.v3 matched the pan-neutralization breadth of the corresponding IgG cocktail (100% virus coverage, cutoff IC₅₀ set at 10 μg/mL), yet displayed a remarkable neutralization potency (FIG. 9b). Specifically, the IgG cocktail and 32-N MB.v1 were only able to neutralize 9% and 8% of the PsV with an IC₅₀ value of 0.001 μg/mL, respectively, while in the case of 32-N MB.v3, 50% of the PsVs were still neutralized with an IC₅₀ value of only 0.001 μg/mL (FIG. 9b). Remarkably, 32-N MB.v3 achieved a median IC₅₀ value of only 0.0009 μg/mL (0.4 pM) and hence achieved pan-neutralization 32- and 490-fold more potently in mass and molarity, respectively, compared to the IgG cocktail (FIG. 9b).

### In Vivo Pharmacokinetics of Multabodies are Similar to Corresponding IgG

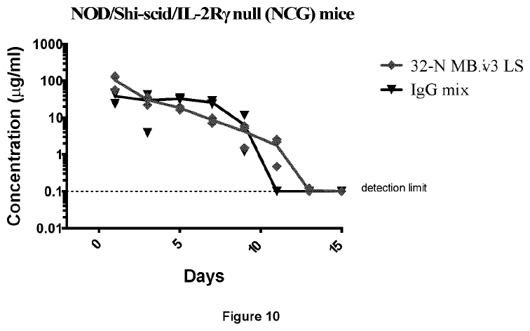

We next examined the in vivo bioavailability of 32-N MB.v3 containing the IgG1 Fc fragment with the LS mutation. A single dose of 5 mg/kg was administered subcutaneously in NOD/Shi-scid/lL-2Rγnull immunodeficient mouse strain (NCG) and the level of Multabody in the sera was detected every two days for 15 consecutive days. Multabody administration was well tolerated with no decrease in body weight or visible signs of toxicity. In addition, the Multabody showed days of in vivo exposure with a similar rate of decay as the parental IgG cocktail (FIG. 10). This data demonstrates the feasibility of the Multabody platform to generate molecules with bioavailability properties and provide an encouraging set of initial in vivo validation for its developability.

### REFERENCES

1. Conley, A. J. et al. Neutralization of divergent human immunodeficiency virus type 1 variants and primary isolates by IAM-41-2F5, an anti-gp41 human monoclonal antibody. Proc. Natl. Acad. Sci. (2006) doi:10.1073/pnas.91.8.3348.
2. Stiegler, G. et al. A Potent Cross-Clade Neutralizing Human Monoclonal Antibody against a Novel Epitope on gp41 of Human Immunodeficiency Virus Type 1. AIDS Res. Hum. Retroviruses (2002) doi:10.1089/08892220152741450.
3. Zwick, M. B. et al. Broadly Neutralizing Antibodies Targeted to the Membrane-Proximal External Region of Human Immunodeficiency Virus Type 1 Glycoprotein gp41. J. Virol. (2002) doi:10.1 128/jvi.75.22.10892-10905.2001.
4. BUCHACHER, A. et al. Generation of Human Monoclonal Antibodies against HIV-1 Proteins, Electrofusion and Epstein-Barr Virus Transformation for Peripheral Blood Lymphocyte Immortalization. AIDS Res. Hum. Retroviruses (2009) doi:10.1089/aid.1994.10.359.
5. Barbas, C. F. et al. Recombinant human Fab fragments neutralize human type 1 immunodeficiency virus in vitro. Proc. Natl. Acad. Sci. U.S.A. (1992).
6. Burton, D. R. et al. Efficient neutralization of primary isolates of HIV-1 by a recombinant human monoclonal antibody. Science (80-.). (1994) doi:10.1126/science.7973652.
7. Wu, X. et al. Rational design of envelope identifies broadly neutralizing human monoclonal antibodies to HIV-1. Science (80-.). (2010) doi:10.1126/science.1187659.
8. Scheid, J. F. et al. Broad diversity of neutralizing antibodies isolated from memory B cells in HIV-infected individuals. Nature (2009) doi:10.1038/nature07930.
9. Sok, D. et al. A Recombinant HIV Envelope Trimer Selects for Quaternary Dependent Antibodies Targeting the Trimer Apex. AIDS Res. Hum. Retroviruses (2014) doi:10.1089/aid.2014.5002.abstract.
10. Walker, L. M. et al. Broad and Potent Neutralizing Antibodies from an African Donor Reveal a New HIV-1 Vaccine Target TL-326. Science (80-.). (2009) doi:10.1 126/science. 1178746.
11. Walker, L. M. et al. Broad neutralization coverage of HIV by multiple highly potent antibodies. Nature (2011) doi:10.1038/nature10373.
12. Doria-Rose, N. A. et al. Developmental pathway for potent V1V2-directed HIV-neutralizing antibodies. Nature (2014) doi:10.1038/nature13036.
13. Huang, J. et al. Broad and potent neutralization of HIV-1 by a gp41-specific human antibody. Nature (2012) doi:10.1038/nature11544.
14. Sajadi, M. M. et al. Identification of Near-Pan-neutralizing Antibodies against HIV-1 by Deconvolution of Plasma Humoral Responses. Cell (2018) doi:10.1016/j.cell.2018.03.061.
15. Scheid, J. F. et al. Sequence and Structural Convergence of Broad and Potent HIV Antibodies That Mimic CD4 Binding. Science (80-.). (2011) doi:10.1126/science.1207227.
16. Huang, J. et al. Identification of a CD4-Binding-Site Antibody to HIV that Evolved Near-Pan Neutralization Breadth. Immunity (2016) doi:10.1016/j.immuni.2016.10.027.
17. Pejchal, R. et al. A potent and broad neutralizing antibody recognizes and penetrates the HIV glycan shield. Science (80-.). (2011) doi:10.1126/science.1213256.
18. Blattner, C. et al. Structural delineation of a quaternary, cleavage-dependent epitope at the gp41-gp120 interface on intact HIV-1 env trimers. Immunity (2014) doi:10.1016/j.immuni.2014.04.008.
19. Mouquet, H. et al. Complex-type N-glycan recognition by potent broadly neutralizing HIV antibodies. Proc. Natl. Acad. Sci. (2012) doi:10.1073/pnas.1217207109.
20. Baba, T. W. et al. Human neutralizing monoclonal antibodies of the IgG1 subtype protect against mucosal simian-human immunodeficiency virus infection. Nat. Med. (2000) doi:10.1038/72309.
21. Hessell, A. J. et al. Effective, low-titer antibody protection against low-dose repeated mucosal SHIV challenge in macaques. Nat. Med. (2009) doi:10.1038/nm.1974.
22. Hessell, A. J. et al. Broadly neutralizing human anti-HIV antibody 2G12 is effective in protection against mucosal SHIV challenge even at low serum neutralizing titers. PLoS Pathog. (2009) doi:10.1371/journal.ppat.1000433.
23. Hofmann-Lehmann, R. et al. Postnatal pre- and postexposure passive immunization strategies: Protection of neonatal macaques against oral simian-human immunodeficiency virus challenge. J. Med. Primatol. (2002) doi:10.1034/j.1600-0684.2002.01014.x.
24. Hofmann-Lehmann, R. et al. Postnatal Passive Immunization of Neonatal Macaques with a Triple Combination of Human Monoclonal Antibodies against Oral Simian-Human Immunodeficiency Virus Challenge. J. Virol. (2002) doi:10.1128/ijvi.75.16.7470-7480.2001.
25. van der Velden, Y. U. et al. Short Communication: Protective Efficacy of Broadly Neutralizing Antibody PGDM1400 Against HIV-1 Challenge in Humanized Mice. AIDS Res. Hum. Retroviruses (2018) doi:10.1089/aid.2018.0114.
26. Klein, F. et al. HIV therapy by a combination of broadly neutralizing antibodies in humanized mice. Nature (2012) doi:10.1038/nature11604.
27. Deruaz, M. et al. Protection of humanized mice from repeated intravaginal HIV challenge by passive immunization: A model for studying the efficacy of neutralizing antibodies in vivo. J. Infect. Dis. (2016) doi:10.1093/infdis/jiw203.
28. Horwitz, J. A. et al. HIV-1 suppression and durable control by combining single broadly neutralizing antibodies and antiretroviral drugs in humanized mice. Proc. Natl. Acad. Sci. (2013) doi:10.1073/pnas.1315295110.
29. Mehandru, S. et al. Adjunctive Passive Immunotherapy in Human Immunodeficiency Virus Type 1-Infected Individuals Treated with Antiviral Therapy during Acute and Early Infection. J. Virol. (2007) doi:10.1128/jvi.01340-07.
30. Caskey, M. et al. Viraemia suppressed in HIV-1-infected humans by broadly neutralizing antibody 3BNC117. Nature (2015) doi:10.1038/nature14411.
31. Caskey, M. et al. Antibody 10-1074 suppresses viremia in HIV-1-infected individuals. Nat. Med. (2017) doi:10.1038/nm.4268.
32. Lynch, R. M. et al. Virologic effects of broadly neutralizing antibody VRC01 administration during chronic HIV-1 infection. Sci. Transl. Med. (2015) doi:10.1126/scitranslmed.aad5752.
33. Ledgerwood, J. E. et al. Safety, pharmacokinetics and neutralization of the broadly neutralizing HIV-1 human monoclonal antibody VRC01 in healthy adults. Clin. Exp. Immunol. (2015) doi:10.1111/cei.12692.
34. Toma, J. et al. Loss of Asparagine-Linked Glycosylation Sites in Variable Region 5 of Human Immunodeficiency Virus Type 1 Envelope Is Associated with Resistance to CD4 Antibody Ibalizumab. J. Virol. (2011) doi:10.1128/jvi.02237-10.
35. Jacobson, J. M. et al. Antiviral Activity of Single-Dose PRO 140, a CCR5 Monoclonal Antibody, in HIV-Infected Adults. J. Infect. Dis. (2008) doi:10.1086/592169.
36. Onafuwa-Nuga, A. & Telesnitsky, A. The Remarkable Frequency of Human Immunodeficiency Virus Type 1 Genetic Recombination. Microbiol. Mol. Biol. Rev. (2009) doi:10.1128/mmbr.00012-09.
37. Sather, D. N. et al. Broadly Neutralizing Antibodies Developed by an HIV-Positive Elite Neutralizer Exact a Replication Fitness Cost on the Contemporaneous Virus. J. Virol. (2012) doi:10.1128/JVI.01893-12.
38. Lynch, R. M. et al. HIV-1 Fitness Cost Associated with Escape from the VRC01 Class of CD4 Binding Site Neutralizing Antibodies. J. Virol. (2015) doi:10.1128/jvi.03608-14.
39. Pietzsch, J. et al. Human anti-HIV-neutralizing antibodies frequently target a conserved epitope essential for viral fitness. J. Exp. Med. (2010) doi:10.1084/jem.20101176.
40. Asokan, M. et al. Bispecific Antibodies Targeting Different Epitopes on the HIV-1 Envelope Exhibit Broad and Potent Neutralization. J. Virol. (2015) doi:10.1128/ijvi.02097-15.
41. Bournazos, S., Gazumyan, A., Seaman, M. S., Nussenzweig, M. C. & Ravetch, J. V. Bispecific Anti-HIV-1 Antibodies with Enhanced Breadth and Potency. Cell (2016) doi:10.1016/j.cell.2016.04.050.
42. Huang, Y. et al. Engineered Bispecific Antibodies with Exquisite HIV-1-Neutralizing Activity. Cell (2016) doi:10.1016/j.cell.2016.05.024.
43. Xu, L. et al. Trispecific broadly neutralizing HIV antibodies mediate potent SHIV protection in macaques. Science (80-.). (2017) doi:10.1126/science.aan8630.
44. Steinhardt, J. J. et al. Rational design of a trispecific antibody targeting the HIV-1 Env with elevated anti-viral activity. Nat. Commun. (2018) doi:10.1038/s41467-018-03335-4.
45. Khan, S. N. et al. Targeting the HIV-1 Spike and Coreceptor with Bi- and Trispecific Antibodies for Single-Component Broad Inhibition of Entry. J. Virol. (2018) doi:10.1 128/jvi.00384-18.
46. Rudicell, R. S. et al. Enhanced Potency of a Broadly Neutralizing HIV-1 Antibody In Vitro Improves Protection against Lentiviral Infection In Vivo. J. Virol. (2014) doi:10.1128/JVI.02213-14.
47. Kwon, Y. D. et al. Surface-Matrix Screening Identifies Semi-specific Interactions that Improve Potency of a Near Pan-reactive HIV-1-Neutralizing Antibody. Cell Rep. (2018) doi:10.1016/j.celrep.2018.01.023.
48. Rujas, E. et al. Functional Optimization of Broadly Neutralizing HIV-1 Antibody 10E8 by Promotion of Membrane Interactions. J. Virol. (2018) doi:10.1128/jvi.02249-17.
49. Diskin, R. et al. Increasing the potency and breadth of an HIV antibody by using structure-based rational design. Science (80-.). (2011) doi:10.1126/science.1213782.
50. Rujas, E. et al. Multivalency transforms SARS-CoV-2 antibodies into broad and ultrapotent neutralizers. bioRxiv (2020).
51. Kwon, Y. D. et al. Optimization of the Solubility of HIV-1-Neutralizing Antibody 10E8 through Somatic Variation and Structure-Based Design. J. Virol. (2016) doi:10.1128/jvi.03246-15.
52. Montefiori, D. C. Measuring HIV neutralization in a luciferase reporter gene assay. Methods Mol. Biol. (2009) doi:10.1007/978-1-59745-170-3_26.

What is claimed is:

1. A fusion protein comprising a first nanocage monomer or a subunit thereof linked to an Fc monomer, wherein a plurality of the fusion proteins are capable of self-assembling to form a nanocage comprising one or more Fc dimers.

2. The fusion protein of claim 1, wherein the Fc monomer is linked to the first nanocage monomer or subunit thereof at the N- or C-terminus of the first nanocage monomer or subunit thereof, preferably at the C-terminus.

3. The fusion protein of claim 1 or 2, wherein the subunit comprises an N-subunit or a C-subunit, corresponding substantially to the N-terminal half of a nanocage monomer and the C-terminal half of a nanocage monomer, respectively, wherein the N-subunit and the C-subunit are capable of self-assembling to form a nanocage monomer.

4. The fusion protein of claim 3, wherein the Fc monomer is linked to the N-subunit or C-subunit at the C-terminus of the N-subunit or C-subunit, preferably wherein the Fc monomer is linked to the C-subunit at the C-terminus.

5. The fusion protein of any one of claims 1 to 4, wherein the first nanocage monomer or subunit thereof is further linked to a first bioactive moiety.

6. The fusion protein of claim 5, wherein the first bioactive moiety is linked to the first nanocage monomer or subunit thereof at the N- or C-terminus of the first nanocage monomer or subunit thereof, preferably the N-terminus.

7. The fusion protein of claim 6, wherein the first bioactive moiety is linked to the N-subunit or C-subunit at the N-terminus of the N-subunit or C-subunit, preferably wherein the first bioactive moiety is linked to the C-subunit at the N-terminus.

8. The fusion protein of any one of claims 5 to 7, wherein the first bioactive moiety decorates the interior and/or exterior surface, preferably the exterior surface, of the assembled nanocage.

9. The fusion protein of any one of claims 5 to 8, wherein the first bioactive moiety comprises a first antigen-binding moiety.

10. The fusion protein of claim 9, wherein the first antigen-binding moiety comprises an antibody or fragment thereof.

11. The fusion protein of claim 10, wherein the first antigen-binding moiety comprises a Fab fragment.

12. The fusion protein of claim 10 or 11, wherein the antibody or fragment thereof comprises a scFab fragment, a scFv fragment, a sdAb fragment, a VHH domain or a combination thereof.

13. The fusion protein of claim 10, wherein the antibody or fragment thereof comprises a heavy and/or light chain of a Fab fragment.

14. The fusion protein of any one of claims 5 to 13, wherein the first antigen-binding moiety binds specifically to an antigen associated with an antibody-preventable and/or antibody-treatable condition.

15. The fusion protein of claim 14, wherein the antigen is associated with an infectious agent, including a virus, bacteria, a parasite, a fungus, or a yeast, a cancer, or an immune disease, including an autoimmune disease.

16. The fusion protein of claim 15, wherein the first antigen-binding moiety comprises an HIV-1 specific antigen-binding moiety.

17. The fusion protein of claim 16, wherein the HIV-1-specific antigen-binding moiety binds to BG505 SOSIP_D368R, 93TH057 gp120, and/or an MPER peptide.

18. The fusion protein of claim 17, wherein the HIV-1 specific antigen-binding moiety comprises an HIV-1 specific antigen-binding moiety from PGDM1400, 10E8v4, and/or N49P7.

19. The fusion protein of any one of claims 1 to 18, wherein the fusion protein comprises the first nanocage monomer or subunit thereof linked to the Fc monomer at the C-terminus of the first nanocage monomer or subunit thereof and linked to the first bioactive moiety at the N-terminus of the first nanocage monomer or subunit thereof.

20. The fusion protein of any one of claims 5 to 19, wherein the fusion protein comprises the C-subunit linked to the Fc monomer at the C-terminus of the C-subunit and linked to the first bioactive moiety at the N-terminus of the C-subunit.

21. The fusion protein of claim 20, in combination with an N-subunit or with a fusion protein comprising an N-subunit.

22. The fusion protein of claim 21, wherein the N-subunit is linked to a second bioactive moiety at the N- or C-terminus, preferably the N-terminus.

23. The fusion protein of claim 22, wherein the second bioactive moiety comprises a second antigen-binding moiety, and wherein, if the first bioactive moiety comprises the first antigen-binding moiety, the second antigen-binding moiety may be the same or different from the first antigen-binding moiety.

24. The fusion protein of claim 23, wherein the second antigen-binding moiety comprises an antibody or fragment thereof.

25. The fusion protein of claim 24, wherein the second antigen-binding moiety comprises a Fab fragment.

26. The fusion protein of claim 24, wherein the antibody or fragment thereof comprises a scFab fragment, a scFv fragment, a sdAb fragment, a VHH domain or a combination thereof.

27. The fusion protein of claim 24, wherein the antibody or fragment thereof comprises a heavy and/or light chain of a Fab fragment.

28. The fusion protein of any one of claims 21 to 27, wherein the N-subunit is further linked to an Fc monomer at the N- or C-terminus of the N-subunit, preferably the C-terminus.

29. The fusion protein of any one of claims 1 to 28, wherein the Fc monomer is derived from an IgG, IgA, IgD, IgM, or IgE, and is preferably human.

30. The fusion protein of claim 29, wherein the Fc monomer is derived from an IgG, such as IgG1, IgG2, IgG3, or IgG4.

31. The fusion protein of claim 30, wherein the Fc monomer is an IgG1 Fc monomer.

32. The fusion protein any one of claims 1 to 31, wherein the Fc monomer comprises one or more mutations or sets of mutations that modulate the half-life of the fusion protein from, for example, minutes or hours to several days, weeks, or months.

33. The fusion protein of claim 32, wherein the Fc monomer comprises a mutation at one or more of L234, L235, G236, G237, M252, I253, S254, T256, P329, A330, M428, N434, or a combination thereof (wherein numbering is according to the EU index), such as M428L and N434S (“LS”); M252Y, S254T and T256E (“YTE”); L234A and L235A (“LALA”); I253A; L234A, L235A, and P329G (“LALAP”); G236R; G237A; and/or A330L or a combination thereof.

34. The fusion protein of any one of claims 1 to 33, wherein from about 3 to about 100 nanocage monomers, such as 24, 32, 48, or 60 nanocage monomers, or from about 4 to about 200 nanocage monomer subunits, such as 4, 6, 8, 10, 12, 14, 18, 20, 22, 24, 26, 28, 30, 32, 34, 36, 38, 40, 42, 44, 46, 48, 50, or more, optionally in combination with one or more whole nanocage monomers, are capable of self-assembling to form a nanocage.

35. The fusion protein of any one of claims 1 to 34, wherein the first nanocage monomer or subunit thereof is selected from ferritin, apoferritin, encapsulin, SOR, lumazine synthase, pyruvate dehydrogenase, carboxysome, vault proteins, GroEL, heat shock protein, E2P, MS2 coat protein, fragments thereof, and variants thereof.

36. The fusion protein of claim 35, wherein the first nanocage monomer or subunit thereof is apoferritin, optionally human apoferritin.

37. The fusion protein of claim 36, wherein the first nanocage monomer or subunit thereof is an apoferritin light chain, optionally human apoferritin light chain.

38. The fusion protein of claim 36 or 37, wherein the fusion protein comprises a first apoferritin subunit, optionally a first human apoferritin subunit, and wherein the first apoferritin subunit is capable of self-assembling with a second apoferritin subunit.

39. The fusion protein of claim 38, wherein the first and second apoferritin monomer subunits interchangeably comprise the “N” and “C” regions of apoferritin.

40. The fusion protein of claim 39, wherein the “N” region of apoferritin comprises or consists of a sequence at least 70% (such as at least 75%, 80%, 85%, 90%, 95%, 96%, 97%, 98%, 99%, or 100%) identical to:
(SEQ ID NO: 1)
MSSQIRQNYSTDVEAAVNSLVNLYLQASYTYLSLGFYFDRDDVALEGVS
HFFRELAEEKREGYERLLKMQNQRGGRALFQDIKKPAEDEW
or
(SEQ ID NO: 15)
SSQIRQNYSTDVEAAVNSLVNLYLQASYTYLSLGFYFDRDDVALEGVSH
FFRELAEEKREGYERLLKMQNQRGGRALFQDIKKPAEDEW.

41. The fusion protein of claim 39 or 40, wherein the “C” region of apoferritin comprises or consists of a sequence at least 70% (such as at least 75%, 80%, 85%, 90%, 95%, 96%, 97%, 98%, 99%, or 100%) identical to:
(SEQ ID NO: 2)
GKTPDAMKAAMALEKKLNQALLDLHALGSARTDPHLCDFLETHFLDEEV
KLIKKMGDHLTNLHRLGGPEAGLGEYLFERLTLRHD
or
(SEQ ID NO: 16)
GKTPDAMKAAMALEKKLNQALLDLHALGSARTDPHLCDFLETHFLDEEV
KLIKKMGDHLTNLHRLGGPEAGLGEYLFERLTLKHD.

42. The fusion protein of any one of claims 1 to 41, wherein the Fc monomer is linked to the first nanocage monomer or subunit thereof through a linker.

43. The fusion protein of any one of claims 5-42, wherein the first bioactive moiety is linked to the first nanocage monomer or subunit thereof through a linker.

44. The fusion protein of any of claims 22-43, wherein the second bioactive moiety is linked to the N-subunit through a linker.

45. The fusion protein of any one of claims 42-44, wherein the linker is flexible or rigid and comprises from about 1 to about 100 amino acid residues, such as from about 1 to about 70 amino acid residues, such as from about 1 to about 30 amino acid residues, such as from about 8 to about 16 amino acid residues.

46. The fusion protein of any one of claims 42 to 45, wherein the linker comprises a GS domain.

47. The fusion protein of claim 46, wherein the GS domain comprises a GS repeat, a GGS repeat, a GGGS (SEQ ID NO:11) repeat, and/or a GGGGS (SEQ ID NO:12) repeat, such as 1, 2, 3, 4, or more GGGGS (SEQ ID NO:12) repeats.

48. The fusion protein of claim 47, wherein the linker comprises or consists of a sequence at least 70% (such as at least 75%, 80%, 85%, 90%, 95%, 96%, 97%, 98%, 99%, or 100%) identical to:
(SEQ ID NO: 4)
GGGGSGGGGSGGGGSGGGGSGGGGSGG.

49. The fusion protein of claim 47, wherein the linker comprises or consists of a sequence at least 70% (such as at least 75%, 80%, 85%, 90%, 95%, 96%, 97%, 98%, 99%, or 100%) identical to:
(SEQ ID NO: 5)
GGSGGSGGSGGSGGGSGGSGGSGGSG.

50. A nanocage comprising at least one fusion protein of any one of claims 1 to 49 and at least one second nanocage monomer or subunit thereof that self-assembles with the fusion protein.

51. The nanocage of claim 50, wherein the fusion protein comprises a first nanocage monomer subunit, the second nanocage monomer or subunit thereof is a second nanocage monomer subunit, and the second nanocage monomer subunit self-assembles with the fusion protein to form the nanocage monomer.

52. The nanocage of claim 50 or 51, wherein from about 1% to about 100%, such as from about 1%, 4%, 8%, 10%, 12%, 15%, 20%, 25%, 30%, 35%, 40%, 45%, 50%, 55%, 60%, 65%, 70%, 75%, 80%, 85%, 90%, or 95%, to about 4%, 8%, 10%, 12%, 15%, 20%, 25%, 30%, 35%, 40%, 45%, 50%, 55%, 60%, 65%, 70%, 75%, 80%, 85%, 90%, 95%, or 100%, such as from about 20% to about 80%, of the nanocage monomers or subunits thereof are comprised within a fusion protein of any one of claims 1 to 39.

53. The nanocage of claim 52, wherein each nanocage monomer or subunit thereof is comprised within a fusion protein of any one of claims 1 to 49.

54. The nanocage of any one of claims 50 to 53, comprising 1 bioactive moiety or at least 2, 3, 4, 5, 6, 7, 8, 9, or 10 different bioactive moieties, such as 2 or 3 different bioactive moieties.

55. The nanocage of any one of claims 50 to 54, wherein the nanocage is multivalent.

56. The nanocage of any one of claims 50 to 55, wherein the nanocage is multispecific.

57. The nanocage of any one of claims 50 to 56, wherein at least one bioactive moiety decorates the exterior surface of the nanocage and at least one Fc dimer decorates the exterior surface of the nanocage.

58. The nanocage of claim 57, wherein at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 bioactive moieties decorate the exterior surface of the nanocage and at least two Fc dimers decorate the exterior surface of the nanocage.

59. The nanocage of any one of claims 50 to 58, comprising:
(a) at least one nanocage monomer fused to a first antigen-binding moiety, such as a Fab,
(b) at least one N-subunit fused to a second antigen-binding moiety, such as a Fab, and
(c) and at least one C-subunit fused to:
(i) a third antigen-binding moiety, such as a Fab at one terminus, and
(ii) a Fc monomer at the other terminus.

60. The nanocage of claim 59, wherein each antigen-binding moiety is linked to the N-terminus of the nanocage monomer or subunit thereof and wherein the Fc monomer is linked to the C-terminus of the C-subunit.

61. The nanocage of any one of claims 50 to 60, wherein the antigen-binding moiety binds specifically to an antigen associated with an antibody-preventable and/or antibody-treatable condition.

62. The nanocage of claim 61, wherein the antigen is associated with an infectious agent, including a virus, bacteria, a parasite, a fungus, or a yeast, a cancer, or an immune disease, including an autoimmune disease.

63. The nanocage of claim 62, wherein each antigen-binding moiety is a different HIV-1-specific Fab.

64. The nanocage of claim 63, wherein the HIV-1-specific Fab binds to BG505 SOSIP_D368R, 93TH057 gp120, and/or an MPER peptide.

65. The nanocage of claim 64, wherein the HIV-1 specific Fab comprises PGDM1400 Fab, 10E8v4 Fab, and/or N49P7 Fab.

66. The nanocage of claim 65, comprising PGDM1400 Fab, 10E8v4 Fab, and N49P7 Fab.

67. The nanocage of any one of claims 50 to 66, comprising at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 antigen-binding moieties.

68. The nanocage of any one of claims 50 to 67, comprising at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 Fc monomers.

69. The nanocage of any one of claims 50 to 68, carrying a cargo molecule, such as a pharmaceutical agent, a diagnostic agent, and/or an imaging agent.

70. The nanocage of claim 69, wherein the cargo molecule is not fused to the fusion protein and is contained in the nanocage internally or wherein the cargo molecule is linked to the fusion protein or bound to the nanocage either internally or externally.

71. The nanocage of claim 69, wherein the cargo molecule is a protein and is fused to the fusion protein such that the cargo molecule is contained in the nanocage internally.

72. The nanocage of any one of claims 69 to 71, wherein the cargo molecule comprises a fluorescent protein, such as GFP, EGFP, Ametrine, and/or a flavin-based fluorescent protein, such as a LOV-protein, such as iLOV.

73. The nanocage of any one of claims 50 to 72, wherein the nanocage exhibits pan-virus neutralization breadth.

74. The nanocage of any one of claims 50 to 73, wherein the nanocage exhibits an average median IC₅₀ value against a multiclade panel of 118 pseudoviruses (PsV) of less than about 0.1 ug/mL, such as less than about 0.01 ug/mL, such as less than about 0.001 ug/mL.

75. The nanocage of any one of claims 50 to 74, wherein the nanocage exhibits an average median IC₅₀ value against a multiclade panel of 118 pseudoviruses (PsV) of less than about 42 pM, such as less than about 4.2 pM, such as less than about 0.42 pM.

76. The nanocage of any one of claims 50 to 75, wherein the nanocage exhibits an average median IC₅₀ value against a multiclade panel of 118 pseudoviruses (PsV) that is at least about 10, at least about 100, at least about 1000, at least about 10,000, or at least about 100,000 more potent than a cocktail of the corresponding bNAbs on a mass and/or molar basis.

77. A fusion protein comprising a first nanocage monomer or a subunit thereof linked to an scFc fragment at the C-terminus of the first nanocage monomer or subunit thereof, wherein a plurality of the fusion proteins are capable of self-assembling to form a nanocage.

78. The fusion protein of claim 77, wherein the subunit comprises an N-subunit or a C-subunit, corresponding substantially to the N-terminal half of a nanocage monomer and the C-terminal half of a nanocage monomer, respectively, wherein the N-subunit and the C-subunit are capable of self-assembling to form a nanocage monomer.

79. The fusion protein of claim 78, wherein the scFc fragment is linked to the N-subunit or C-subunit at the C-terminus of the N-subunit or C-subunit.

80. The fusion protein of any one of claims 77 to 79, wherein the first nanocage monomer or subunit thereof is further linked to a first bioactive moiety.

81. The fusion protein of claim 80, wherein the first bioactive moiety is linked to the first nanocage monomer or subunit thereof at the N- or C-terminus of the first nanocage monomer or subunit thereof, preferably the N-terminus.

82. The fusion protein of claim 81, wherein the first bioactive moiety is linked to the N-subunit or C-subunit at the N-terminus of the N-subunit or C-subunit, preferably wherein the first bioactive moiety is linked to the C-subunit at the N-terminus.

83. The fusion protein of any one of claims 80 to 82, wherein the first bioactive moiety decorates the interior and/or exterior surface, preferably the exterior surface, of the assembled nanocage.

84. The fusion protein of any one of claims 80 to 83, wherein the first bioactive moiety comprises a first antigen-binding moiety.

85. The fusion protein of claim 84, wherein the first antigen-binding moiety comprises an antibody or fragment thereof.

86. The fusion protein of claim 85, wherein the first antigen-binding moiety comprises a Fab fragment.

87. The fusion protein of claim 85 or 86, wherein the antibody or fragment thereof comprises a scFab fragment, a scFv fragment, a sdAb fragment, a VHH domain or a combination thereof.

88. The fusion protein of claim 85, wherein the antibody or fragment thereof comprises a heavy and/or light chain of a Fab fragment.

89. The fusion protein of any one of claims 77 to 88, wherein the first antigen-binding moiety binds specifically to an antigen associated with an antibody-preventable and/or antibody-treatable condition.

90. The fusion protein of claim 89, wherein the antigen is associated with an infectious agent, including a virus, bacteria, a parasite, a fungus, or a yeast, a cancer, or an immune disease, including an autoimmune disease.

91. The fusion protein of claim 90, wherein the first antigen-binding moiety comprises an HIV-1 specific antigen-binding moiety.

92. The fusion protein of claim 91, wherein the HIV-1 specific antigen-binding moiety binds to BG505 SOSIP_D368R, 93TH057 gp120, and/or an MPER peptide.

93. The fusion protein of claim 92, wherein the HIV-1 specific antigen-binding moiety comprises an HIV-1 specific antigen-binding moiety from PGDM1400, 10E8v4, and/or N49P7.

94. The fusion protein of any one of claims 77 to 93, wherein the fusion protein comprises the first nanocage monomer or a subunit thereof linked to the scFc fragment at the C-terminus of the first nanocage monomer or a subunit thereof and linked to a first bioactive moiety at the N-terminus of the nanocage monomer or a subunit thereof.

95. The fusion protein of any one of claims 79 to 94, wherein the fusion protein comprises the C-subunit linked to the scFc fragment at the C-terminus of the C-subunit and linked to a first bioactive moiety at the N-terminus of the C-subunit.

96. The fusion protein of claim 95, in combination with an N-subunit or a fusion protein comprising an N-subunit.

97. The fusion protein of claim 96, wherein the N-subunit is linked to a second bioactive moiety at the N- or C-terminus, preferably the N-terminus.

98. The fusion protein of claim 97, wherein the second bioactive moiety comprises a second antigen-binding moiety, and wherein, if the first bioactive moiety comprises the first antigen-binding moiety, the second antigen-binding moiety may be the same or different from the antigen-binding moiety that is linked to the C-subunit.

99. The fusion protein of claim 98, wherein the second antigen-binding moiety comprises an antibody or fragment thereof.

100. The fusion protein of claim 99, wherein the second antigen-binding moiety comprises a Fab fragment.

101. The fusion protein of claim 99 or 100, wherein the antibody or fragment thereof comprises a scFab fragment, a scFv fragment, a sdAb fragment, a VHH domain or a combination thereof.

102. The fusion protein of claim 99, wherein the antibody or fragment thereof comprises a heavy and/or light chain of a Fab fragment.

103. The fusion protein of any one of claims 96 to 102, wherein the N-subunit is further linked to a second scFc fragment at the N- or C-terminus of the N-subunit, preferably the C-terminus.

104. The fusion protein of any one of claims 77 to 103, wherein the scFc fragment is derived from an IgG, IgA, IgD, IgM, or IgE, and is preferably human.

105. The fusion protein of claim 104, wherein the scFc fragment is derived from an IgG, such as IgG1, IgG2, IgG3, or IgG4.

106. The fusion protein of claim 105, wherein the scFc fragment is an IgG1 scFc fragment.

107. The fusion protein any one of claims 77 to 106, wherein the scFc fragment comprises one or more mutations or sets of mutations that modulate the half-life of the fusion protein from, for example, minutes or hours to several days, weeks, or months.

108. The fusion protein of claim 107, wherein the scFc fragment comprises a mutation at one or more of L234, L235, G236, G237, M252, I253, S254, T256, P329, A330, M428, N434, or a combination thereof (wherein numbering is according to the EU index), such as M428L and N434S (“LS”); M252Y, S254T and T256E (“YTE”); L234A and L235A (“LALA”); I253A; L234A, L235A, and P329G (“LALAP”); G236R; G237A; and/or A330L or a combination thereof.

109. The fusion protein of any one of claims 77 to 108, wherein from about 3 to about 100 nanocage monomers, such as 24, 32, 48, or 60 nanocage monomers, or from about 4 to about 200 nanocage monomer subunits, such as 4, 6, 8, 10, 12, 14, 18, 20, 22, 24, 26, 28, 30, 32, 34, 36, 38, 40, 42, 44, 46, 48, 50, or more, optionally in combination with one or more whole nanocage monomers, are capable of self-assembling to form a nanocage.

110. The fusion protein of any one of claims 77 to 109, wherein the first nanocage monomer or subunit thereof is selected from ferritin, apoferritin, encapsulin, SOR, lumazine synthase, pyruvate dehydrogenase, carboxysome, vault proteins, GroEL, heat shock protein, E2P, MS2 coat protein, fragments thereof, and variants thereof.

111. The fusion protein of claim 110, wherein the first nanocage monomer or subunit thereof is apoferritin, optionally human apoferritin.

112. The fusion protein of claim 111, wherein the first nanocage monomer or subunit thereof is an apoferritin light chain, optionally human apoferritin light chain.

113. The fusion protein of claim 111 or 112, wherein the fusion protein comprises a first apoferritin subunit, optionally a first human apoferritin subunit, and wherein the first apoferritin subunit is capable of self-assembling with a second apoferritin subunit.

114. The fusion protein of claim 113, wherein the first and second apoferritin monomer subunits interchangeably comprise the “N” and “C” regions of apoferritin.

115. The fusion protein of claim 114, wherein the “N” region of apoferritin comprises or consists of a sequence at least 70% (such as at least 75%, 80%, 85%, 90%, 95%, 96%, 97%, 98%, 99%, or 100%) identical to:
(SEQ ID NO: 1)
MSSQIRQNYSTDVEAAVNSLVNLYLQASYTYLSLGFYFDRDDVALEGVS
HFFRELAEEKREGYERLLKMQNQRGGRALFQDIKKPAEDEW
or
(SEQ ID NO: 15)
SSQIRQNYSTDVEAAVNSLVNLYLQASYTYLSLGFYFDRDDVALEGVSH
FFRELAEEKREGYERLLKMQNQRGGRALFQDIKKPAEDEW.

116. The fusion protein of claim 114 or 115, wherein the “C” region of apoferritin comprises or consists of a sequence at least 70% (such as at least 75%, 80%, 85%, 90%, 95%, 96%, 97%, 98%, 99%, or 100%) identical to:
(SEQ ID NO: 2)
GKTPDAMKAAMALEKKLNQALLDLHALGSARTDPHLCDFLETHFLDEEV
KLIKKMGDHLTNLHRLGGPEAGLGEYLFERLTLRHD
or
(SEQ ID NO: 16)
GKTPDAMKAAMALEKKLNQALLDLHALGSARTDPHLCDFLETHFLDEEV
KLIKKMGDHLTNLHRLGGPEAGLGEYLFERLTLKHD.

117. The fusion protein of any one of claims 77 to 116, wherein the scFc fragment is linked to the first nanocage monomer or subunit thereof through a linker.

118. The fusion protein of any one of claims 80-117, wherein the first bioactive moiety is linked to the first nanocage monomer or subunit thereof through a linker.

119. The fusion protein of any one of claims 97-118, wherein the second bioactive moiety is linked to the N-subunit through a linker.

120. The fusion protein of any one of claims 117 to 119, wherein the linker is flexible or rigid and comprises from about 1 to about 100 amino acid residues, such as from about 1 to about 70 amino acid residues, such as from about 1 to about 30 amino acid residues, such as from about 8 to about 16 amino acid residues.

121. The fusion protein of any one of claims 117 to 120, wherein the linker comprises a GS domain.

122. The fusion protein of claim 121, wherein the GS domain comprises a GS repeat, a GGS repeat, a GGGS (SEQ ID NO:11) repeat, and/or a GGGGS (SEQ ID NO:12) repeat, such as 1, 2, 3, 4, or more GGGGS (SEQ ID NO:12) repeats.

123. The fusion protein of claim 122, wherein the linker comprises or consists of a sequence at least 70% (such as at least 75%, 80%, 85%, 90%, 95%, 96%, 97%, 98%, 99%, or 100%) identical to:
(SEQ ID NO: 4)
GGGGSGGGGSGGGGSGGGGSGGGGSGG.

124. The fusion protein of claim 122, wherein the linker comprises or consists of a sequence at least 70% (such as at least 75%, 80%, 85%, 90%, 95%, 96%, 97%, 98%, 99%, or 100%) identical to:
(SEQ ID NO: 5)
GGSGGSGGSGGSGGGSGGSGGSGGSG

125. A nanocage comprising at least one fusion protein of any one of claims 77 to 124 and at least one second nanocage monomer or subunit thereof that self-assembles with the fusion protein.

126. The nanocage of claim 125, wherein the fusion protein comprises a first nanocage monomer subunit, the second nanocage monomer or subunit thereof is a second nanocage monomer subunit, and the second nanocage monomer subunit self-assembles with the fusion protein to form the nanocage monomer.

127. The nanocage of claim 125 or 126, wherein from about 1% to about 100%, such as from about 1%, 4%, 8%, 10%, 12%, 15%, 20%, 25%, 30%, 35%, 40%, 45%, 50%, 55%, 60%, 65%, 70%, 75%, 80%, 85%, 90%, or 95%, to about 4%, 8%, 10%, 12%, 15%, 20%, 25%, 30%, 35%, 40%, 45%, 50%, 55%, 60%, 65%, 70%, 75%, 80%, 85%, 90%, 95%, or 100%, such as from about 20% to about 80%, of the nanocage monomers or subunits thereof is comprised within the fusion protein of any one of claims 77 to 124.

128. The nanocage of claim 125 or 126, wherein each nanocage monomer or subunit thereof is comprised within a fusion protein of any one of claims 77 to 124.

129. The nanocage of any one of claims 125 to 128, comprising 1 bioactive moiety or at least 2, 3, 4, 5, 6, 7, 8, 9, or 10 different bioactive moieties, such as 2 or 3 different bioactive moieties.

130. The nanocage of any one of claims 125 to 129, wherein the nanocage is multivalent.

131. The nanocage of any one of claims 125 to 130, wherein the nanocage is multispecific.

132. The nanocage of any one of claims 125 to 131, wherein at least one bioactive moiety decorates the exterior surface of the nanocage and at least one scFc fragment decorates the exterior surface of the nanocage.

133. The nanocage of claim 132, wherein at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 bioactive moieties decorate the exterior surface of the nanocage and at least two scFc fragments decorate the exterior surface of the nanocage.

134. The nanocage of any one of claims 125 to 133, comprising:
(a) at least one nanocage monomer fused to a first antigen-binding moiety, such as a Fab,
(b) at least one N-subunit fused to a second antigen-binding moiety, such as a Fab, and
(c) and at least one C-subunit fused to:
(i) a third antigen-binding moiety, such as a Fab at one terminus and
(ii) a scFc fragment at the other terminus.

135. The nanocage of claim 134, wherein each antigen-binding moiety is linked to the N-terminus of the nanocage monomer or subunit thereof and wherein the scFc fragment is linked to the C-terminus of the C-subunit.

136. The nanocage of claim 134 or 135, wherein the first and/or second antigen-binding moiety binds specifically to an antigen associated with an antibody-preventable and/or antibody-treatable condition.

137. The nanocage of claim 136, wherein the antigen is associated with an infectious agent, including a virus, bacteria, a parasite, a fungus, or a yeast, a cancer, or an immune disease, including an autoimmune disease.

138. The nanocage of claim 137, wherein each antigen-binding moiety is a different HIV-1-specific Fab.

139. The nanocage of claim 138, wherein the HIV-1-specific Fab binds to BG505 SOSIP_D368R, 93TH057 gp120, and/or an MPER peptide.

140. The nanocage of claim 139, wherein the HIV-1 specific Fab comprises PGDM1400 Fab, 10E8v4 Fab, and/or N49P7 Fab.

141. The nanocage of claim 140, comprising PGDM1400 Fab, 10E8v4 Fab, and N49P7 Fab.

142. The nanocage of any one of claims 125 to 141, comprising at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 antigen-binding moieties.

143. The nanocage of any one of claims 125 to 142, comprising at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 scFc fragments.

144. The nanocage of any one of claims 125 to 143, carrying a cargo molecule, such as a pharmaceutical agent, a diagnostic agent, and/or an imaging agent.

145. The nanocage of claim 144, wherein the cargo molecule is not fused to the fusion protein and is contained in the nanocage internally or wherein the cargo molecule is linked to the fusion protein or bound to the nanocage either internally or externally.

146. The nanocage of claim 144, wherein the cargo molecule is a protein and is fused to the fusion protein such that the cargo molecule is contained in the nanocage internally.

147. The nanocage of any one of claims 144 to 146, wherein the cargo molecule comprises a fluorescent protein, such as GFP, EGFP, Ametrine, and/or a flavin-based fluorescent protein, such as a LOV-protein, such as iLOV.

148. The nanocage of any one of claims 125 to 147, wherein the nanocage exhibits pan-virus neutralization breadth.

149. The nanocage of any one of claims 125 to 148, wherein the nanocage exhibits an average median IC₅₀ value against a multiclade panel of 118 pseudoviruses (PsV) of less than about 0.1 ug/mL, such as less than about 0.01 ug/mL, such as less than about 0.001 ug/mL.

150. The nanocage of any one of claims 125 to 149, wherein the nanocage exhibits an average median IC₅₀ value against a multiclade panel of 118 pseudoviruses (PsV) of less than about 42 pM, such as less than about 4.2 pM, such as less than about 0.42 pM.

151. The nanocage of any one of claims 125 to 150, wherein the nanocage exhibits an average median IC₅₀ value against a multiclade panel of 118 pseudoviruses (PsV) that is at least about 10, at least about 100, at least about 1000, at least about 10,000, or at least about 100,000 more potent than a cocktail of the corresponding bNAbs on a mass and/or molar basis.

152. A therapeutic or prophylactic composition comprising the nanocage of any one of claims 50 to 76 and 125 to 151.

153. The composition of claim 152, wherein the composition is for treating and/or preventing an antibody-preventable and/or antibody-treatable condition.

154. The composition of claim 153, wherein the antigen is associated with an infectious agent, including a virus, bacteria, a parasite, a fungus, or a yeast, a cancer, or an immune disease, including an autoimmune disease.

155. The composition of claim 154, wherein the composition is for treating and/or preventing an HIV-1-related condition.

156. A nucleic acid molecule encoding the fusion protein of any one of claims 1 to 49 and 77 to 124.

157. A vector comprising the nucleic acid molecule of claim 156.

158. A host cell comprising the vector of claim 157 and producing the fusion protein of any one of claims 1 to 49 and 77 to 124.

159. A method for treating and/or preventing a condition, the method comprising administering the nanocage of any one of claims 50 to 76 and 125 to 151 or the composition of any one of claims 152 to 155 to a subject in need thereof.

160. The method of claim 159, wherein the condition is an HIV-1-related condition.

161. Use of the nanocage of any one of claims 50 to 76 and 125 to 151 or the composition of any one of claims 152 to 155 for treating and/or preventing a condition.

162. The use of claim 161, wherein the condition is an HIV-1-related condition.

163. The nanocage of any one of claims 50 to 76 and 125 to 151 or the composition of any one of claims 152 to 155 for use in treating and/or preventing a condition.

164. The nanocage of claim 163, wherein the condition is an HIV-1-related condition.

165. The nanocage of any one of claims 50-76, 125-151, or 163-164, wherein the nanocage does not include any ferritin heavy chains.

166. The nanocage of any one of claims 50-76, 125-151, or 163-164, wherein the nanocage does not include any components capable of ferroxidase activity.

167. The nanocage of any one of claims 50-76, or 163-166, wherein the nanocage comprises at least one bioactive moiety comprising an antibody or antigen-binding fragment thereof, and the ratio of the total number of bioactive moieties to the number of Fc dimers is 6:1.

168. The nanocage of any one of claims 125-151, or 163-166, wherein the nanocage comprises at least one bioactive moiety comprising an antibody or antigen-binding fragment thereof, and the ratio of the total number of bioactive moieties to the number of scFcs is 3:1.

169. The nanocage of any one of claims 50-76, or 163-166, wherein the nanocage comprises at least one bioactive moiety comprising an antibody or antigen-binding fragment thereof, and the ratio of the total number of bioactive moieties to the number of Fc dimers or scFcs is at least 7:1.

170. The nanocage of any one of claims 125-151, or 163-166, wherein the nanocage comprises at least one bioactive moiety comprising an antibody or antigen-binding fragment thereof, and the ratio of the total number of bioactive moieties to the number of Fc dimers or scFcs is at least 4:1.
